SEQUENCE LISTING (104 sequences)

SEQ ID NO: 1 - DNA, 2163 bp, Cannabis sativa
atgggaaaaaattataagtcacttgacagtgtggttgctagtgatttcatagccttgggtatcacatctgaggtagcggagactcttcatgggcgtttagctgagattgtgtgtaattacggggcggccacccctcagacttggatcaatatagcaaaccacatcttatctcctgatctacccttttctctacaccaaatgctgttctatgggtgttacaaagatttcgggcccgcaccgcccgcatggataccggatccagagaaagtcaaatccaccaacttaggcgcgttgcttgaaaaaagaggcaaggaattcctgggcgtcaagtacaaggaccccatctcatctttctcccacttccaagagttctctgtacgtaatccggaggtttactggcgtaccgtccttatggatgagatgaagatatcattctccaaggacccggaatgtatactaagaagggatgacattaacaacccagggggcagtgagtggcttccgggtggataccttaatagtgcaaaaaactgtcttaacgtgaactcaaacaaaaagttaaacgatacaatgatcgtgtggcgtgatgagggcaatgatgaccttccgctaaataagttgacgcttgatcagctaagaaagcgtgtctggcttgtcggatatgccctagaggagatgggccttgagaagggatgcgcgatcgccatagatatgcctatgcatgtcgatgctgtggttatatatctggccatcgttctagcgggctatgttgttgtgagtattgccgatagcttcagtgcacccgaaatatcaacgagactgcgtttgtccaaagccaaagcaatattcacacaggatcatatcattagaggaaagaagcgtattccgctatattcaagggtggtggaggccaaaagcccgatggctatagtgataccctgttcaggcagcaacatcggagctgaattaagagatggagatatatcctgggactattttttagagagggcgaaagaatttaagaactgcgagttcaccgcaagggaacagccagtggatgcgtatacaaacatactattcagttcaggtactacgggagaaccaaaggctataccctggacgcaggcaaccccgctaaaagctgccgctgacgggtggtctcatttggatatcaggaagggtgatgttatcgtatggccaacaaatcttggatggatgatgggaccgtggcttgtgtacgctagcttgttaaatggagcttcaatcgctctatataatggttcccctcttgtcagcggttttgcgaagttcgtacaggatgccaaggtaactatgctgggagtcgttcctagcatcgtcaggagttggaaatcaactaactgtgtgtctggttacgactggtccactatcagatgttttagctccagtggtgaggcctccaacgtagatgagtacctttggcttatgggacgtgccaactataaaccggtaatagagatgtgtgggggtactgaaataggaggagcgttttcagctggcagttttcttcaggctcaaagcttgtctagtttctcttcacagtgtatgggctgtacgttatacattcttgataagaacggttatccaatgcccaaaaacaaaccaggtataggagaattggcgctgggtccagtcatgtttggcgctagtaaaactctactgaacggaaatcatcacgacgtttactttaaaggtatgcctactcttaatggagaagtacttaggcgtcatggcgatatctttgagttgacatccaatggctactatcacgcccatggcagggcggatgacaccatgaacatcggggggatcaaaatctccagtatagagatcgagagagtgtgtaacgaggtagacgaccgtgtattcgagacaacagccattggggttccacccctaggtggtggccccgaacaacttgttatcttttttgtgctgaaggactcaaatgacactaccattgatttaaatcaactacgtttgtcattcaatctgggattacaaaagaagttgaatcctttattcaaggtcacaagagtagtaccccttagttccctgccaagaactgcgacaaacaagataatgcgtagagtgctaaggcagcagtttagtcattttgagtaa

SEQ ID NO: 2 - protein, 720 aa, Cannabis sativa
MGKNYKSLDSVVASDFIALGITSEVAETLHGRLAEIVCNYGAATPQTWINIANHILSPDLPFSLHQMLFYGCYKDFGPAPPAWIPDPEKVKSTNLGALLEKRGKEFLGVKYKDPISSFSHFQEFSVRNPEVYWRTVLMDEMKISFSKDPECILRRDDINNPGGSEWLPGGYLNSAKNCLNVNSNKKLNDTMIVWRDEGNDDLPLNKLTLDQLRKRVWLVGYALEEMGLEKGCAIAIDMPMHVDAVVIYLAIVLAGYVVVSIADSFSAPEISTRLRLSKAKAIFTQDHIIRGKKRIPLYSRVVEAKSPMAIVIPCSGSNIGAELRDGDISWDYFLERAKEFKNCEFTAREQPVDAYTNILFSSGTTGEPKAIPWTQATPLKAAADGWSHLDIRKGDVIVWPTNLGWMMGPWLVYASLLNGASIALYNGSPLVSGFAKFVQDAKVTMLGVVPSIVRSWKSTNCVSGYDWSTIRCFSSSGEASNVDEYLWLMGRANYKPVIEMCGGTEIGGAFSAGSFLQAQSLSSFSSQCMGCTLYILDKNGYPMPKNKPGIGELALGPVMFGASKTLLNGNHHDVYFKGMPTLNGEVLRRHGDIFELTSNGYYHAHGRADDTMNIGGIKISSIEIERVCNEVDDRVFETTAIGVPPLGGGPEQLVIFFVLKDSNDTTIDLNQLRLSFNLGLQKKLNPLFKVTRVVPLSSLPRTATNKIMRRVLRQQFSHFE

SEQ ID NO: 3 - DNA, 1158 bp, Cannabis sativa
atgaaccacttgagagcagaagggccggccagtgtactggctatagggacagccaaccccgaaaatatactgttgcaagatgagttcccagattattactttagagtgactaaatccgagcacatgacgcaacttaaggagaaattcaggaaaatatgcgacaaatctatgattagaaaaagaaattgtttcttaaatgaggagcatctaaagcagaatccccgtctggtagaacatgaaatgcaaactttggacgcgcgtcaagacatgctagttgtcgaagtgcctaaattaggcaaagacgcgtgtgcaaaggctataaaagagtggggccaaccgaagtccaaaattacacacctaatattcacttctgcgtccaccaccgacatgcccggagccgactaccactgtgcgaaacttctaggcctatccccttcagtcaagcgtgtaatgatgtatcaactggggtgctacggaggaggcaccgttttgaggattgcaaaggatatcgctgaaaataacaagggggctcgtgtacttgctgtgtgctgtgatatcatggcctgccttttcagaggcccctcagagtcagatcttgaactgttagtaggtcaggctatcttcggagatggcgctgcagccgtcatagttggggcggagcctgacgaatcagttggggagaggcccattttcgagctggtcagtacgggacagaccatcttgccaaatagcgagggcacgatcggaggccacataagggaggcgggtttgatatttgaccttcataaggatgtaccgatgttgatctccaataatattgagaagtgtcttattgaagcatttacccctattggtatttcagactggaacagtatcttctggattacgcatccgggaggtaaggcgattcttgataaagtcgaagaaaagctacacctgaagtcagacaagttcgttgactccagacacgttctttcagagcacggcaacatgagttcttccaccgtccttttcgtaatggacgagctgaggaaacgtagccttgaggaaggtaaaagtacgacaggagatgggtttgagtggggagtgttgtttggcttcggcccagggttaacagttgaacgtgtagtcgttagatctgtccctattaaatactaa

SEQ ID NO: 4 - protein, 385 aa, Cannabis sativa
MNHLRAEGPASVLAIGTANPENILLQDEFPDYYFRVTKSEHMTQLKEKFRKICDKSMIRKRNCFLNEEHLKQNPRLVEHEMQTLDARQDMLVVEVPKLGKDACAKAIKEWGQPKSKITHLIFTSASTTDMPGADYHCAKLLGLSPSVKRVMMYQLGCYGGGTVLRIAKDIAENNKGARVLAVCCDIMACLFRGPSESDLELLVGQAIFGDGAAAVIVGAEPDESVGERPIFELVSTGQTILPNSEGTIGGHIREAGLIFDLHKDVPMLISNNIEKCLIEAFTPIGISDWNSIFWITHPGGKAILDKVEEKLHLKSDKFVDSRHVLSEHGNMSSSTVLFVMDELRKRSLEEGKSTTGDGFEWGVLFGFGPGLTVERVVVRSVPIKY

SEQ ID NO: 5 - DNA, 306 bp, Cannabis sativa
atggctgtcaagcaccttatcgtactgaaattcaaggacgaaattacagaagcccagaaagaagaatttttcaagacctatgtaaacctggtgaatatcatccctgcgatgaaagatgtttattggggcaaagacgtcacccaaaaaaacaaagaagaaggttacactcacatcgtcgaagtcactttcgagtcagtagagacgatccaagattacataatacacccggcccatgtgggattcggagacgtttaccgtagcttctgggaaaaacttttgattttcgactataccccgagaaaataa

SEQ ID NO: 6 - protein, 101 aa, Cannabis sativa
MAVKHLIVLKFKDEITEAQKEEFFKTYVNLVNIIPAMKDVYWGKDVTQKNKEEGYTHIVEVTFESVETIQDYIIHPAHVGFGDVYRSFWEKLLIFDYTPRK

SEQ ID NO: 7 - DNA, 1517 bp, Cannabis sativa
atcaataataatcttcatgggactctcattagtttgtaccttttcatttcaaactaattatcatactttattaaaccctcataataagaatcccaaaaactcattattatcttatcaacaccccaaaacaccaataattaaatcctcttatgataattttccctctaaatattgcttaaccaagaactttcatttacttggactcaattcacacaacagaataagctcacaatcaaggtccattagggcaggtagcgatcaaattgaaggttctcctcatcatgaatctgataattcaatagcaactaaaattttaaattttggacatacttgttggaaacttcaaagaccatatgtagtaaaagggatgatttcaatcgcttgtggtttgtttgggagagagttgttcaataacagacatttattcagttggggtttgatgtggaaggcattctttgctttggtgcctatattgtccttcaatttctttgcagcaatcatgaatcaaatttacgatgtggacatcgacaggataaacaagcctgatctaccactagtttcaggggaaatgtcaattgaaacagcttggattttgagcataattgtggcactaactgggttgatagtaactataaaattgaaatctgcaccactttttgttttcatttacatttttggtatatttgctgggtttgcctattctgttccaccaattagatggaagcaatatccttttaccaattttctaattaccatatcgagtcatgtgggcttagctttcacatcatattctgcaaccacatcagctcttggtttaccatttgtgtggaggcctgcttttagtttcatcatagcattcatgacagttatgggtatgactattgcttttgccaaagatatttcagatattgaaggcgacgccaaatatggggtatcaactgttgcaaccaaattaggtgctaggaacatgacatttgttgtttctggagttcttcttctaaactacttggtttctatatctattgggataatttggcctcaggttttcaagagtaacataatgatactttctcatgcaatcttagcattttgcttaatcttccagactcgtgagcttgctctagcaaattacgcctcggcgccaagcagacaattcttcgagtttatctggttgctatattatgctgaatactttgtatatgtatttatataagaccataatataacatatatatgtttattacataaaattgggacacaaaaacgtcaattatttggacaaaagtactcagaaagacctctttcactacaaggggaggccatttagttatacttgggtttcaatcaacaaatttataaatttttaagattttatttacaaaacattttcatgtgtaattaaatcgatcgtcatttattttttggatacaacttggttcaacttattttaattagagtgcttcgtaatttaactacaattatagaagggcattttataaaaatactggatttggggt

SEQ ID NO: 8 - protein, 398 aa, Cannabis sativa
MGLSLVCTFSFQTNYHTLLNPHNKNPKNSLLSYQHPKTPIIKSSYDNFPSKYCLTKNFHLLGLNSHNRISSQSRSIRAGSDQIEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 9 - DNA, 951 bp, artificial
  Synthetic polynucleotide
atcgaaggttcacctcatcatgaaagtgataacagcatagccacgaagattttgaatttcggccatacttgttggaagctacagaggccgtacgtcgttaaggggatgatttccattgcgtgcggtctgtttggcagggaattatttaacaacagacacttattcagttggggcctgatgtggaaggccttcttcgctcttgtacccattctgtccttcaacttttttgcagcgatcatgaatcaaatatacgatgtagacatcgatagaataaacaagcccgatttacctctggtatcaggcgaaatgagcatcgaaactgcgtggattttatcaatcatcgttgcattgactgggctgatagtgaccataaagttaaagtcagccccgttgtttgtcttcatatacatcttcggcattttcgcgggctttgcgtatagtgtacctcccattagatggaagcagtacccgtttactaactttcttattacaattagcagccatgtcggtcttgcattcacgtcctactcagccaccacatccgcactggggctaccgtttgtgtggcgtccagccttcagcttcatcatcgcattcatgacagtaatgggtatgacgatagcttttgcaaaggatataagtgatatcgagggtgacgctaagtatggagtgtctactgtggccacgaagctgggggcccgtaatatgactttcgtggtatcaggtgtactattgcttaattaccttgtttctatatcaatcggaattatttggccacaagttttcaaatccaatataatgatcctatcacacgctattttagcgttttgtttgatatttcagactagagagcttgcactagcgaattacgcgagtgccccgagtaggcagtttttcgagttcatatggctattatactatgctgagtactttgtttacgtatttatttaa

SEQ ID NO: 10 - protein, 316 aa, artificial
  Synthetic polypeptide
IEGSPHHESDNSIATKILNFGHTCWKLQRPYVVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 11 - DNA, 1635 bp, Cannabis sativa
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcttccacggcatcgtcattaa

SEQ ID NO: 12 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 13 - DNA, 1551 bp, Artificial
  Synthetic polynucleotide
aacccccgtgaaaattttttgaaatgtttctctcaatacatacccaacaatgcaaccaacttaaagctggtatatactcaaaacaaccccctatatatgtctgttctaaatagtactatccataacttacgtttcacctcagataccacccctaaaccgctggtcatcgtgactccgtctcatgtttcacacatacagggcacgatattgtgctcaaaaaaggtcgggttacagattcgtacccgttcaggaggtcatgatagtgagggaatgtcttacatctcccaggtcccttttgtaattgtcgaccttcgtaatatgagatccataaagatcgacgttcattcacagacggcgtgggtagaggctggtgcaaccctaggtgaagtctactactgggtcaacgaaaaaaacgagaacttatcattagctgcggggtattgccctacagtttgtgccggaggtcattttggaggtggaggctacgggccactgatgaggaactacggtctggcagcagacaacattatagatgcacacctagtgaacgtgcatggtaaagttttagatagaaagtccatgggagaagatttgttttgggcactacgtggaggaggggctgagtcattcgggattattgtagcgtggaagatccgtctggtcgcagtccctaaatctacgatgttttccgtgaaaaagattatggaaattcacgagctagtgaaacttgtcaataagtggcagaatatagcatacaaatatgacaaggatctattgttgatgacgcatttcatcacaagaaacattacggacaatcaaggtaagaataagacggctattcacacttacttcagctccgtttttctaggaggggtagattccctagttgacctgatgaataagagttttcccgagttgggtattaaaaaaactgattgtagacagctgtcttggatcgacacaatcatattctactctggtgtggtaaactatgacaccgataatttcaacaaagaaatcttactggatagatcagccggtcaaaacggcgcgtttaaaatcaagctggattacgtaaagaagcctatacccgaatccgtatttgtacagattctggaaaagttatacgaggaagacattggggcgggtatgtacgctctttacccttacggcgggatcatggatgagatttccgaaagtgctatcccgttccctcatcgtgctggcattctgtacgagttatggtatatttgcagttgggagaagcaggaggataatgaaaagcacctaaattggattcgtaatatttataatttcatgactccctatgttagtaagaaccccagactggcctaccttaattatagagacctggacatcgggataaatgatccgaagaacccaaataactatacgcaggccaggatttggggggaaaagtatttcggaaagaactttgacagactggtgaaagttaagaccctggtggatccaaataattttttcaggaacgagcagagtattcccccgcttccacgtcacaggcattaa

SEQ ID NO: 14 - protein, 516 aa, Artificial
  Synthetic polypeptide
NPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 15 - DNA, 1653 bp, Taxus x media
atggaggacttgaagaggtgcccagctaactaccctccattgacccctatcggtttcatcgaaagggctgctaccgtttacggagactgcaccagtttggtctacaataccactagattcacttggagtcagaccttcaatagatgcttgaaaatggccagtgccttgagttctagaaacatcagtagaggagacgtcgttagtgttgtcgccccaaatgtcccagccatctacgagatgcatttcgccgtcccaatggctggtgccgtccttaacaacgtcaatatcagacttgatgctagaaccatggccgctcaattcacccactgtgaaccaaaattccttttcgtcgactaccagttcttgcctcttgttagagaagccttgagtgaaatcggtcacaagccatgtgtcgtcgtcatcgaggaacttgacaatggtagggagatggccatgagtggtgccttgacttacgagggattgatcggtgagggagacccagaattcgagattagatggccagaagacgaatggcaagccgccgttcttaactacaccagtggtaccaccagtgcccctaagggtgtcgtccattctcacagaggactttacaccatggctatggacaacttgcttatgtggggaatgaccactagaccagtttacctttggaccttggccatgttccacgctaatggttggtgcttcccatggactttggctgctgtcggtggtaccaacatttgccttaggaagtttgatgccaagaccatctttgatgccttggccgaacacggtgtcactcatatgtgcggtgctccagtcgtcttgtctatgatggccaacgcccacccatctgaacacaagccagttgctggaagggtcgagatccttactgctggtgccccaccaccagctgctatcctttggaaggtcgaggacatgggttttgccgtcacccatggatacggtcttaccgaaaccgctggtttggtcgtcagttgcgcttggaaagccgagtgggatactttgccagccgaggagagagctagattgaaagctagacaaggtgccagaaacgtctctcttgccgaggtcgacgtcaaggatccaacttctatggccagtgtcgctaaggacggtcttcagatgggagaggtcatgcttaggggaagttctgtcatgaaaggttaccttaagaacgagcagatgactgcccttgctatggagggaggttggttcagaactggtgacgtcggtgtcttgcacccagacggataccttgaaatcaaggatagatctaaggacgtcatcatcagtggtggtgagaacattagtagtgtcgaggtcgagtctgtcctttacagtcaccctcttatcgttgaggccgctgtcgtcggtagaccagatagtttctggggtgagaccccttgcgctttcgtcaatatcggaaacaaaggtaccgaagtcttgaccgaggcccaagttatctctttctgtagagagaggatcgcccacttcatggctcctaaatctgttatttttgttaaagaattgccaaagacttctaccggaaaagtccaaaaataccttcttagggatattgccaagaagcctccaagttgtagacttcaagttcccggttactcttaa

SEQ ID NO: 16 - protein, 550 aa, Taxus x media
MEDLKRCPANYPPLTPIGFIERAATVYGDCTSLVYNTTRFTWSQTFNRCLKMASALSSRNISRGDVVSVVAPNVPAIYEMHFAVPMAGAVLNNVNIRLDARTMAAQFTHCEPKFLFVDYQFLPLVREALSEIGHKPCVVVIEELDNGREMAMSGALTYEGLIGEGDPEFEIRWPEDEWQAAVLNYTSGTTSAPKGVVHSHRGLYTMAMDNLLMWGMTTRPVYLWTLAMFHANGWCFPWTLAAVGGTNICLRKFDAKTIFDALAEHGVTHMCGAPVVLSMMANAHPSEHKPVAGRVEILTAGAPPPAAILWKVEDMGFAVTHGYGLTETAGLVVSCAWKAEWDTLPAEERARLKARQGARNVSLAEVDVKDPTSMASVAKDGLQMGEVMLRGSSVMKGYLKNEQMTALAMEGGWFRTGDVGVLHPDGYLEIKDRSKDVIISGGENISSVEVESVLYSHPLIVEAAVVGRPDSFWGETPCAFVNIGNKGTEVLTEAQVISFCRERIAHFMAPKSVIFVKELPKTSTGKVQKYLLRDIAKKPPSCRLQVPGYS

SEQ ID NO: 17 - DNA, 1722 bp, Humulus lupulus
atggataactatagaaggctccacactccggttgctctctgcgttgccagtccacctgcgccacccaccacatcatggaagtcaatggagggcttagttcagtgctctgcaaatcatgttcctctctctcccattaccttcttggagcgttcttccaaggcttacagagacaacacctctcttgtctatggctctgtcagatacacttgggcccaaactcaccatcgctgtctcaagctagcttctgctctcacaacccacttgggaatttcaccaggggatgtggtggctaccttctcttacaacctaccagaaatctacgagcttcattttgcagtcccaatggctggtgggattctctgtacactcaacgctcgcaacgactcggccatggtgtcgacgctgctagcacactcggaagccaaactcatctttgtggaaccccagttactggaaacggctcgggcagctcttgatcttctcgcccaaaaggacataaagcctccaactttggtcttactaaccgattcggaaagcttcacttcaagctcatacgatcactataatcatctgttggccaatgggtctgatgacttcgaaataagacggcctaagaacgaatgggatcccatcagcataaactacacctcaggcaccactgcacgccccaaagctgtcgtttacagccaccgtggggcatatctgaactccatagccacagttttgcttcacgggatggggacaacgtctgtttatctttggtcagtgcccatgtttcattgcaacggctggtgttttccatggggggctgcagctcagggcgccaccaacatatgcataagaaaagtctctcccaaagccatttttgacaacatacatttgcataaggttacacactttggagctgcaccaactgtcttgaacatgattgtgaactcgccggaaggcaaccttcacaccccgcttccccacaaggtggaggtcatgacaggaggttcaccgccaccgcccaaggtcattgcgaggatggaagagatggggtttcaagtgaatcacatttatggcctcacggaaacttgtggtcctgctgctaattgtgtatgcaaacctgaatgggatgcactgcagccagaggaacggtatgccttgaaagctcgtcaaggattaaaccatctggcgatggaggagatggacgtgagagacccggtgaccatggaaagtgttagggccgatggtgcaacgattggtgaggttatgttcagaggaaacactgtgatgagtggctactttaaagacttgaaggcgaccgaggaggctttcgagggaggttggtttcgtagtggggatcttggtgtgaaacatgaggatggttatattcaacttaaggatcggaagaaggatgtggtgatatcaggaggggagaatatcagtacagttgaagttgagactgtgttgtatagccacgaagcagtgctcgaggctgctgtggtggcgcgccctgataagctttggggggagacgccttgtgcttttgtgacacttaaggagggatttgataatgatgtaagtgctgaccaaattatcaaattctgtagagatcgtttgccccattacatggctcccaagacagtagtgtttgaagagttaccaaagacttcaacaggaaagatacagaagtatattctgaaagaaaaagcaatggccatgggcagcctttcttga

SEQ ID NO: 18 - protein, 573 aa, Humulus lupulus
MDNYRRLHTPVALCVASPPAPPTTSWKSMEGLVQCSANHVPLSPITFLERSSKAYRDNTSLVYGSVRYTWAQTHHRCLKLASALTTHLGISPGDVVATFSYNLPEIYELHFAVPMAGGILCTLNARNDSAMVSTLLAHSEAKLIFVEPQLLETARAALDLLAQKDIKPPTLVLLTDSESFTSSSYDHYNHLLANGSDDFEIRRPKNEWDPISINYTSGTTARPKAVVYSHRGAYLNSIATVLLHGMGTTSVYLWSVPMFHCNGWCFPWGAAAQGATNICIRKVSPKAIFDNIHLHKVTHFGAAPTVLNMIVNSPEGNLHTPLPHKVEVMTGGSPPPPKVIARMEEMGFQVNHIYGLTETCGPAANCVCKPEWDALQPEERYALKARQGLNHLAMEEMDVRDPVTMESVRADGATIGEVMFRGNTVMSGYFKDLKATEEAFEGGWFRSGDLGVKHEDGYIQLKDRKKDVVISGGENISTVEVETVLYSHEAVLEAAVVARPDKLWGETPCAFVTLKEGFDNDVSADQIIKFCRDRLPHYMAPKTVVFEELPKTSTGKIQKYILKEKAMAMGSLS

SEQ ID NO: 19 - DNA, 1632 bp, Callitris macleayana
atggaggaattgaaaaggtgcccagctaactaccctccattcacccctatcggtttcgtcgagagagccgccactgtctacagtgattgcaccagtgttgtctacagtaccactagattcacttggagtcaaaccttccagaggtgtaggaagcttgcctctgctttggccttgaggaacgtctgtagaggtgacgtcgtcagtgttgttgctccaaacgtcccagctatgtacgagatgcactttggtgtccctatgagtggtgccgtccttaacaacttgaacactagattggatcctaggaccatggctgcccaaatcagtcactgccagcctaaaatcatcttcgctgattatcaattccttagtttggttaatgagaccttgtctttgttgcagcacaagccacctttggtcgttatcgaggagttgcagaacggtagggaaattggaaggggtgccgagttgacctacgaaggtttgcttagagagggtgacccagagttcgagattagatggccagaggatgagtggcaagccgctgtcttgaactacacctctggtaccacctctgctcctaagggagtcgttcagtctcacagaggaatctacgccatggctcttgacaatttgactatgtggcagatgggaaggagacctatctacctttggaccttggccatgttccacgccaatggttggagtcttccttggactttggctgccgttggaggaaccaatatctgccttaggaagttcgacgccaccaccatctttgactctatcgccgagcacaacgtcacccacatgtgtggagccccagttgtcttgtctatgatggccaacgctgatccagctgacagaaagcaccttgcccacaaggttgagatcttgactgccggtgctccaccaccagctgctgtcttgtggaagatggaggagatgggattctctatcacccacggatacggacttaccgaaaccgccggtcttgttgtctcttgcgcttggaaaaccgagtgggatggtcttcccggtaaggagaaggctaggttgaagagtaggcaaggtgttagaaatctttctcttgccgaggtcgacgttaaaaacccagtcaccatggccagtgtcaagagagacggtgttgagatgggtgaggtcatgatgaggggagccagtgttatgaagggttacttgaagaacgacgacatgaccgctagagctatggagggtggttggtttagaaccggtgacgtcgctgtcattcacccagatggatatatcgaaattaaagataggagtaaggatgttattatttctggtggtgagaatatttcttctgttgaggttgaaagtgttctttactctcacccatctgtcgtcgaagctgctgtcgtcgctaggccagatagtttttggggagagaccccttgcgcctttgtcagtgtcaagaacaacggtggagagggtaacaaagaagttttgtctgagggtgaaatcatcgagttctgtaggaaacacttggcccacttcatggctcctaagagtgttatttttatgcaagaacttccaaagacttctactggaaagattcagaaattcgttttgagagaaatggctaaaaagctttaa

SEQ ID NO: 20 - protein, 543 aa, Callitris macleayana
MEELKRCPANYPPFTPIGFVERAATVYSDCTSVVYSTTRFTWSQTFQRCRKLASALALRNVCRGDVVSVVAPNVPAMYEMHFGVPMSGAVLNNLNTRLDPRTMAAQISHCQPKIIFADYQFLSLVNETLSLLQHKPPLVVIEELQNGREIGRGAELTYEGLLREGDPEFEIRWPEDEWQAAVLNYTSGTTSAPKGVVQSHRGIYAMALDNLTMWQMGRRPIYLWTLAMFHANGWSLPWTLAAVGGTNICLRKFDATTIFDSIAEHNVTHMCGAPVVLSMMANADPADRKHLAHKVEILTAGAPPPAAVLWKMEEMGFSITHGYGLTETAGLVVSCAWKTEWDGLPGKEKARLKSRQGVRNLSLAEVDVKNPVTMASVKRDGVEMGEVMMRGASVMKGYLKNDDMTARAMEGGWFRTGDVAVIHPDGYIEIKDRSKDVIISGGENISSVEVESVLYSHPSVVEAAVVARPDSFWGETPCAFVSVKNNGGEGNKEVLSEGEIIEFCRKHLAHFMAPKSVIFMQELPKTSTGKIQKFVLREMAKKL

SEQ ID NO: 21 - DNA, 1632 bp, Diselma archeri
atggaggagcttaaaaggtgcccagccaactacccaccattcaccccaatcggtttcgttgaaagggctgccaccgtctattctgattgtacctctgtcgtttacaacaccactagattcacttggtctcagaccttccagaggtgcagaaagttggcctctgcccttgccttgaggaacatctgtaggggagacgttgttagtgtcgtcgccccaaatgtcccagctatgtacgagatgcactttgccgtcccaatgtctggtgctgtcttgaacaacttgaacactagattggatgctagaactatggctgctcagatctctcattgcgaaccaaagattatcttcgctgattaccagtttttgtctttggttattgaaaccctttctttgttgaaacataagccaccattggtcgtcatcgaggaaatgcagaacggtagggaaatcggtagaggagccgagttgacctacgagggtttgcttagggagggtgatccagaattcgagattaggtggccagaagacgaatggcaagccgctgtccttaactacaccagtggaaccaccagtgcccctaagggagttgtccaaagtcatagaggtatctacgctatggccttggacaacttgaccatgtggcagatgggaaggaggcctatctacttgtggaccttggccatgttccacgccaatggttggagtcttccttggactttggctgccgttggtgccaccaacatttgcttgaggaaatttgatgctcaaactatctttgattctatcgccgaacacaatgttacccatatgtgcggagccccagttgtcttgagtatgatggccaacgccgacccagctgatagaaagcacttggcccagagggtcgaaattttgactgccggtgctccaccaccagctgctgtcttgtggaagatggaggagatgggattctctatcacccacggttacggtttgaccgaaaccgccggtttggttgtctcttgcgcttggaaaaccgagtgggatggtttgcccggtaaagagaaggctagattgaagagtaggcaaggtgttaggaacttgagtcttgccgaggtcgacgtcaagaatccagttaccatggcttctgtcaagaggtacggtgtccaaatgggagaggtcatgatgaggggtgccagtatcatgaagggttacttgaaaaacgacgacatgaccgctagagtcatggagggtggttggttcagaaccggagacgtcgctgttgtccacccagatggttacattgaaattaaagatagatctaaggacgtcatcatctctggaggagagaacattagttctgttgaggttgagtctgtcttgtacagtcacccaagtgtcgttgaagctgccgtcgtcgctagaccagatagtttctggggagagactccttgcgccttcgtctctgtcaagaaccacggtggtgagggtaataaagaagttctttctgagagggaaatcatcgaattctgtagaaagcaccttgcccacttcatggccccaaaaagtgtcattttcatgcaagaattgcctaagacctctactggtaagattcaaaaatttgttttgagagaaatggctaaaaagctttaa

SEQ ID NO: 22 - protein, 543 aa, Diselma archeri
MEELKRCPANYPPFTPIGFVERAATVYSDCTSVVYNTTRFTWSQTFQRCRKLASALALRNICRGDVVSVVAPNVPAMYEMHFAVPMSGAVLNNLNTRLDARTMAAQISHCEPKIIFADYQFLSLVIETLSLLKHKPPLVVIEEMQNGREIGRGAELTYEGLLREGDPEFEIRWPEDEWQAAVLNYTSGTTSAPKGVVQSHRGIYAMALDNLTMWQMGRRPIYLWTLAMFHANGWSLPWTLAAVGATNICLRKFDAQTIFDSIAEHNVTHMCGAPVVLSMMANADPADRKHLAQRVEILTAGAPPPAAVLWKMEEMGFSITHGYGLTETAGLVVSCAWKTEWDGLPGKEKARLKSRQGVRNLSLAEVDVKNPVTMASVKRYGVQMGEVMMRGASIMKGYLKNDDMTARVMEGGWFRTGDVAVVHPDGYIEIKDRSKDVIISGGENISSVEVESVLYSHPSVVEAAVVARPDSFWGETPCAFVSVKNHGGEGNKEVLSEREIIEFCRKHLAHFMAPKSVIFMQELPKTSTGKIQKFVLREMAKKL

SEQ ID NO: 23 - DNA, 1707 bp, Humulus lupulus
atgggtatggttgggagagatatagacgatcttccgaagaacgccgccaattacacggcgttgacgccgctctggtttcttgagagagcggcgacggtacatccgacgagaacgtcggtgattcatggttctcgacactacacgtggcttcagacgtaccatcggtgtcgtcagttcgcctctgctctcaacaatcattccatcggcctcggcagcacggtagctgtaattgctccaaatgttcctgccctttatgaagctcattttgctgtaccaatggcaggggcagtggtgaattgtgtgaacattcgtctaaatgcatcaacaattgctttccttctgggtcattcatcagctgctgctgtgatggtagatcaggagtttttttccttggctgaggaagctttgaaaatcttagcacaggaaagcaaaagccattacaagcccccacttctagtggtaataggtgatgaaagttgtgatcctaagactcttgaatatgctttgaagacaggagccattgaatatgagaaatttctggaagggggtgaccctgaatttgattggaaaccaccagaggatgagtggcaaagcatttctttgggttacacttctggtacgacagcaagccccaagggggtggtgttgagccaccgtggagcgtatctgatgtctttgagtgcttctgttgtctgggggataaatgagggagctatatacttgtggactctacccatgttccattgcaacggttggtgttacacttggggtatggctgctttttgtggtacaaacatatgtttacgacaggttacagcaaagggtgtctattctgccatagccaagtatggtgttactcacttttgtgctgctcctgtggtactcaacaccatagtcaatgccccaccagaggaagctatcattcctctccctcatcttgtacatgtcatgactgctggtgctgctccacctccttcagttctctttgcaatgtccgaaaaaggcttcaaggtcgcccacacttatggtctctctgaaacttatggtccttccacgatatgtgcatggaagcctgaatgggattcacttcctcccatcaaacaagctcgattgaatgcacgccaaggtgttcgatacattgcattggagggcctcgatgttgtcgataccaaaacaatgaagcctgtccctgctgatggaactactatgggagagattgtcatgaggggaaatgctgtgatgaagggttacttaaagaatccaaaagctaacgaagaatctttcgctgatggttggtttcattcaggtgatctagcagtaaaacatccagatgggtacatagaaatcaaagacagatcaaaggacatcatcatatccggaggtgagaacataagtagcttggaggttgagaacactctgtatttgcacccagcagtattagaagtatctgttgtggccaggcccgatgagcgctggggcgagtctccatgtgctttcgtgacattgaagcccaatatagacaagtccaacgaacaagttttggctgaagatatcattaagttttgcaagtccaaaatgcctgcttattgggtccccaaatcagttgtatttggaccattgccaaagacagccactggtaagatacaaaagcatgtactaagggccaaggccaaagagatgggggcccttaagaagagcaacttataa

SEQ ID NO: 24 - protein, 568 aa, Humulus lupulus
MGMVGRDIDDLPKNAANYTALTPLWFLERAATVHPTRTSVIHGSRHYTWLQTYHRCRQFASALNNHSIGLGSTVAVIAPNVPALYEAHFAVPMAGAVVNCVNIRLNASTIAFLLGHSSAAAVMVDQEFFSLAEEALKILAQESKSHYKPPLLVVIGDESCDPKTLEYALKTGAIEYEKFLEGGDPEFDWKPPEDEWQSISLGYTSGTTASPKGVVLSHRGAYLMSLSASVVWGINEGAIYLWTLPMFHCNGWCYTWGMAAFCGTNICLRQVTAKGVYSAIAKYGVTHFCAAPVVLNTIVNAPPEEAIIPLPHLVHVMTAGAAPPPSVLFAMSEKGFKVAHTYGLSETYGPSTICAWKPEWDSLPPIKQARLNARQGVRYIALEGLDVVDTKTMKPVPADGTTMGEIVMRGNAVMKGYLKNPKANEESFADGWFHSGDLAVKHPDGYIEIKDRSKDIIISGGENISSLEVENTLYLHPAVLEVSVVARPDERWGESPCAFVTLKPNIDKSNEQVLAEDIIKFCKSKMPAYWVPKSVVFGPLPKTATGKIQKHVLRAKAKEMGALKKSNL

SEQ ID NO: 25 - DNA, 1620 bp, Amentotaxus argotaenia
atggaggaattgaagagatgcccagccaactaccctccattgacccctatcggattcatcgagagggccgctactgtctacggtgactgcaccagtgttgtctacaacaccactagattcacttggtctcaaaccttcaagaggtgtagaaagcttgcctctgccttgtcttctaggaacatctctagaggtgatgtcgtcagtgtccttgccccaaatgtcccagctatctacgagatgcatttcgccgtccctatggccggagctgtcttgaacaacgtcaacattagattggatgctagaactatggccgcccaattcacccattgtgaacctaagttcttgtttgtcgattaccagttccttccattggtcaccgaggccttgtctggaatccagcacaagcctagtgttgtcgtcatcgaggagcttcacaacggtagggaaatcgccacctctgccgctcttacttacgaaggtttggtcggtggaggagatccagacttcgaaattagatggccagaagacgagtggcaagctgccgccttgaactacacctctggtactacctctgccccaaagggtgtcgtccactgtcataggggtttgtacaccatggccatggacaatcttttgatgtggggtatgaccacccagccagttttcctttggacccttgccatgttccacgccaatggatggtgcttcccttggagtattgccgccgtcggaggaaccaatatctgcttgaggaaattcgacgccaagaccgtcttcgataagatcatcgaccataaggtcacccatttgtgcggagccccagttgtccttggtatgatggctaacgcccaccctagtgagagaaagccattgcccggtaaggtcgagatccttaccgctggtgctcctcctccagctgccatcttgtggaagatggaggagatgggtttcagtgtcacccacggttatggtttgaccgaaactgccggattggtcgttagttgcgcttggaagggagaatgggacaagttgcccggtaaggagagggccagattgaagagtaggcaaggtgttagaaacgtctctttggccgaagtcgacgttaaggatccagctaccatggcctctgttgctagagacggtcttcagatgggtgaggtcatgttgagaggtgccagtgtcatgaagggatacttgaagaacgaacagatgaccgctagggccatggatggtggttggtatagaaccggtgacgttggtgttttgcacccagacggttaccttgagatcaaggatagatctaaggacgtcatcattagtggtggtgaaaatattagttctgttgaggtcgaaagtgtcctttacacccacccattgatcgccgaggccgctgttgtcgctaaaccagatccattttggggtgagactccatgcgccttcgtctctcttaacaataatggtaccgagtgcttgagtgaggcccaagttatcagtttctgtagagagagaatcgcccacttcatggcccctaagagtgtcatcttcatgcaagaattgccaaaaacctctaccggaaagatccagaaattcgtccttagggagatggccaatagactttaa

SEQ ID NO: 26 - protein, 539 aa, Amentotaxus argotaenia
MEELKRCPANYPPLTPIGFIERAATVYGDCTSVVYNTTRFTWSQTFKRCRKLASALSSRNISRGDVVSVLAPNVPAIYEMHFAVPMAGAVLNNVNIRLDARTMAAQFTHCEPKFLFVDYQFLPLVTEALSGIQHKPSVVVIEELHNGREIATSAALTYEGLVGGGDPDFEIRWPEDEWQAAALNYTSGTTSAPKGVVHCHRGLYTMAMDNLLMWGMTTQPVFLWTLAMFHANGWCFPWSIAAVGGTNICLRKFDAKTVFDKIIDHKVTHLCGAPVVLGMMANAHPSERKPLPGKVEILTAGAPPPAAILWKMEEMGFSVTHGYGLTETAGLVVSCAWKGEWDKLPGKERARLKSRQGVRNVSLAEVDVKDPATMASVARDGLQMGEVMLRGASVMKGYLKNEQMTARAMDGGWYRTGDVGVLHPDGYLEIKDRSKDVIISGGENISSVEVESVLYTHPLIAEAAVVAKPDPFWGETPCAFVSLNNNGTECLSEAQVISFCRERIAHFMAPKSVIFMQELPKTSTGKIQKFVLREMANRL

SEQ ID NO: 27 - DNA, 1632 bp, Widdringtonia cedarbergensis
atggaggagcttaaaagatgcccagccaactaccctccacttacccctatcggattcgtcgaaagggccgccaccgtctacagtgactgcacctctgtcgtttacaacaccactagattcacttggtctcagaccttccaaaggtgcagaaagttggcctctgcccttgccttgagaaacatttgtagaggtgacgtcgtttctgttgttgctcctaacgtcccagctatgtacgaaatgcacttcgccgtccctatgtctggtgctgtccttaacaacttgaatactaggttggacgctagaaccatggctgcccagatctctcactgcgaaccaaaaatcatcttcgctgattatcaatttcttagtcttgtcaatgagaccttgagtcttcttaagcataaacctccacttgtcgtcatcgaggaaatgcagaacggtaggggtatcggtagaggtgccgagttgacctacgagggattgttgagagagggagatccagagttcgagattagatggccagaggacgaatggcaagctgccgtcttgaactacacctctggaaccacctctgctcctaagggagtcgttcaatctcatagaggtatctacgccatggccttggacaacttgaccatgtggcaaatgggtaggaggccaatctatctttggacccttgccatgttccacgccaatggatggtctcttccatggaccttggctgccgtcggaggtactaacatctgcttgaggaaatttgatgctaaaaccattttcgacagtatcgccgagcacaacgtcacccacatgtgcggagccccagttgtcttgtctatgatggccaacgccgatccagctgataggaaacaccttgcccatagagttgagatcttgaccgctggtgctccaccaccagctaccgtcttgtggaagatggaggagatgggattctctatcacccacggatacggacttaccgaaaccgccggtcttgttgtctcttgcgcttggaaaaccgaatgggatggtcttcccggtaaggagaaggctagacttaagtctaggcaaggagttaggaatcttagtttggccgaggtcgacgttaagaacccagttaccatggctagagtcaagagagacggagtccaaatgggtgaggtcatgatgaggggtgcctctatcatgaagggatacttgaagaatgatgacatcaccgctagagctatggagggtggttggtttagaaccggtgacgtcgctgtcattcacccagatggatatatcgaaattaaagatagatctaaagatgtcatcatttctggaggtgaaaacatttctagtgttgaagttgagtctgtcttgtacagtcaccctagtgtcgtcgaagctgccgtcgtcgccaaaccagactctttttggggtgagaccccatgcgcttttgtctctgtcaagaaccacggtggagagggtaatagagagatgttgtctgagggtgagatcatcgagttctgcagaaagcatcttgcccacttcatggccccaaaatctgtcatcattatgcaagagttgcctaagaccagtaccggtaaaattcaaaaattcgttttgagagaaatggctaaaaagctttaa

SEQ ID NO: 28 - protein, 543 aa, Widdringtonia cedarbergensis
MEELKRCPANYPPLTPIGFVERAATVYSDCTSVVYNTTRFTWSQTFQRCRKLASALALRNICRGDVVSVVAPNVPAMYEMHFAVPMSGAVLNNLNTRLDARTMAAQISHCEPKIIFADYQFLSLVNETLSLLKHKPPLVVIEEMQNGRGIGRGAELTYEGLLREGDPEFEIRWPEDEWQAAVLNYTSGTTSAPKGVVQSHRGIYAMALDNLTMWQMGRRPIYLWTLAMFHANGWSLPWTLAAVGGTNICLRKFDAKTIFDSIAEHNVTHMCGAPVVLSMMANADPADRKHLAHRVEILTAGAPPPATVLWKMEEMGFSITHGYGLTETAGLVVSCAWKTEWDGLPGKEKARLKSRQGVRNLSLAEVDVKNPVTMARVKRDGVQMGEVMMRGASIMKGYLKNDDITARAMEGGWFRTGDVAVIHPDGYIEIKDRSKDVIISGGENISSVEVESVLYSHPSVVEAAVVAKPDSFWGETPCAFVSVKNHGGEGNREMLSEGEIIEFCRKHLAHFMAPKSVIIMQELPKTSTGKIQKFVLREMAKKL

SEQ ID NO: 29 - DNA, 1629 bp, Cephalotaxus harringtonia
atggagaatatggaagagcttaagaggtgcgccgccaattatccacctttgacccctatcggattcatcgaaagggccgccactgtctacggagattgcaccagtgtcgtctacaacaccactagattcacttggtctcacaccttcttgaggtgtagaaagttggcctctgcccttagtagtaggagtatcagtagaggagacgtcgtcagtgttgttgcccctaacatcccagccatgtatgagatgcacttcgccgtcccaatggctggagccgtcttgaataacgtcaacattagacttgacgctaggactatggccgctcagttgaaccactgcaagccaaagttcgtcttcgtcgactaccagttcatgccattggttagagaggctcttaccgatcttgagcatagacctagtgtcgtcgttatcgaggagatggacaacggaagagaaattgccgccagtgccgccttgacctatgaagagttgatcaccgagggtaacccagaattcgagattaggtggccagaagatgagtgggaagctgccgttcttaactacaccagtggaaccacctctgctcctaaaggtgtcgtccactgtcatagaggtatttacgccatggccatggacaaccttcttatgtggggtatgaggacccagccagtcatcctttggaccttgccaatgttccacgccaatggttggtctgtcccttggagtgtcgctgccatgggtggaactaacatctgcgttagaaagttcgacgccaagatcgtcttcgacgcccttgccgagcataaggttacccatatgtgcggtgccccagtcgttttgtctatgatggccaacgcccagccatctgagagaaagcctcttcccggtaaggtcgagatccttaccgctggtgctcctcctccagctgccatcttgtggaagatggaagagatgggtttcagtgtcacccacggttacggtcttaccgagactgctggacttgtcgtcagttgcgcttggaaagccgagtgggacaaattgcccggtaaggaaagggctagattgaaggccagacaaggtgttaggaacgttaccttggccgaagtcgacgttaaagccccagccactatggcttctgtcgccagagatggtaggcagatgggagaggtcatgttgaggggagcctctgtcatgaaaggttacttgaaaaatgagcagatgaccgctagggccatggatggtggttggttcagaactggtgacgtcggtgtcatccacccagatggttaccttgaaatcaaggacagatctaaggatatcattatctctggaggtgagaacatcagttctgtcgaggtcgagtctgtcttgtactctcacccaatgattttggaagccgctgtcgttgctagaccagatccattttggggtgagactccatgcgctttcgtctctattaagaataaaagtaatgaagtcttgagtgaggcccaagttatctctttctgtagagaaaggatggcccacttcatggctcctaagtctgttattattatgcaagagttgccaaaaacttctactggaaaaatccagaagttcgttttgagagaaatggctaaatctctttaa

SEQ ID NO: 30 - protein, 542 aa, Cephalotaxus harringtonia
MENMEELKRCAANYPPLTPIGFIERAATVYGDCTSVVYNTTRFTWSHTFLRCRKLASALSSRSISRGDVVSVVAPNIPAMYEMHFAVPMAGAVLNNVNIRLDARTMAAQLNHCKPKFVFVDYQFMPLVREALTDLEHRPSVVVIEEMDNGREIAASAALTYEELITEGNPEFEIRWPEDEWEAAVLNYTSGTTSAPKGVVHCHRGIYAMAMDNLLMWGMRTQPVILWTLPMFHANGWSVPWSVAAMGGTNICVRKFDAKIVFDALAEHKVTHMCGAPVVLSMMANAQPSERKPLPGKVEILTAGAPPPAAILWKMEEMGFSVTHGYGLTETAGLVVSCAWKAEWDKLPGKERARLKARQGVRNVTLAEVDVKAPATMASVARDGRQMGEVMLRGASVMKGYLKNEQMTARAMDGGWFRTGDVGVIHPDGYLEIKDRSKDIIISGGENISSVEVESVLYSHPMILEAAVVARPDPFWGETPCAFVSIKNKSNEVLSEAQVISFCRERMAHFMAPKSVIIMQELPKTSTGKIQKFVLREMAKSL

SEQ ID NO: 31 - DNA, 1629 bp, Cephalotaxus harringtonia
atggaaaacatggaggagttgaagagatgcgccgccaactaccctcctttgacccctatcggattcatcgagagggccgccaccgtttatggtgactgcacttctgtcgtctacaacaccactagattcacttggtctcacaccttcttgaggtgcagaaagcttgcctctgctttgagttctaggtctatcagtagaggtgacgtcgtcagtgttgttgctcctaacatcccagctatgtacgaaatgcacttcgccgtcccaatggctggagccgtcttgaataacgtcaacattagacttgacgctaggaccatggccgctcagttgaaccactgcaaaccaaagttcgtctttgtcgactaccagtttatgccattggttagagaggccttgaccgatcttgaacatagaccatctgtcgtcgtcatcgaggagatggacaacggtagagagatcgctgcctctgctgccttgacctatgaggagttgatcaccgagggtaacccagagttcgagatccaatggccagaagacgagtgggaagctgccgtcttgaactacacctctggtaccaccagtgctcctaagggtgtcgtccactgtcatagaggtatttacgccatggccatggacaaccttcttatgtggggtatgaggacccagccagtcatcttgtggaccttgcctatgttccacgccaatggttggtctgtcccttggagtgtcgctgccatgggtggaactaacatctgcgttagaaagttcgacgccaagatcgtcttcgacgcccttgctgagcacaaagtcacccatatgtgtggtgccccagtcgtcttgtctatgatggccaatgcccagccatctgaaaggaagcctcttcccggtaaggtcgagatccttaccgctggagctcctccaccagctgccatcttgtggaaaatggaggagatgggtttttctgtcacccacggttacggtcttaccgagactgctggacttgtcgtctcttgtgcttggaaggccgagtgggataaattgcccggtaaggagagggctaggcttaaagctagacaaggagttaggaacgtcacccttgccgaagtcgatgtcaaggatccagctactatggcctctgttgccagagatggtaggcagatgggagaggtcatgttgaggggagcctctgtcatgaagggttatcttaaaaacgaacagatgactgccagagccatggatggtggatggtttagaactggagatgtcggtgtcattcatccagacggttacttggagatcaaggacagaagtaaggatattatcattagtggtggagagaacatcagttctgtcgaggtcgagtctgtcttgtatagtcacccaatgatccttgaagccgctgtcgttgctagaccagatccattctggggagagaccccttgcgctttcgtctctatcaagaacaaaagtaacgaagttttgagtgaggcccaagttatctctttctgtagagaaaggatggcccacttcatggctcctaagtctgttattattatgcaagagttgccaaaaacttctaccggaaaaatccagaagttcgtccttagggaaatggccaagagtttgtaa

SEQ ID NO: 32 - protein, 542 aa, Cephalotaxus harringtonia
MENMEELKRCAANYPPLTPIGFIERAATVYGDCTSVVYNTTRFTWSHTFLRCRKLASALSSRSISRGDVVSVVAPNIPAMYEMHFAVPMAGAVLNNVNIRLDARTMAAQLNHCKPKFVFVDYQFMPLVREALTDLEHRPSVVVIEEMDNGREIAASAALTYEELITEGNPEFEIQWPEDEWEAAVLNYTSGTTSAPKGVVHCHRGIYAMAMDNLLMWGMRTQPVILWTLPMFHANGWSVPWSVAAMGGTNICVRKFDAKIVFDALAEHKVTHMCGAPVVLSMMANAQPSERKPLPGKVEILTAGAPPPAAILWKMEEMGFSVTHGYGLTETAGLVVSCAWKAEWDKLPGKERARLKARQGVRNVTLAEVDVKDPATMASVARDGRQMGEVMLRGASVMKGYLKNEQMTARAMDGGWFRTGDVGVIHPDGYLEIKDRSKDIIISGGENISSVEVESVLYSHPMILEAAVVARPDPFWGETPCAFVSIKNKSNEVLSEAQVISFCRERMAHFMAPKSVIIMQELPKTSTGKIQKFVLREMAKSL

SEQ ID NO: 33 - DNA, 1632 bp, Prumnopitys andina
atggaagaattgaaaaggtgcccagctaaccaccctccattgacccctatcggattcattgagagggcttctaccgtctacggtgactgcatctctgttgtctacaacacctctagatatacttggagtcaaacctttcagaggtgctgcaagcttgcttctgccttgtgcagtaggaagatcagtagaggagacgtcgtttgcgttgttgctcctaacatcccagccatgtacgaaatgcactttgccgtccctatggccggtgccgtccttaacaatgtcaaccatagacttgacgctagaactatgggagcccagtttaggcactgcgagcctaagttccttttcgtcgactaccagttccttagaccagtcagtgaggccttggaggagatcgagcacaaaaagcctaccgtcgtcgtcattgaagaggtcgaggaaggtaaagaagttccatctagtggagctgctttgacctacgagggtttggtcgaggagggagaacccggtttcgaaactagatggccagaagacgagttccaagctgctgccttgaactataccagtggtaccacctctgaccctaaaggtgtcgtccactctcatagaggtttgtacaccatggctatggataatttgcttatttggggtatgaagtctcagccagttttcttgtggaccttgcctatgttccacgctaatggatggtgcttcccttggtctcttgccgctgtcggaggtaccaatatctgcttgaggaaattcgatgccaaaattgtctttgacgccttggccgaacacggtgttactcatttgtgcggtgccccagttgtccttaatcttattgccaacgcccatccatctgagaggaggcctcttccagccagagttgaagttcttaccgctggatctcctcctccagctgccatcttgttgaagatggacgaattgggtttttctgtcacccacggatacggattgactgaaactgccggtttggtcgtctcttgcgcttggaagactaagtgggacgaacttccagctagagacagagctaggttgaaggctaggcaaggtgttaggaccctttctcttgccgaggttgacgtcaaggatccagataccatggccagtgttcctagagacggtcagtctatgggtgaggtcatgttgaggggtactagtgtcatgaagggttatcttaagaatagagccatgaccgctagaactttggccggtggttggtttcataccggtgacgtcggtgttgtccacccagacggttaccttgaaatcaaggatagaagtaaagatattatcatcagtggtggtgagaatatctcttctgttgaagttgaatctgttctttactctcatccattggttgttgaggccgctgttgtcgctagaccagatagtttctggggagagactccatgcgctttcgtcagtgttatgaactctgttacccagaccagtcttgaggccgagatcatcagtttctgtagagagagattgcctcacttcatggcccctaaaagtgtcgttttccttaaagaacttcctaagaccagtaccggaaaggttcagaagttcgccttgagggagatggccaagagattgccaaaataa

SEQ ID NO: 34 - protein, 543 aa, Prumnopitys andina
MEELKRCPANHPPLTPIGFIERASTVYGDCISVVYNTSRYTWSQTFQRCCKLASALCSRKISRGDVVCVVAPNIPAMYEMHFAVPMAGAVLNNVNHRLDARTMGAQFRHCEPKFLFVDYQFLRPVSEALEEIEHKKPTVVVIEEVEEGKEVPSSGAALTYEGLVEEGEPGFETRWPEDEFQAAALNYTSGTTSDPKGVVHSHRGLYTMAMDNLLIWGMKSQPVFLWTLPMFHANGWCFPWSLAAVGGTNICLRKFDAKIVFDALAEHGVTHLCGAPVVLNLIANAHPSERRPLPARVEVLTAGSPPPAAILLKMDELGFSVTHGYGLTETAGLVVSCAWKTKWDELPARDRARLKARQGVRTLSLAEVDVKDPDTMASVPRDGQSMGEVMLRGTSVMKGYLKNRAMTARTLAGGWFHTGDVGVVHPDGYLEIKDRSKDIIISGGENISSVEVESVLYSHPLVVEAAVVARPDSFWGETPCAFVSVMNSVTQTSLEAEIISFCRERLPHFMAPKSVVFLKELPKTSTGKVQKFALREMAKRLPK

SEQ ID NO: 35 - DNA, 1632 bp, Taxus x media
atggaggatttgaaaaggtgcccagctaactaccctccacttacccctatcggtttcatcgagagagccgccactgtctatagagattgcaccagtttggtctacaacaccactagatttacttggagtcagactttcaatagatgccttaaaatggcctctgccttgtctagtaggaacatctctaggggagacgtcgtctctgttgtcgcccctaacgtcccagctatttacgagatgcacttcgctgtcccaatggccggagccgtcttgaacaacgtcaacattagacttgatgccagaaccatggctactcaatttatccattgtgaaccaaaattcgtttttgttgattaccagttccttccattggttagagaggccttgagtgagatcggtaacaagccatgcgtcgttgttatcgaggagcttgacaacggtagggaaattgccaccagtaccggtttgacttacgaaggaatgatcggagagggagacccaaagttcgaaattagatggccagaggacgaatggcaagccgccgtccttaattacaccagtggtaccacctctgccccaaagggagttgtccattgccatagaggactttactctatggccatggacaacttgttgatgtggggacttaccactagaccagtttacttgtggaccttggccatgttccacgccaatggatggtgtttcccatggactcttgccgccgtcggtggtaccaatatctgtttgagaaaattcgacgccaaaatcattttcgacgctttggccgagcacggtgtcactcatatgtgcgctgccccagttgtcttgtctatgatgcttaacgcccacccatctgagagaaagccaatggctaatagagtcgagatccttaccgccggttctcctcctccagccgctattttgagaaagggagaggagatgggttttttcgtcacccacggttacggtttgactgaaaccgccggattggtcgtcagttgtgcttggaaagctgagtgggacaacctttctgccgaggaaagagctaggttgaaagctagacaaggtgttagaaacttgagtttgaccgaggtcgacgttaaggatccagcttctatggcttctgttgccagagacggtgtccaaatcggtgaagtcatgttgaggggagcctctgttatgaagggttacttgaaaaacgagcagatgaccgcccaagctatggacggtggatggtttcatactggtgacgtcgccgttcttcacccagacggttatcttgaaattaaggatagaagtaaggacattattatctctggaggtgagaacatctcttctgtcgaagtcgagtctgtcctttacagtcatcctttgatcatggaggccgccgttgttgctagaccagatccattctggggtgagaccccatgtgcttttgtctctatcaataacgatggtaaagaggtccttagtgaggcccagatcatctctttctgcaaggagaggactgcccatttcatggctcctaagtctgtcatctttatgaaagaattgcctaagacctctaccggaaaggtccagaaattcgttcttagggaaatggccaaaaacttggctcagcctaggtaa

SEQ ID NO: 36 - protein, 543 aa, Taxus x media
MEDLKRCPANYPPLTPIGFIERAATVYRDCTSLVYNTTRFTWSQTFNRCLKMASALSSRNISRGDVVSVVAPNVPAIYEMHFAVPMAGAVLNNVNIRLDARTMATQFIHCEPKFVFVDYQFLPLVREALSEIGNKPCVVVIEELDNGREIATSTGLTYEGMIGEGDPKFEIRWPEDEWQAAVLNYTSGTTSAPKGVVHCHRGLYSMAMDNLLMWGLTTRPVYLWTLAMFHANGWCFPWTLAAVGGTNICLRKFDAKIIFDALAEHGVTHMCAAPVVLSMMLNAHPSERKPMANRVEILTAGSPPPAAILRKGEEMGFFVTHGYGLTETAGLVVSCAWKAEWDNLSAEERARLKARQGVRNLSLTEVDVKDPASMASVARDGVQIGEVMLRGASVMKGYLKNEQMTAQAMDGGWFHTGDVAVLHPDGYLEIKDRSKDIIISGGENISSVEVESVLYSHPLIMEAAVVARPDPFWGETPCAFVSINNDGKEVLSEAQIISFCKERTAHFMAPKSVIFMKELPKTSTGKVQKFVLREMAKNLAQPR

SEQ ID NO: 37 - DNA, 1632 bp, Microcachrys tetragona
atggaaggtttgaaaaggtgcccagctaaccaccctccacttacccctatcggtttcatcgagagggcttctaccgtctacggtgactctatcagtcttgtctacaatacctctcactacacttggtctcaaaccttccagaggtgtaggaagcttgctagtgccctttgctctagaaagatcagtaggggagatgtcgtcagtgtcgtcgcccctaacactccagctatctacgaaatgcacttcgccgtccctatggctggtgctgtccttaacaacgtcaatactagacttgacgctagaaccatgggtgctcagtttaggcactgcgagcctaagttcgtcttcgtcgactaccagttgcttaacttggtcagtgaagccgtcgctaccattgaggacaacaagccaaccgttgtcgtcatcgaagagttggagcacggtaaggaagtcttgagttctggagtcgccttgacctacgaacagcttgttgaggagggtcaacccggtttcgagacccaatggccagatgacgagtgggaagccgccgtcttgaactacaccagtggaaccacctctgaccctaaaggtgtcgtccattctcatagaggtttgtacaccatggcccttgataatttgcttatttggggtatgaagtctcagccagtctttttgtggaccttggccatgttccacgctaatggatggtgctttccttgggcccttgccgccgtcggaggaaccaacatctgccttaggaagtttgacgccaagatcgtctttgatgcccttgctgagcatggagtcactcatttgtgcggtgccccagttgtcttgtctatgatcgccaacagtcacccttgtgatagacaacctcttcacggaagggtcgaagttttgaccgctggttctcctcctccagctcctatcttgagtaagattgagggtttgggtttctctgtcacccacggttacggattgaccgaaactgccggacttgtcgttagttgcgcttggaagaccaagtggaacaagttgccaccagaagatagggctagacttaaggctaggcaaggagtcagaatccttagtcttgccgagttcgacgtcaaggacccagcttctatggcctctgttccaagggacggtcaaagtatgggtgaggtcatgcttaggggttctagtatcatgaagggatatcttaagaagccagagatgaccgctagagctcttgaaggtggttggttgcacactggagatgtcggagttgtccacccagacggttaccttgagattaaggataggtctaaggatatcatcatttctggaggtgaaaatatcagtagtgtcgaagttgaaagtatcctttattctcacccattggttgttgaagccgccgttgtcgctagaccagataacttctggggtgagaccccttgcgctttcgtcagtttgaacaccaaaaaggccgagaggtctcttgaagtcgagatgatcagtttctgtagagagagattgcctcacttcatggctccaaagtctgtcgttcttatgaaagagttgccaaagacctctactggaaaaatccaaaaattcgtcttgagggagatggctaagaatcttcctaactaa

SEQ ID NO: 38 - protein, 543 aa, Microcachrys tetragona
MEGLKRCPANHPPLTPIGFIERASTVYGDSISLVYNTSHYTWSQTFQRCRKLASALCSRKISRGDVVSVVAPNTPAIYEMHFAVPMAGAVLNNVNTRLDARTMGAQFRHCEPKFVFVDYQLLNLVSEAVATIEDNKPTVVVIEELEHGKEVLSSGVALTYEQLVEEGQPGFETQWPDDEWEAAVLNYTSGTTSDPKGVVHSHRGLYTMALDNLLIWGMKSQPVFLWTLAMFHANGWCFPWALAAVGGTNICLRKFDAKIVFDALAEHGVTHLCGAPVVLSMIANSHPCDRQPLHGRVEVLTAGSPPPAPILSKIEGLGFSVTHGYGLTETAGLVVSCAWKTKWNKLPPEDRARLKARQGVRILSLAEFDVKDPASMASVPRDGQSMGEVMLRGSSIMKGYLKKPEMTARALEGGWLHTGDVGVVHPDGYLEIKDRSKDIIISGGENISSVEVESILYSHPLVVEAAVVARPDNFWGETPCAFVSLNTKKAERSLEVEMISFCRERLPHFMAPKSVVLMKELPKTSTGKIQKFVLREMAKNLPN

SEQ ID NO: 39 - DNA, 1596 bp, Athrotaxis cupressoides
atgttgaacccatcttctggattcagtttggccaccggaatctactactctaagagggacccagctccattgcctccaccaaggcaacacttggacatcaccacttacgtcttcagtcaccctcacaacaccgaaatcgccatcattgacgccaagagtgacgcccagttgtcttatagagccttgaggcacaacgttagagctttggctaccggtcttcagaggttgggtatcagaaagagggacgtcgtcttggtcattcttccaaacattattcatgtcccatctatctaccttgccaccgtcagtatcggtgctatcttgaccaccgccaatccattgaacaccgaggccgagatccaaagacaagttgctgacagtaatccagtccttgctatcgtcgctccagaatttgctcacaaggctagagccgctcaattgccagtcgtcttgacccaaaccacctctccaaccgctaatcagtctggttacgtcgccaccttgcacgagcttttccagtctaacgttgacgacttccagagtgtcgatgtccagcaagaagacaccgctaccttgttgtactctagtggtaccaccggtaagtctaagggagtcgtcgctactcacagaaatcacatcgccatggtcgccggtttcgttcagaggatcgactctgagaagatcgtctctttgtgcaccatgccacttttccatgtttacggtttgttctactctatcatcgccgtcgccaccggtaccaccttgatcttgatggctaagttcgatttcgccgagatgttggccaacgtcgagaggtacaaggttgtctctcttccagtcgctcctccaattttcgtcgccttgaccaagtctcctattgttgccaaatatgacttgtcttctcttaagaggatcggttctggtggtgctgcccttggtaaggaaatcatcgacgagttcaccgccctttatccaaatatcgaggttagtcaaggttacggattgaccgagagttctggtgccgttaccttcactagtaccggagaggagaagaagaagtacggaaccgctggtttgttggccgccaacttggaggccaagatcgtcgataccgtctctgaaaaggccttgccaccaaaccagaggggtgaattgtggttgaggggtccaaccgtcatgaagggttacttctgcaacgctgaggccaccgctactaccttggacagtgagggttggcttaagaccggtgatttgtgctacttcgacgaggagggtttccttttcgtcgtcgacagaatcaaggagttgatcaagtacaagggttatcaagtcgccccagccgagttggaagaattgttgttgtctaacccacagatttctgacgccgctgttatcccatacccagacaaagaggctggtcagatcccaatggctttcatcgttagaaaggccgacagtaagcttaaggaagaggacgttaagtctttcgtctctaagcaagttgccccttataagaagattagaagggtcgcctttgttccatctatcccaaagagtgccagtggtaagattttgaggaaggatttgattcaataa

SEQ ID NO: 40 - protein, 531 aa, Athrotaxis cupressoides
MLNPSSGFSLATGIYYSKRDPAPLPPPRQHLDITTYVFSHPHNTEIAIIDAKSDAQLSYRALRHNVRALATGLQRLGIRKRDVVLVILPNIIHVPSIYLATVSIGAILTTANPLNTEAEIQRQVADSNPVLAIVAPEFAHKARAAQLPVVLTQTTSPTANQSGYVATLHELFQSNVDDFQSVDVQQEDTATLLYSSGTTGKSKGVVATHRNHIAMVAGFVQRIDSEKIVSLCTMPLFHVYGLFYSIIAVATGTTLILMAKFDFAEMLANVERYKVVSLPVAPPIFVALTKSPIVAKYDLSSLKRIGSGGAALGKEIIDEFTALYPNIEVSQGYGLTESSGAVTFTSTGEEKKKYGTAGLLAANLEAKIVDTVSEKALPPNQRGELWLRGPTVMKGYFCNAEATATTLDSEGWLKTGDLCYFDEEGFLFVVDRIKELIKYKGYQVAPAELEELLLSNPQISDAAVIPYPDKEAGQIPMAFIVRKADSKLKEEDVKSFVSKQVAPYKKIRRVAFVPSIPKSASGKILRKDLIQ

SEQ ID NO: 41 - DNA, 1632 bp, Larix speciosa
atgatcatcgaccctcagtctggttactgtagagaaactggaatctactactctaaaagggaccctatcgacttgttgcctccaacccagcatttggacgccaccacttacatcttctctcatcacaaccatacccaagaaattgccttcatcgacgcttctagttctttgtctcttagttacccagctcttaggcacaatgttagagctttggctgccggtatgcacggacttggtatcagaaagggaaatgttgtcttggtcatcagtcctaattctatcgccttgccttgcatctacttggccatcttgtctatcggtgccatccttagtaccgccaaccctttgaataccgaggccgaaatcaagaagcaagctgaggatagtaagccagtcatcatcttcaccccagctaaccttattaacaaggttagagccacccagttgccagtcatccttatcgagggtaacaccgaggaaggaagtggatctggttctggttgtattagtactcttaatcttttgttgcaaagtgatattagtggtttcccaagtgtcgatatcaagcaagaagacaccgccaccttgttgtacagtagtggaaccaccggtaaaagtaagggagtcatctctacccatagaaacttgatcgctatgatcgccggtgttttgagtactgctgccgacatcgagggagaggagaaaatctctctttgcatcatccctctttttcatgtttttggattcttttatacccttttctgcgtcgccgctggtttgaccatggtcgtcatgccaaagttcggtttcgccgagatgttgtctgccattcagcaatataaggtcaagagtttgccagcctctccaccaatccttgtcgccttgaacaagagtccaatcgttgctaattatgatttgtctagtttgtactctatcgcctctggtggtgccccattgggtaaggacgtcatcgacaatttcaccgctagatttccaactgtccaagttcagcaaggttacggtttgaccgagtctagtggttctgtcagttctaccaccaccgaagaggaaaatagacactatggtaccgccggtcttttggctccaaacgtcgaagctaaggtcgtcgataccgcttctggtaagaccatgccacctaactacaagggagagttgtggttgaggggtccaaccatcatgaagggatacttcggtaatgacgaggccactgcctctaccttggactctgagggttggttgaagaccggtgacttgtgttatattgatgaagagggattcttgttcgtcgtcgatagagttaaggagcttatcaaatacaaggccttccaagttgccccagccgagcttgaagagcttttgttgtctaatccagagatctctgacgccgccgttattccttacccagatgacgaagccggtcagatcccaatggccttcgtcgccagaaagagtgactctaacctttctgaacaagatgttattaatttcgtcgccaaacaagtctctccatataagaaaattagaagggtcgccttcgtcaatagtatcccaaagtctccatctggtaaaatcttgagaaaggaccttatccaccaagctctttctacctaa

SEQ ID NO: 42 - protein, 543 aa, Larix speciosa
MIIDPQSGYCRETGIYYSKRDPIDLLPPTQHLDATTYIFSHHNHTQEIAFIDASSSLSLSYPALRHNVRALAAGMHGLGIRKGNVVLVISPNSIALPCIYLAILSIGAILSTANPLNTEAEIKKQAEDSKPVIIFTPANLINKVRATQLPVILIEGNTEEGSGSGSGCISTLNLLLQSDISGFPSVDIKQEDTATLLYSSGTTGKSKGVISTHRNLIAMIAGVLSTAADIEGEEKISLCIIPLFHVFGFFYTLFCVAAGLTMVVMPKFGFAEMLSAIQQYKVKSLPASPPILVALNKSPIVANYDLSSLYSIASGGAPLGKDVIDNFTARFPTVQVQQGYGLTESSGSVSSTTTEEENRHYGTAGLLAPNVEAKVVDTASGKTMPPNYKGELWLRGPTIMKGYFGNDEATASTLDSEGWLKTGDLCYIDEEGFLFVVDRVKELIKYKAFQVAPAELEELLLSNPEISDAAVIPYPDDEAGQIPMAFVARKSDSNLSEQDVINFVAKQVSPYKKIRRVAFVNSIPKSPSGKILRKDLIHQALST

SEQ ID NO: 43 - DNA, 1641 bp, Austrotaxus spicata
atgcagaagatggaagaccttaagaggtgcccagccaactacccaccacttaccccaatcggattcatcgagagagccgccaccgtctacggagactgcaccagtcttgtctacaacaccactaggttcacttggtctcagaccttcaatagatgcttgaagttggccagtgccctttctagtaggaacatcagtaggggtgacgtcgtttctgttgccgccccaaacgtcccagctatgtacgagatgcacttcgccgttccaatggctggtgccgtcttgaacaccgtcaacattagacttgacgctagaaccatggccgctcagttcactcactgcgagcctaagttccttttcgtcgattatcaattcttgccagtcgtcagtgaggcccttagtaggattggtcacaaaccatgcgtcgtcgtcatcgaagaacttgacaacggaagggagatcgctacctctgccggacttacctatgagggacttatcggtgagggtgacaccgagttcgagattagatggccagaagacgaatggcaagctgctgtcttgaactacacctctggtaccactagtgcccctaaaggtgtcgtccactgtcatagaggaatctacaccatggccatggataaccttttgatgtggggtttgaggactaggccagtttacttgtggaccttggctttgttccacgccaatggttggtgtttcccttggagtttggccgccatgggaggtactaacatctgtttgaggaagttcgatgccaagatcatcttcgacgccttggccgaacatggagtcacccatatgtgcgccgctccagtcgtcttgtctatgatcgtcaacgcccacccatctgagaggaggccagttgctggaagagttgagatcttgaccgctggatctccaccaccagctgctatcttgggaaaggtcgagcaaatgggtttcgccgtcactcatggatacggtttgagtgagaccgctggtttggttgtcagttgcgcttggaaggctgaatgggataacttgccagccgaagagagggccagattgaagtctaggcaaggtgttagaaacgttagtttggccgaagtcgacgtcaaagacccaacctctatggcctctgttgccagagacggtgttcaaatcggtgaggtcatgatgaggggtgccagtgtcatgaagggttatttgaaaaaccaagagatgaccgctagggccatggatggtggatggtttaggaccggagacgttggtgttcttcacccagatggataccttgagatcaaggatagatctaaagatatcatcatcagtggtggagagaacatctcttctgtcgaggtcgagagtgtcctttactctcaccctcttattgtcgaagctgccgtcgttgctagaccagatccattctggggtgagaccccatgcgccttcgtctctatcaacaacaactctaagcaagctttgtctgaggcccaagttatcagtttctgtagagagagaatgccacacttcatggccccaaagagtgttatctttatgaaagaccttccaaaaacttctactggtaagatccaaaagtttattttgagggatatggctaagaacttggcccagccaagataa

SEQ ID NO: 44 - protein, 546 aa, Austrotaxus spicata
MQKMEDLKRCPANYPPLTPIGFIERAATVYGDCTSLVYNTTRFTWSQTFNRCLKLASALSSRNISRGDVVSVAAPNVPAMYEMHFAVPMAGAVLNTVNIRLDARTMAAQFTHCEPKFLFVDYQFLPVVSEALSRIGHKPCVVVIEELDNGREIATSAGLTYEGLIGEGDTEFEIRWPEDEWQAAVLNYTSGTTSAPKGVVHCHRGIYTMAMDNLLMWGLRTRPVYLWTLALFHANGWCFPWSLAAMGGTNICLRKFDAKIIFDALAEHGVTHMCAAPVVLSMIVNAHPSERRPVAGRVEILTAGSPPPAAILGKVEQMGFAVTHGYGLSETAGLVVSCAWKAEWDNLPAEERARLKSRQGVRNVSLAEVDVKDPTSMASVARDGVQIGEVMMRGASVMKGYLKNQEMTARAMDGGWFRTGDVGVLHPDGYLEIKDRSKDIIISGGENISSVEVESVLYSHPLIVEAAVVARPDPFWGETPCAFVSINNNSKQALSEAQVISFCRERMPHFMAPKSVIFMKDLPKTSTGKIQKFILRDMAKNLAQPR

SEQ ID NO: 45 - DNA, 1632 bp, Halocarpus bidwillii
atggaggaattgaaaaggtgcccagctaaccaccctccacttacccctatcggtttcatcgagagagctagtgtcgtctacggagactgcaccagtgttgtctacaactcttctaggtacacttggtctcagacctttcagaggtgctgcaagcttgctagtgctttgtgcagtaggaacatccttaggggagacgtcgttagtgtcgtcgctcctaacattccagccctttacgaaatgcatttcgctgtccctatgaccggagccgtccttaacaacgttaacactagattggatgctagaaccatgggagcccagtttaggcactgcgagcctaagttcgtcttcgccgactaccagcttttgccacttgtcagtgaggccttggctagaatcgagcacaacaagccaaccgttgtcgtcatcgaagagttggagcacggtaaggaagtcgtcagtagttgcagaaccttgacctacgagaaattgcttgaggaggccgaacccggtttcgaaatccagtggccagatgacgaatggcaagctgctgtcttgaactacaccagtggtaccactagtgaccctaaaggagtcgtccactctcatagaggtttgtacaccatcagtatcgacaaccttgtcatgtggggtatgaggtctcagccagttttcttgtggaccttgccaatgttccacgccaacggttggtgcttcccttgggctttggctgccgttggtggtaccaacatctgcttgaggaagttcgacgccaagatcgtcttcgatgctttggccgagcatggagttacccacttgtgcggtgccccagttgttctttctatgatcgccaacgcccagccttctgagaggaagtctcttcccggtagggtcgaagtccttaccgctggttctccaccaccagccactatcttgtggaagatggacgaattgggtttctctgtcacccactgctacggtttgaccgaaaccgccggtttggccgtctcttgcgcttggaagagtaagtggaacaagcttccagctgaagatagagccagattgaaggctaggcaaggagtcagagtcctttctcttgctgccgttgacgtcaaggatccagcttctatggtcagtgtccctagagacggtcagaccatgggtgaggttatgttgaggggtacctctttgatgaagggttaccttaagaacccagatatgaccgctagggcccttgagggaggatggttccacaccggagatatgggtgtcgttcacccagatggttaccttgaaatcaaggatagaagtaaagatattatcattagtggtggtgagaatatctcttctgttgaagttgaatctattttgtactctcatccattggttgttgaagccgccgttgtcgctagaccagatccattctggggtgagaccccatgtgccttcgtcagtttgagtaccaacagtggtcagcctagtcttgagaaggatatcatttctttctgtagagagaggttgcctcactttatggccccaaagagtgttgtctttattaaagaattgccaaagaccagtactggtaaaatccagaaattcgtcttgagggagatggccaaatctttgcctaagtaa

SEQ ID NO: 46 - protein, 543 aa, Halocarpus bidwillii
MEELKRCPANHPPLTPIGFIERASVVYGDCTSVVYNSSRYTWSQTFQRCCKLASALCSRNILRGDVVSVVAPNIPALYEMHFAVPMTGAVLNNVNTRLDARTMGAQFRHCEPKFVFADYQLLPLVSEALARIEHNKPTVVVIEELEHGKEVVSSCRTLTYEKLLEEAEPGFEIQWPDDEWQAAVLNYTSGTTSDPKGVVHSHRGLYTISIDNLVMWGMRSQPVFLWTLPMFHANGWCFPWALAAVGGTNICLRKFDAKIVFDALAEHGVTHLCGAPVVLSMIANAQPSERKSLPGRVEVLTAGSPPPATILWKMDELGFSVTHCYGLTETAGLAVSCAWKSKWNKLPAEDRARLKARQGVRVLSLAAVDVKDPASMVSVPRDGQTMGEVMLRGTSLMKGYLKNPDMTARALEGGWFHTGDMGVVHPDGYLEIKDRSKDIIISGGENISSVEVESILYSHPLVVEAAVVARPDPFWGETPCAFVSLSTNSGQPSLEKDIISFCRERLPHFMAPKSVVFIKELPKTSTGKIQKFVLREMAKSLPK

SEQ ID NO: 47 - DNA, 1605 bp, Taiwania cryptomerioides
atggcctctgacttggatccatctagtggtttctctagggccaccgagatctactattctaagagggacccagccccattgccacctcttcaccagcatcttgacttgaccacctacgtcttcagtcaccctcataacaccgacaccgcctttatcgacgcccaatctggtacccaattgagttacaccgctttgagacataacgttagagctcttgccaccggacttcagaggttgggtattagaaaaagggatgttgttttgcttatttctccaaataatatccacgttccttgcatctacttggccattgtctctatcggagccatcttgaccaccgctaacccacttaacaccgaagccgagattaggaggcaagttgtcgactctaacccagtcttggctttcgctgccccagagtttgctcacaaagccagagctgcccaacttccagtcgtccttacccagaagacctctccaatcgctaacgattctggttacgtcgctaccttgcacgagctttttcagagtaacgttgacgactttcagagtgtcgatatccagcaaggtgacaccgccaccttgctttactctagtggaaccaccggtaagaataagggagtcgttgccacccatagaaatcacatcgccatggtcgctggaaccttgcaaaggatcgacccaattaagcacattactttgtgcatcgtcccattgttccatgtctatggtttcttctacactttgtctgccgtcgccaaaggagctacccttactttcatgaccaagttcgacttcgcccagatgcttgccaacgtcgctaggtatagaatcaccagtcttgctatcgctcctcctatcttcgttgcccttaccaagtctccaatcgtcgccaagtacgatctttcttctttgaaaagaatcggttctggtggtgccgcccttggaaaggagactatcgacgaattcatcgctttgtttccaaacatcgaagtctctcaaggttacggacttaccgagagtagtggtgccgtcaccttcacctctactggagaggagaagaagaagtacggtaccgccggtcttttggccgctaacgtcgaagccaaaatcgtcgacatcatttctggtaaggctttgcctccaaaccaaaggggtgagctttggttgaggggaccaaccatcatgaagggatacttcttgaacgccgaagccaccgctactaccttggactctgagggatggttgaagaccggtgacctttgctacttcgacgaggaaggttttctttttattgttgatagaattaaggaacttatcaagtacaaaggttatcaagttgccccagccgagcttgaagagcttttgctttctaactctgagattgttgacgccgccgtcattccttacccagataaggaggctggacagattccaatggccttcatcgttagaaaggctgacagtaagttgaccgaggaggacgtcaagagttacgtcagtaagcaagttgccccatacaaaaagatcagaagggttgccttcgtcacctctatccctaagtctgctagtggtaaaatcttgaggaaagatttgatccaataa

SEQ ID NO: 48 - protein, 534 aa, Taiwania cryptomerioides
MASDLDPSSGFSRATEIYYSKRDPAPLPPLHQHLDLTTYVFSHPHNTDTAFIDAQSGTQLSYTALRHNVRALATGLQRLGIRKRDVVLLISPNNIHVPCIYLAIVSIGAILTTANPLNTEAEIRRQVVDSNPVLAFAAPEFAHKARAAQLPVVLTQKTSPIANDSGYVATLHELFQSNVDDFQSVDIQQGDTATLLYSSGTTGKNKGVVATHRNHIAMVAGTLQRIDPIKHITLCIVPLFHVYGFFYTLSAVAKGATLTFMTKFDFAQMLANVARYRITSLAIAPPIFVALTKSPIVAKYDLSSLKRIGSGGAALGKETIDEFIALFPNIEVSQGYGLTESSGAVTFTSTGEEKKKYGTAGLLAANVEAKIVDIISGKALPPNQRGELWLRGPTIMKGYFLNAEATATTLDSEGWLKTGDLCYFDEEGFLFIVDRIKELIKYKGYQVAPAELEELLLSNSEIVDAAVIPYPDKEAGQIPMAFIVRKADSKLTEEDVKSYVSKQVAPYKKIRRVAFVTSIPKSASGKILRKDLIQ

SEQ ID NO: 49 - DNA, 1542 bp, Dacrycarpus compactus
atgagtgacttgaatcagaagggtggttactgcgctgccaccggtatttaccacagtttgagggagccattgccacttcctccaccatatcagaaccttgacatcaccacctttgtcttcagtcaccaccacaccactgacattgccttgatcgacgcccacactggttgtagtttgagttacgccgccttgaggagaaacgtcaagtctttgaccgccgccttgcaccacttgggaatcggtaagggtgacgtcgtcttggttctttcttctaatagtatccacatcccatgcatctacatggccatcttctctgtcggtgccatccttaccaccgccaatccattgaatacccagagtgagatccagattcaagttaccgagagtaacccagctatcatcttcgccgctaccgatcttgttccaaaggctagagccactaacagaccagtcgtcgtcatcgacaaagagaaggaggagcagcatggatgcgtcgccaccttgtggaagcttttgcaaagttctgtcaatgaagctagaaaacttgacattcaccaagatgacaccgccaccttgttgtattcttctggaaccaccggtaagtctaagggtgtcgtcggtagtcatagaaatttcatctctatggtcgccggtcttgtcagtggtgccaacgagtggaactctgtcgtcatgatgaccatgccaatgttccacgtctacggattccttttcgccatgtctttccttgccagtggttctaccgtcgtcgtcatgccaaaatttgacttcgccttgatgctttggtctgtccagaggtatagagtcaagtacttgccaaccagtccaccattgttcgtcgctcttaccaagagtccaatggttgaaaaatacgacttgagttctttggatagagtcgtcagtggaggtgctccattgggtaaagaagttatcgacgagtttgccaacagattcccagatgttgaggttgcccaaggttatggtttgaccgagagttgtggtgccgtcaccttcaccagttctgccgaggagaagaagaagtatggtaccgccggtttgttggccgctaacatggaggccaagatcgtcgacaccgttaccggaaaagccttgccacctaatcagaggggtgagttgtggttgagaggtccttctattatgaaaggttactacaataacgaggaggccaccgccgccaccttggactctgaaggatggcttaagaccggagacttgtgctacatcgatgacgagggtttcttgttcgtcgtcgataggattaaagaattgatcaagtacaaagccttccaagttgctccagccgagcttgaggagttgttgctttctcacaccgaaatcgccgacagtgccgtcatcccatacccagatgacaagaccggtcaaatcccaatggccttcatcgttagaaagtctggttctaaggtcaaggaggaggacgttatgagtttcgtcgctaagcaagttgccccttacaagaaaattagaagagtcgcctttgtttaa

SEQ ID NO: 50 - protein, 513 aa, Dacrycarpus compactus
MSDLNQKGGYCAATGIYHSLREPLPLPPPYQNLDITTFVFSHHHTTDIALIDAHTGCSLSYAALRRNVKSLTAALHHLGIGKGDVVLVLSSNSIHIPCIYMAIFSVGAILTTANPLNTQSEIQIQVTESNPAIIFAATDLVPKARATNRPVVVIDKEKEEQHGCVATLWKLLQSSVNEARKLDIHQDDTATLLYSSGTTGKSKGVVGSHRNFISMVAGLVSGANEWNSVVMMTMPMFHVYGFLFAMSFLASGSTVVVMPKFDFALMLWSVQRYRVKYLPTSPPLFVALTKSPMVEKYDLSSLDRVVSGGAPLGKEVIDEFANRFPDVEVAQGYGLTESCGAVTFTSSAEEKKKYGTAGLLAANMEAKIVDTVTGKALPPNQRGELWLRGPSIMKGYYNNEEATAATLDSEGWLKTGDLCYIDDEGFLFVVDRIKELIKYKAFQVAPAELEELLLSHTEIADSAVIPYPDDKTGQIPMAFIVRKSGSKVKEEDVMSFVAKQVAPYKKIRRVAFV

SEQ ID NO: 51 - DNA, 1650 bp, Cinnamomum micranthum f. kanehirae
atggagcatttgaagccttctagtgccaacagtagtcctttgacccctatcggtttccttgatagaactgccaccgtctacggtgactgcccttctattatctataacgacaccatctacacttggtctcaaactagaaatagatgcattagattggccagtgccttgtctttgatcggtatcagtagtggtgacgtcgtctctgtcgttgctccaaacatcccagccatgtacgagatgcacttcggaattcctatgtgcggtgctatccttaacaccattaatatcagacttgacagtaggaccatcagtgtccttcttaggcacagtgactctaaattggtttttgtcgatattcaaagtagatctcttattgaacaagctgtcaagtctcttaccaacccacctagattgatcattatcgaggatgaatacgagaacggtggtagagtcgagggtgacaattacggtgagcttacctacgagaggatgatcgagaagggtgaccctaggtttgagtgggttagacctaagtctgaatggtctccaatgattttgaattatacctctggaaccacctcttctcctaagggtgtcgtccacagtcatagaggtatcttgatcatcaccgtcgacagtttgatcgactggatgggtaccaagcagccagtttacctttggaccttgcctatgttccacgccaacggttggagtttcccatggggagttgctgccatgggaggtaccaatgtctgccttaggaagttcgatgctagaatcgtctatggagctatccatagacatagggtcacccacttgtgtggtgccccagttgtcttgaacatgcttgccaatgctccagacagtgatagaaagccacttcctaacccagttcagatccttaccgctggtgctcctccaccagctgctgtcctttttagaaccgaagccttgggattctctgtctctcacggttacggattgaccgaaaccgccggtcttgttgtcagttgcgcttggaaaggtcagtggaacagacttccagctaccgagagggccagattgaaggctaggcaaggagttaggaccttgtgcatgaccgaagtcgatgttgtcgacgagaagaccggtaaaggtgtcaagagggacggattgacccttggtgaaattgtccttaggggtggtggtatccttttgggttatttgaaagacccaaaaggtaccgctaatagtttgaagaacggatggttttacaccggtgacgttggagtcatgcatccagatggttaccttgagattagagatagaagtaaggacgtcatcattagtggtggtgagaacttgagtagtgtcgaggtcgagagtgtcttgtactctcacccagctatcaatgagggagctgttgtcgccagaccagatgaattctggggtgaaaccccatgcgccttcgtctctttgaaggaagagttcaaagagaaaaagcctagtgagaaggagattatcgctttctgtagagagaggttgcctcactacatggtcccaaagaccgttgtcattgtcgccgagttgccaaagaccagtaccggtaaggtccaaaagttcgtcttgagggacatggccaagggattgggtccacttcctaaagcttaa

SEQ ID NO: 52 - protein, 549 aa, Cinnamomum micranthum f. kanehirae
MEHLKPSSANSSPLTPIGFLDRTATVYGDCPSIIYNDTIYTWSQTRNRCIRLASALSLIGISSGDVVSVVAPNIPAMYEMHFGIPMCGAILNTINIRLDSRTISVLLRHSDSKLVFVDIQSRSLIEQAVKSLTNPPRLIIIEDEYENGGRVEGDNYGELTYERMIEKGDPRFEWVRPKSEWSPMILNYTSGTTSSPKGVVHSHRGILIITVDSLIDWMGTKQPVYLWTLPMFHANGWSFPWGVAAMGGTNVCLRKFDARIVYGAIHRHRVTHLCGAPVVLNMLANAPDSDRKPLPNPVQILTAGAPPPAAVLFRTEALGFSVSHGYGLTETAGLVVSCAWKGQWNRLPATERARLKARQGVRTLCMTEVDVVDEKTGKGVKRDGLTLGEIVLRGGGILLGYLKDPKGTANSLKNGWFYTGDVGVMHPDGYLEIRDRSKDVIISGGENLSSVEVESVLYSHPAINEGAVVARPDEFWGETPCAFVSLKEEFKEKKPSEKEIIAFCRERLPHYMVPKTVVIVAELPKTSTGKVQKFVLRDMAKGLGPLPKA

SEQ ID NO: 53 - DNA, 1620 bp, Calocedrus decurrens
atggcttctgatcttaatttgtcttctggtttctctatggccaccggaatctataacagtaagagggacccagttcctttgccacctccacaccaacaccttgaccttaccacctacgtcttctctcacccacacaacaccgagatcgctatcattgacgctcagtctggagagcagctttcttatagagcccttagaaggaatgttagagctttggccactggacttcagaggttgggaattagaaagggtgacgttgtccttgtcatcttgccaaatattatccacgtcccatgcatctacttggccatcgtctctatcggtgccatccttactactgccaaccctcttaacaccgaggctgagatgaggagacaagttgccgattctaacccagttcttgccttcgctgccccagaatttgctcacaaagctagggctgcccagttgccagtcgtccttaccgaaaagacctctccaactgccaaccagtctggatacgttgccacccttcaagaattgttccaatctgacgtcgacgatttccagtctgtcgatatccagcaagatgacaccgccacccttctttacagttctggtaccaccggaaagtctaagggtgtcgtcgccacccataggaatcacatcgccatggtcgctggtttcgtcaataaaatcgatactagaaacagagtcaccttgtgcaccatgcctcttttccatgtctacggttttttctacagtgtcagtagtatcgccaccggaaccaagatgatccttatggccaagttcgacttcgcccaaatgttggcttgtgtcgaaagacatagagtcacttctcttccagtcgcccctcctatcttcgtcgccttgaccaagtctcctatggtcgccaagtacgatttgtcttctttggaaggaatcggtagtggtggtgctgcccttggaaaggagactatcgatgagtttatcaagttgttccctaacgttgaggtcagtcaaggttacggtcttaccgagtctagtggagccgttaccttcacctctaccggagaagagaagaagaagtacggaaccgctggtttgcttgccgccaacgtcgaggccaagatcgtcgatactgtcagtggtaaggcccttcctccaaaccagcaaggagaattgtggcttaggggaccaaccatcatgaagggttacttctctaacgacgaagccaccgctactaccttggactctgagggatggcttaagaccggtgacttgtgctacttcgacgaggaaggtttcttgttcgtcgttgatagaatcaaagaacttattaagtacaagggatatcaagttgccccagctgaacttgaagagcttttgctttctaacccagagatctctgacgccgctgtcattccatacccagacgaagaggccggtcaaatcccaatggcctttgtcgttaggaaggctgactctaagcttaacgaggaggacgttaagagttttgtctctaagcaagttgccccatacaaaaagattaggagggtcgccttcgtcccatctatccctaagtctccatctggtaaaatcttgaggaaggaccttattcagcagaccgtcacctcttaa

SEQ ID NO: 54 - protein, 539 aa, Calocedrus decurrens
MASDLNLSSGFSMATGIYNSKRDPVPLPPPHQHLDLTTYVFSHPHNTEIAIIDAQSGEQLSYRALRRNVRALATGLQRLGIRKGDVVLVILPNIIHVPCIYLAIVSIGAILTTANPLNTEAEMRRQVADSNPVLAFAAPEFAHKARAAQLPVVLTEKTSPTANQSGYVATLQELFQSDVDDFQSVDIQQDDTATLLYSSGTTGKSKGVVATHRNHIAMVAGFVNKIDTRNRVTLCTMPLFHVYGFFYSVSSIATGTKMILMAKFDFAQMLACVERHRVTSLPVAPPIFVALTKSPMVAKYDLSSLEGIGSGGAALGKETIDEFIKLFPNVEVSQGYGLTESSGAVTFTSTGEEKKKYGTAGLLAANVEAKIVDTVSGKALPPNQQGELWLRGPTIMKGYFSNDEATATTLDSEGWLKTGDLCYFDEEGFLFVVDRIKELIKYKGYQVAPAELEELLLSNPEISDAAVIPYPDEEAGQIPMAFVVRKADSKLNEEDVKSFVSKQVAPYKKIRRVAFVPSIPKSPSGKILRKDLIQQTVTS

SEQ ID NO: 55 - DNA, 1602 bp, Podocarpus rubens
atgtctgtctctaaccagaaaagaggatactctgccgccaccggaatttaccacacccttagggaccctttgcctttgcctccaccaacccaaaggttggacatcaccaccttcgtcttctctcaccatgacatggtcttgatcgacgcccctaccggatgttctcttagttacgctgcccttaggaggaacgtcaagtctcttagtgctgcccttcaccacttgggtatcggtaagggtgacgtcgtcttggtcttgtctcctaactctatccatattttgtgcatttatatggctatcttcagtatcggagctatcttgaccaccgccaacccattgaataccgagagtgagatccttaaccaagttaccgagtctaacccagctatcgttttcgctgccccagacttggttcctaaggctagaaccactagaagaccagttgtcatcatcgacaacgaaaagcagaggcagcacggatgtgtcggtaccttgcttgagttgcttcagtactctgtcaacgacgccccttctgtcgacattgagcaagatgacaccgccaccttgttgtattcttctggaaccaccggtaagtctaagggtgtcatcggttctcatagaaatttcatctctatggttgccggtcttttgtctggtgccaacgagtggaacaccgtcgtcatgatgaccatgccaatgttccacgtctacggtttccttttcgccatgtctttccttgcctctggttctaccgtcgtcgttatgccaaagttcgatttcgcccttatgctttggagtgtccagtgctatagagtcacttacttgcctacttctccacctcttttcgtcgcccttaccaagtctccaatggtcgagaagtacgacctttcttctcttaagagagtcgcttctggtggtgctcctttgggaaaagaagttattgatgaatttgtttatagattcccaaaggttgaagttgcccaaggttacggtttgaccgagtcttgtggtgctgtcaccttcaccagtagtgccgaggagaaaaagaagtacggaaccgccggtttgcttgccgccaacatggaggccaagattgttgacaccgtcaccggaaaagccttgccacctaatcagaggggagagttgtggttgaggggtccaagtattatgaaaggatattacaagaatgaggaggccaccgccgccacccttgacagtgaaggttggcttaagaccggtgacctttgctacatcgatgaggagggtttcttgttcgttgtcgataggattaaagaattgattaaatatagggctttccaagttgccccagccgagttggaggagatccttctttctcacccagagattgccgactgtgccgtcatcccatacccagatttccttaccattatgcctcttgtttttgaccttttgagaaaatctggtagtaaacttaaagaagaagacgtcatgagttttgtttctaagcaagtcgccccttacaaaaagattagaagggtcgccttcgtcaactctattccaaagtctcagtctggtaagatccttagaaaagaatttattcaacaaaacctttctacttaa

SEQ ID NO: 56 - protein, 533 aa, Podocarpus rubens
MSVSNQKRGYSAATGIYHTLRDPLPLPPPTQRLDITTFVFSHHDMVLIDAPTGCSLSYAALRRNVKSLSAALHHLGIGKGDVVLVLSPNSIHILCIYMAIFSIGAILTTANPLNTESEILNQVTESNPAIVFAAPDLVPKARTTRRPVVIIDNEKQRQHGCVGTLLELLQYSVNDAPSVDIEQDDTATLLYSSGTTGKSKGVIGSHRNFISMVAGLLSGANEWNTVVMMTMPMFHVYGFLFAMSFLASGSTVVVMPKFDFALMLWSVQCYRVTYLPTSPPLFVALTKSPMVEKYDLSSLKRVASGGAPLGKEVIDEFVYRFPKVEVAQGYGLTESCGAVTFTSSAEEKKKYGTAGLLAANMEAKIVDTVTGKALPPNQRGELWLRGPSIMKGYYKNEEATAATLDSEGWLKTGDLCYIDEEGFLFVVDRIKELIKYRAFQVAPAELEEILLSHPEIADCAVIPYPDFLTIMPLVFDLLRKSGSKLKEEDVMSFVSKQVAPYKKIRRVAFVNSIPKSQSGKILRKEFIQQNLST

SEQ ID NO: 57 - DNA, 1548 bp, Pseudotaxus chienii
atgcagaagatggaggacttgaagagatgcccagctaactaccctccacttacccctattggattcatcgagagggctgctactgtctacggtgactgcaccagtttggtctacaacaccactagatttacttggagtcagactttcaatagatgcttgaagttggccagtgcccttagttctaggaacatctctaggggtgacgtcgtttctgtcgccgccccaaacgttccagctatgtacgagatgcacttcgccgttccaatggctggtgctgttttgaacaccgtcaacatcagattggacgccagaatcatggctgctcagttcacccactgcgagcctaagttcttgttcgtcgactaccagttccttccagtcgtctctgaagccttgtctggaatcggtcacaagccttgtgtcgtcgtcatcgaggagcttgacaacggtagggaaatcgctacctctgccggacttacctacgaaggtcttatcggagagggtgacaccgaattcgagatcagatggccagaggacgaatggcaagctgccgtcttgaactacacctctggaactacctctgccccaaaaggagtcgtccattgccatagaggaatttacaccatggccatggataacttgttgatgtggggtcttagaactagaccagtttacttgtggacccttgccttgttccacgctaatggttggtgcttcccttggagtcttgccgccatgggaggtaccaacatctgccttaggaagttcgacgctaagatcatcttcgacgccttggccgaacacggtgttacccatatgtgcgccgctccagttgtccttagtatgatcgtcaacgcccacccttctgaaaggagaccagttgctggaagggtcgaaatccttaccgccggatctcctccaccagctgccatccttggtaaggtcgaacaaatgggtttcgccgtcacccatggatacggtttgagtgaaaccgccggtcttgtcgtctcttgtgcttggaaggctgaatgggacaaccttccagccgaagagagggccagattgaagtctaggcaaggtgttagaaacgtctctttggccgaagtcgacgtcaaagacccaaccagtatggctagtgtcgctagagacggtgttcagatcggagaggtcatgatgagaggtgcctctgtcatgaagggttacttgaaaaatcaagaaatgaccgctagagccatggatggtggttggtttagaaccggtgatgtcggtgtccttcacccagacggatacttggaaattaaggatagatctaaggacattatcatctctggtggtgaaaacatttcttctgtcgaagttgaatctgtcttgtacagtcacccattgatcgtcgaggccgctgttgtcgctagaccagacccattttggggtgagaccccttgcgccttcgtctctattaacaacaatagtaagcaagccttgagtgaggcccaagttatcagtttttgtagagagaggatgccacacttcatggctccaaaatcttaa

SEQ ID NO: 58 - protein, 515 aa, Pseudotaxus chienii
MQKMEDLKRCPANYPPLTPIGFIERAATVYGDCTSLVYNTTRFTWSQTFNRCLKLASALSSRNISRGDVVSVAAPNVPAMYEMHFAVPMAGAVLNTVNIRLDARIMAAQFTHCEPKFLFVDYQFLPVVSEALSGIGHKPCVVVIEELDNGREIATSAGLTYEGLIGEGDTEFEIRWPEDEWQAAVLNYTSGTTSAPKGVVHCHRGIYTMAMDNLLMWGLRTRPVYLWTLALFHANGWCFPWSLAAMGGTNICLRKFDAKIIFDALAEHGVTHMCAAPVVLSMIVNAHPSERRPVAGRVEILTAGSPPPAAILGKVEQMGFAVTHGYGLSETAGLVVSCAWKAEWDNLPAEERARLKSRQGVRNVSLAEVDVKDPTSMASVARDGVQIGEVMMRGASVMKGYLKNQEMTARAMDGGWFRTGDVGVLHPDGYLEIKDRSKDIIISGGENISSVEVESVLYSHPLIVEAAVVARPDPFWGETPCAFVSINNNSKQALSEAQVISFCRERMPHFMAPKS

SEQ ID NO: 59 - DNA, 1614 bp, Taxus x media
atggctagtgaccttgacccatcttctggtttctgtagagccgctggtatctactactctaaaagggacccaatcgaacttcctcctccagatcagcacttggaccttaccacctacgtcttcagtcaccaccacaataccgagatcgccttcatcaatgccccatctggagcccagttgtcttactctgcccttaggcacaacgtcaaggctttggctgcctctcttcagaggcttggtattagaaagagagacgtcgtcttggtcatctctcctaactctatccatttgccatgtatttaccttgccatcgtctatatcggtgccatcttgaccaccaccaacccacttaacgctgaggctgagattagaaagcaaatcgccgacagtaacccagttttggtcttcgccgccccagaattccttccaaaagctagagctgccagattgccagtcgtccttatccagaccaccaaccaaaccgccatcccaagtggatgcgtcgccactcttcacgagttgttccaatctgacgtcgacgactttccatctgtcgacgtcaagcaagacgacactgccaccttgttgtacagttctggaaccactggtaagagtaagggagtcgtcggaacccataggaatcacatcgccatggttgccggatacgtcaataaaactagaaaatttatcaacttgtgtactatgcctatgttccacgtttatggtttcttctatactttgagtgctgtcgcctctggaagtaccatggtcgtcatgcctaagttcgacttctctgaaatgttggccaccgtcaagaggtacagagttacctctttgccagctgcccctcctcttttcgtcgcccttaccaagtctcctatcgtcgcccagtacgaccttagtagtcttcagagtgtcggttctggtggagcccctttgtctaaggagatcatcgacaaatttatcgccatgttccctaacattgaagtcgcccaaggttacggacttactgagagtaacggagccgtcactttcacctctactagtgaagagaacaagaaatacggtaccgccggtttgcttgctgccaacgtcgaggccaaaatcgtcgacaccgtcagtggaaaagccttgccacctaaccagaggggtgaattgtggttgaggggacctaccgtcatgaagggatatttcggtaatatggaggctaccgccgccacccttgactctgagggttggcttaagaccggagacttgtgttacatcgatgaagaaggatttttgttcgtcgttgacagaatcaaggaattgatcaagtataaggcttaccaagttgccccagccgagttggaggagttgttgttgtctaacccagaaatcgctgacgccgctgtcattccatacccagacaaggaagccggtcagatccctatggcttttatcgtcagaaaaagtgattctaagttgaaagaagaagatgtcatgtctttcgtcagtaaacaagttgccccttacaagaagattagaagggtcagtttcgtcgcctctatcccaaagtctccaaccggaaaaattttgagaaaagatcttatccaacagaccttgagtacctaa

SEQ ID NO: 60 - protein, 537 aa, Taxus x media
MASDLDPSSGFCRAAGIYYSKRDPIELPPPDQHLDLTTYVFSHHHNTEIAFINAPSGAQLSYSALRHNVKALAASLQRLGIRKRDVVLVISPNSIHLPCIYLAIVYIGAILTTTNPLNAEAEIRKQIADSNPVLVFAAPEFLPKARAARLPVVLIQTTNQTAIPSGCVATLHELFQSDVDDFPSVDVKQDDTATLLYSSGTTGKSKGVVGTHRNHIAMVAGYVNKTRKFINLCTMPMFHVYGFFYTLSAVASGSTMVVMPKFDFSEMLATVKRYRVTSLPAAPPLFVALTKSPIVAQYDLSSLQSVGSGGAPLSKEIIDKFIAMFPNIEVAQGYGLTESNGAVTFTSTSEENKKYGTAGLLAANVEAKIVDTVSGKALPPNQRGELWLRGPTVMKGYFGNMEATAATLDSEGWLKTGDLCYIDEEGFLFVVDRIKELIKYKAYQVAPAELEELLLSNPEIADAAVIPYPDKEAGQIPMAFIVRKSDSKLKEEDVMSFVSKQVAPYKKIRRVSFVASIPKSPTGKILRKDLIQQTLST

SEQ ID NO: 61 - DNA, 1608 bp, Tetraclinis sp.
atgaatcatagtagtggtttctctatggctagtggtatttattattctaagagggacccagttcctttgcctcctcctcaccaacaccttgacttgaccacctacgtcttctctcaccctcacaacaccgagatcgctttgactgacgcccagtctggtgagcaattgtcttacaccaccttgaggcacaacgttagagcccttggaaccggtttgcagaggcttggtattagaaagggtgacgtcgtccttgttatccttccaaatattatccacgttccatgcatctaccttgccatcgtttctatcggagccatcttgactaccgccaacccacttaacaccgaggctgagatgagaaggcaagttgccgactctaacccagttttggctttcgctgctccagagttcgctcataaggctcacgccgcccagcttccagttgtcttgaccgagaagaccaaccctaccgccaatcagtctggatatgtcgctaccttgcaagagttgttccagaccgatgtcggtgagttccagtctgttgatgtccaacaagatgacaccgctaccttgttgtacagtagtggaaccaccggtaggagtaaaggagtcgttgccacccataggaaccatatcgctatggttgccggtttcgtcaacaagatcgacacccaaaatagagtcaccctttgcaccatgcctttgttccatgtctacggatttttctattctgtttctagtattgctactggtactaaaatgattttgatggccaaattcgacttcgtccagatgttggcttgcgttgagaggcataaagtcaccaacttgccagtcgctccaccaatcttcgtcgccttgaccaagagtccaatggtcgccaagtacgacttgagtagtcttgagggtattggtagtggtggtgctgccttgggtaaagagaccatcgatgaatttatcaagcttttccctaaggtcgaggtcagtcaaggttatggacttaccgagagttctggtgccgttaccttcaccagtaccggtgaggagaagaagaagtacggtaccgccggtttgttggccgccaatgtcgaggccaaaatcgtcgataccgtctctggaaaggccttgcctcctaatcagagaggagagttgtggcttaggggtcctaccgtcatgaaaggttacttcagtaatgatgaggctaccgtcgccaccttggactctgaaggttggcttaagaccggtgacttgtgctacttcgacgaggagggtttcttgttcgttgtcgatagaattaaagaattgatcaagtacaaaggttaccaagttgccccagctgagttggaagagcttttgcttagtaacccagagatcagtgacgccgccgtcatcccatacccagatgaagaggccggtcaaatccctatggcctttgtcgttagaaagccagacaccaaacttaacgaggaggacgtcaagtctttcgtctctaagcaagttgccccatacaagaagattagaagggtcgctttcgtcccttctatccctaaatctccttctggtaaaattcttaggaaggatttgatccagcagactgtcacctcttaa

SEQ ID NO: 62 - protein, 535 aa, Tetraclinis sp.
MNHSSGFSMASGIYYSKRDPVPLPPPHQHLDLTTYVFSHPHNTEIALTDAQSGEQLSYTTLRHNVRALGTGLQRLGIRKGDVVLVILPNIIHVPCIYLAIVSIGAILTTANPLNTEAEMRRQVADSNPVLAFAAPEFAHKAHAAQLPVVLTEKTNPTANQSGYVATLQELFQTDVGEFQSVDVQQDDTATLLYSSGTTGRSKGVVATHRNHIAMVAGFVNKIDTQNRVTLCTMPLFHVYGFFYSVSSIATGTKMILMAKFDFVQMLACVERHKVTNLPVAPPIFVALTKSPMVAKYDLSSLEGIGSGGAALGKETIDEFIKLFPKVEVSQGYGLTESSGAVTFTSTGEEKKKYGTAGLLAANVEAKIVDTVSGKALPPNQRGELWLRGPTVMKGYFSNDEATVATLDSEGWLKTGDLCYFDEEGFLFVVDRIKELIKYKGYQVAPAELEELLLSNPEISDAAVIPYPDEEAGQIPMAFVVRKPDTKLNEEDVKSFVSKQVAPYKKIRRVAFVPSIPKSPSGKILRKDLIQQTVTS

SEQ ID NO: 63 - DNA, 1515 bp, Nageia nagi
atggatcctatgagtggtttttgcacttctaatggtgtctattacagtaagagggaaccttctgcccttccatctcctcaccaaaacatggacatcaccacctacgccttcagtcaccaccataccaccaacatcgcttttgtcgacgcccctaccggtagatctctttcttattctaccttgagaagaaacgtcaaggcccttgctgccggacttcacagacttggagttcagaaggacgacgtcgttttggtcctttctcctaactctatcgacatcccttgcatctacatgggaattctttctttgggtgccatccttactaccgccaacccattgaacaccgaggccgagatccagaagcaagtcgctgacagtaacccagctattgtcttcgccgctccagagttgttggacaaagctagggccactcagaggccagtcgttatcatcggtgacgaaaaccagacccttccacacggttgcgtcgcctctttgcaagagttgcttcaaagtccaatcgacggtgcccctccagttgagattaagcaagaagataccgccactttgctttactcttctggtaccaccggaaaagctaagggagttgtcgccacccatagaaactacatcgccatgatggccggacttgtcaacacccccggtgatgacgtcgaaaaggacgtctatttgttgattatgcctttgttccacgtctacggtttctttagaatgatctggtctgtcgccatgggtaacaccgtcgtcgtcatgccaaagttcgatttggcccagatgctttggaacatcgagagatatagagtcacttgtatcccagctgcccctcctattttcgtcgcccttgccaagagtcctatcgttgaaaaatatgacttgtctagtcttcagagaatcggtagtggaggagccgctttgggtaaagaagttatcgaagagttcatgggtaggtttcctagggtcgaagtcggtgctgctaccttgaccgttagtgccgaggagaagaagaaatacggtaccgccggacttttgcttgccaacatggaggccaagattgtcaacaccgtcactggaaagccattgcctccaaatagaagaggtgagttgtggcttaggggaccttgcatcatgaagggttactacaacaacaaggaggctaccactgccaccttggactctgagggttggttgaagaccggtgacctttgctacatcgacgaggagggattcttgttcgttgtcgatagaattaaggagttgatcaagtataaggcctttcaagttgctccagccgaattggaggagcttttgcttagtcacccagagatcgccgattgcgctgttatcccatacccagatgacgaagctggacaaatcccaatggccttcgtcgtcatcaagagtggttctaagcttaaagaggaagacgtcatgagtttcattagtaaacaagtcgccccttacaaaaagattagaagggtcgccttcgtcaactaa

SEQ ID NO: 64 - protein, 504 aa, Nageia nagi
MDPMSGFCTSNGVYYSKREPSALPSPHQNMDITTYAFSHHHTTNIAFVDAPTGRSLSYSTLRRNVKALAAGLHRLGVQKDDVVLVLSPNSIDIPCIYMGILSLGAILTTANPLNTEAEIQKQVADSNPAIVFAAPELLDKARATQRPVVIIGDENQTLPHGCVASLQELLQSPIDGAPPVEIKQEDTATLLYSSGTTGKAKGVVATHRNYIAMMAGLVNTPGDDVEKDVYLLIMPLFHVYGFFRMIWSVAMGNTVVVMPKFDLAQMLWNIERYRVTCIPAAPPIFVALAKSPIVEKYDLSSLQRIGSGGAALGKEVIEEFMGRFPRVEVGAATLTVSAEEKKKYGTAGLLLANMEAKIVNTVTGKPLPPNRRGELWLRGPCIMKGYYNNKEATTATLDSEGWLKTGDLCYIDEEGFLFVVDRIKELIKYKAFQVAPAELEELLLSHPEIADCAVIPYPDDEAGQIPMAFVVIKSGSKLKEEDVMSFISKQVAPYKKIRRVAFVN

SEQ ID NO: 65 - DNA, 1605 bp, Oncotheca balansae
atgcttgacccatctagtggtttctgtagaggatctggtatctactacagtaagagggatccagtcgccatccctcctccagatcagcacttggacttgaccacctacgtcttcagtcaccatcacaacacccagattgctttcattgacgccccttctggtgcccaacttagttatagtagtttgaggcacaatgtcaaggcccttgccgccggacttcagaggcttggtatcagaaagagggacgtcgtcttggttatcagtccaaattctatccaccttccttgcatctacatggccatcgtctacattggtgccatccttaccaccaccaaccctttgaacgccgaggctgaaattagaaagcaagtcgccgattctaactctgtccttgtccttgtcgccccagaattcgttccaaaggctagagctgccagtttgccattcgtccttatccagaccaccaaccaaactgctattccagctggttgcgtcgctaccttgcaagagttgttccaaagtgacgtccacgacttcctttctgtcgacgtcaagcaagatgacaccgccactcttttgtacagttctggtaccactggtaagagtaagggagtcgtcggttctcatagaaatcacatcgccatgcttgccggatttgtcaagaaggccgaaaaggccatcaacctttgcaccatgccaatgttccacgtttacggtttcttctacaccatgggagttgtcgccactggttctaccaccgtcgtcatgccaaagttcgacttcagtgagatgttggcttgtgtcgagaggtatagaatctctagtttgccagctgctccacctttgttcgttgcccttaccaagagtccaatcgttgccaactacgacctttcttctcttaaaagaatcggtgctggtggtgccccattgggtaaagagatcatcgacgagcttatcgccgtcttgcctaacatcgaggtcgcccaaggatacggtcttaccgagagtaacggtgccgttaccttcaccagtaccggtgaaggtaacaagaagtacggaaccgccggattgttgagtgccaacatggaggccaaaatcgtcgataccgtcaccggtaaagctttggcccctaaccagaggggtgagcttcttttgagaggtccaaccgttatgaagggttactttggtaacgtcgaagccactaaggccactcttgactctgacggttggcttaagaccggagacttgtgctatatcgacgaggagggattccttttcgtcgtcgatagaattaaggagttgatcaagtacaaggcttaccaagttgccccagctgaacttgaggagcttttgttgtctaacccagaaatcgccgacgctgccgttattccttacccagacaaagaggccggacagatcccaatggccttcatcgttagaaagagtgattctaaattgaaagaggaagatgttatgtctttcgtcagtaagcaagttgccccatacaagaaaattaggagggtcgccttcgttacctctatccctaaaagtcctaccggtaagatccttaggaaggaccttattcaacagaccttgtctacttaa

SEQ ID NO: 66 - protein, 534 aa, Oncotheca balansae
MLDPSSGFCRGSGIYYSKRDPVAIPPPDQHLDLTTYVFSHHHNTQIAFIDAPSGAQLSYSSLRHNVKALAAGLQRLGIRKRDVVLVISPNSIHLPCIYMAIVYIGAILTTTNPLNAEAEIRKQVADSNSVLVLVAPEFVPKARAASLPFVLIQTTNQTAIPAGCVATLQELFQSDVHDFLSVDVKQDDTATLLYSSGTTGKSKGVVGSHRNHIAMLAGFVKKAEKAINLCTMPMFHVYGFFYTMGVVATGSTTVVMPKFDFSEMLACVERYRISSLPAAPPLFVALTKSPIVANYDLSSLKRIGAGGAPLGKEIIDELIAVLPNIEVAQGYGLTESNGAVTFTSTGEGNKKYGTAGLLSANMEAKIVDTVTGKALAPNQRGELLLRGPTVMKGYFGNVEATKATLDSDGWLKTGDLCYIDEEGFLFVVDRIKELIKYKAYQVAPAELEELLLSNPEIADAAVIPYPDKEAGQIPMAFIVRKSDSKLKEEDVMSFVSKQVAPYKKIRRVAFVTSIPKSPTGKILRKDLIQQTLST

SEQ ID NO: 67 - DNA, 1590 bp, Glyptostrobus pensilis
atgccatcttctggttactctagggccaccggaacctactacagtaagagagacccagtccctcttcctcctcctcaccagcacttggacttgaccacctacgttttcagtcacaagcacaataccgagaccgccttcatcgatgctcaatctggagcccagttgtcttatagagccttgagggataacgttagagcccttgccaccggtttgcagaggttgggaatcagaaagagggacgtcgttcttgttattttgccaaataatattcacgttccatgtatctacttggctatcgttagtatcggtgccatcgtcaccaccgctaacccattgaacaccgaagccgagcttagaaggcaagttgccgattctaatccacttcttgctttcgccgctccagaattcgctcataaagccagagccgctcaattgccagtcgtcttgacccagaagacctctccaatcgctaacgactctggatacgttgctaccttgcacgagttgttcgaaagtgacgtcgacgacttccagagtgtcgacatccaacaagaagacatcgctactttgttgtattctagtggtaccaccggaaagaacaagggagtcatcagtacccatagaaaccaaatcgccatggttgccggaatcttgcagaggaccgagccagaaaagaacatcgtcctttgcatcgtcccacttttccacgtctacggtttcttctatagtatctgcaccgtcgctaggggaaccactttggtccttatggccaagtttgacttcgcccagatgttggccaacgttgagaggtatagaatcatcacccttgctattgccccaccaatcttcgtcgcccttaccaagagtccaatcgtcgccaagtacgacttgtcttctttgaaaagaatcggttctggtggtgcccctcttggaaaggagactatcgacgaacttatgacccttttccctaacatcgaggtctctcaaggttacggattgaccgagagttctggtgccgttaccttcacttctaccggagaggaaaagaaaaaatacggtactgccggtttgttggccgccaacgtcgaggctaaaattgtcgatattgtcagtggaaaggccttgccacctaaccagcaaggagaattgtggttgaggggtcctaccatcatgaagggttacttttctaacgacgaagccaccgccaccaccttggactctgagggttggttgaagaccggagacttgtgctacttcgacgaagaaggttttttgttcgttgttgatagaattaaagagttgatcaagtacaaaggttaccaagttgccccagctgaacttgaagagttgcttcttagtaacccagaaatctctgacgccgccgttattccttacccagataaggaggccggtcagatccctatggccttcatcgttagaaaggccgactctaagttgaacgaggaggatgtcaagagttttgtctctaagcaagttgccccatataagaagattagaagggtcgccttcgttacctctatccctaagagtgcctctggaaagatccttagaaaggatttgattcaataa

SEQ ID NO: 68 - protein, 529 aa, Glyptostrobus pensilis
MPSSGYSRATGTYYSKRDPVPLPPPHQHLDLTTYVFSHKHNTETAFIDAQSGAQLSYRALRDNVRALATGLQRLGIRKRDVVLVILPNNIHVPCIYLAIVSIGAIVTTANPLNTEAELRRQVADSNPLLAFAAPEFAHKARAAQLPVVLTQKTSPIANDSGYVATLHELFESDVDDFQSVDIQQEDIATLLYSSGTTGKNKGVISTHRNQIAMVAGILQRTEPEKNIVLCIVPLFHVYGFFYSICTVARGTTLVLMAKFDFAQMLANVERYRIITLAIAPPIFVALTKSPIVAKYDLSSLKRIGSGGAPLGKETIDELMTLFPNIEVSQGYGLTESSGAVTFTSTGEEKKKYGTAGLLAANVEAKIVDIVSGKALPPNQQGELWLRGPTIMKGYFSNDEATATTLDSEGWLKTGDLCYFDEEGFLFVVDRIKELIKYKGYQVAPAELEELLLSNPEISDAAVIPYPDKEAGQIPMAFIVRKADSKLNEEDVKSFVSKQVAPYKKIRRVAFVTSIPKSASGKILRKDLIQ

SEQ ID NO: 69 - DNA, 1620 bp, Picea engelmannii
atgattgatcctaaaagtggatattgtagagaaaatggtatttactatagtaagagagctcctatcgacttgcctccaccaacccaacaccttgatgtcaccacctacatcttctctcaccaccatcacacccagcaaatcgccttcatcgacgcctcttctggattgagtttgtcttacccagctttgaggcagaacgttagagctttggccgccggtatgcatggacttggtattagaaagggagacgttgtcttggtcatctctcctaacagtatcgcccttccttgcatctacttggccatcgtctctatcggagccatccttactaccgccaaccctatcagtaccgaggccgagatcaagaagcaagctgaagacagtaagccagttattgttttcaccccagccaaccttattaacaaagttagagccacccagcttccattcattttgatcgaaggtaacaccgaagaggtcagtggttctggatgcatcagttctcttaaccttttgttgaggtctgccatcaccggattcccaagtgtcgacatcaagcaagaagacaccgctaccttgttgtactctagtggtaccaccggtaaaagtaagggagtcatctctacccatagaaacttgatcgccatgatcgccggtgtccttaataccgacgatgacatggagggtgtcgagaagatctgtttgtgcatcatcccattgttccacgtcttcggtttcttctacaccgtcagttgcattgccaccggtaacaccatggtcgtcatgccaaagttcgaccttaccgagatgttgagtgctatccagcagtttagagttaagtctcttccagcttctccaccaatcttggtcgcccttaacaagagtccagtcgttgccaagtacgatttgacctctctttactctatcgcttgtggtggagctccacttggtaaggacgtcatcgacaacttcaccgctaggtttcctaccgtccaagtccaacaaggttacggtttgaccgagtcttctggaagtgtcgccttcagtaagaccgacgaggagaacaagcattacggtaccgctggtcttttggccggtaacgttgaagctaaggtcgttgacaccgctaacggtaatgccatgccacctaaccacaagggtgaattgtggttgaggggtccaaccatcatgaagggatacttcggaaatgatgaagccaccgccagtactttggacaccgagggttggttgaagaccggagatttgtgctacatcgacgaagagggtttcttgtttgtcgtcgatagaattaaggaattgattaaatataaggccttccaagtcgccccagccgagttggaagagttgttgttgagtaactctgagatctctgacgccgtcatcccatacccagatgacgaagctggtcagatccctatggccttcgtcgttaggaggagtgattctaacctttctaaagaggacgtcatcaacttcgccgctaagcaagttagtccttacaagaagattagaagggttgccttcgtcaactctatcccaaaaagtccaagtggtaagatcttgagaaaagacttgattcatcaagccttgagtacttaa

SEQ ID NO: 70 - protein, 539 aa, Picea engelmannii
MIDPKSGYCRENGIYYSKRAPIDLPPPTQHLDVTTYIFSHHHHTQQIAFIDASSGLSLSYPALRQNVRALAAGMHGLGIRKGDVVLVISPNSIALPCIYLAIVSIGAILTTANPISTEAEIKKQAEDSKPVIVFTPANLINKVRATQLPFILIEGNTEEVSGSGCISSLNLLLRSAITGFPSVDIKQEDTATLLYSSGTTGKSKGVISTHRNLIAMIAGVLNTDDDMEGVEKICLCIIPLFHVFGFFYTVSCIATGNTMVVMPKFDLTEMLSAIQQFRVKSLPASPPILVALNKSPVVAKYDLTSLYSIACGGAPLGKDVIDNFTARFPTVQVQQGYGLTESSGSVAFSKTDEENKHYGTAGLLAGNVEAKVVDTANGNAMPPNHKGELWLRGPTIMKGYFGNDEATASTLDTEGWLKTGDLCYIDEEGFLFVVDRIKELIKYKAFQVAPAELEELLLSNSEISDAVIPYPDDEAGQIPMAFVVRRSDSNLSKEDVINFAAKQVSPYKKIRRVAFVNSIPKSPSGKILRKDLIHQALST

SEQ ID NO: 71 - DNA, 1650 bp, Cupressus dupreziana
atggagcagatggaggaattgaagaggtgcccagctaactaccctccttttaccccaatcggtttcgtcgagagggccgctatcgtctactctgattgcacctctgtcgtctacaacaccactagatttacttggtctcagaccttcaaaaggtgtagacagttggcctctggattggcccttaggaacatctgtagaggtgacgtcgtctctgtcgtcgccccaaatatcccagccatgtatgagatgcacttcgctgttcctatggccggtgccgtcttgaacaacttgaacactagattggacgccagaactatggctgcccagttgagtcattgcgagccaaagatcatcatcgccgactaccagttcttgtcttctgtcaacgaaaccctttctttgcttaagcacaagccaatccttgtcgtcatcgaggagatggaatctggaaaggagatcggtaacggttctgtccttacctacgagggtttgttgagggagggagacccagaattcgagattagatggccagaagatgagtggcaagctgccgttcttaactacacctctggtaccacctctgccccaaaaggtgtcgttcagagtcatagaggaatctacgctatggcccttgacaacttgaccatgtggcagatgggaaggaggccagtttacttgtggactttggccatgttccatgctaacggttggtgccttccttggacccttgccgccgtcggtggtatcaatatttgccttagaaagttcgatgctaaaactatctttgactctatcgccgagcacaacgtcacccatatgtgcggtgccccagtcgttttgtctatgatggccaacgccgatccagctgatagaaagaagttgtctcatagagtcgagatccttaccgctggtgctccaccaccagctgctatcttgtggaagatggaggagttgggattcagtatcacccacggatacggtttgaccgagaccgctggtttggtcgtcagttgtgcttggaaaaccgaatgggacggattgcccggtaaagagaaggctaggcttaagagtaggcaaggtgttagaaacttgagtttggccgaggtcgacgtcaagaatccagttaccatggctgctgtcgccagagatggagtccagatgggagaggtcatgatgaggggagccagtatcatgaagggttacttgaagaacgagggtatgaccgcctctgccatggaaggaggatggttcagaaccggagatgttgccgtcgttcacccagatggatacatcgagatcaaggatagatctaaagatgtcattattagtggaggtgagaacatctcttctgtcgaggtcgagtctgtcctttactctcacccatctgtcgtcgaggctgctgttgtcgccagaccagatcctttctggggagagaccccatgtgctttcgttagtgtcaagaaacactctggtgagggaaacatggaggtcttgagtgaggccgagatcatcgaattctgtagaaagcatcttgcccacttcatggctccaaagtctgtcgtcttcttgccagagttgcctaagacctctaccggtaagatccagaagttcgtccttagggagatggccaagaaccttcctaactcttaa

SEQ ID NO: 72 - protein, 549 aa, Cupressus dupreziana
MEQMEELKRCPANYPPFTPIGFVERAAIVYSDCTSVVYNTTRFTWSQTFKRCRQLASGLALRNICRGDVVSVVAPNIPAMYEMHFAVPMAGAVLNNLNTRLDARTMAAQLSHCEPKIIIADYQFLSSVNETLSLLKHKPILVVIEEMESGKEIGNGSVLTYEGLLREGDPEFEIRWPEDEWQAAVLNYTSGTTSAPKGVVQSHRGIYAMALDNLTMWQMGRRPVYLWTLAMFHANGWCLPWTLAAVGGINICLRKFDAKTIFDSIAEHNVTHMCGAPVVLSMMANADPADRKKLSHRVEILTAGAPPPAAILWKMEELGFSITHGYGLTETAGLVVSCAWKTEWDGLPGKEKARLKSRQGVRNLSLAEVDVKNPVTMAAVARDGVQMGEVMMRGASIMKGYLKNEGMTASAMEGGWFRTGDVAVVHPDGYIEIKDRSKDVIISGGENISSVEVESVLYSHPSVVEAAVVARPDPFWGETPCAFVSVKKHSGEGNMEVLSEAEIIEFCRKHLAHFMAPKSVVFLPELPKTSTGKIQKFVLREMAKNLPNS

SEQ ID NO: 73 - DNA, 1221 bp, Amentotaxus argotaenia
atgttggatccatcttctggtttctgcagtgccaccggaatcttctacagtaagagagacccagttgccttgccaccaccagacgagcacttggacttgactacctacgtcttctctcaccaccacaacaccgagatggctttcatcgatgccttgagtggtgcccagcttagttactcttcttttagacataacgtcaaggccgttgccgctggtttgcagaggcttggtattagaaagcacgacgtcgtcttggtcatctctccaaatagtattcacttgccatgcatctacttggccatcgtcagtattggtgccattctttctaccgccaacccattgaacgccgaggctgagatcagaaagcaagttgctgactctaacccagttttggcctttgctgctccagaatctcttccaaaggcccatgctgctcaattgccagtcgtcttgatcgagaccaccaaccaagccaccatccctaccggttgcgtcggtaccttgagggaattgttccagtctgacgtccacgacttccagtctgttgacgtcaagcaagatgacaccgccacccttatgtactcttctggtaccaccggtaagagtaagggtgtcgtttctacccatagaaaccatatcgccatggtcgccgtttacgtcaacaagaccgccaaaaatattaacctttgtaccatgccactttttcatgtttatggtttcttctacatgatgagtgccgtcgccaccggttctacccttgtcgtcatgcctaagttcgacttcggagagatgcttgcctctgtccaaaggtacagagtcacctctcttccagctgctcctcctttgttcgtcgcccttaccaagagtccaatcgttgccaaatacgacttgagtagtttgcagaggatcggttctggaggtgctccattggccaaggagatcatcgacgagttgatcgccatgttcccaaacgtcgaggttgcccaaggttacggtttgaccgagtcttctggtggagtcaccttcacctctacttctgagggtaacaagaaatacggtaccgccggtcttttggccgccaacgttgaagccaagatcgtcgacaccgtttctggtaaggccttgcctccaaaccagaggggtaaactttggttgaggggtcctatcgttatgaagggatacttcggaaacactgaggccactgctgccacctttgactaa

SEQ ID NO: 74 - protein, 406 aa, Amentotaxus argotaenia
MLDPSSGFCSATGIFYSKRDPVALPPPDEHLDLTTYVFSHHHNTEMAFIDALSGAQLSYSSFRHNVKAVAAGLQRLGIRKHDVVLVISPNSIHLPCIYLAIVSIGAILSTANPLNAEAEIRKQVADSNPVLAFAAPESLPKAHAAQLPVVLIETTNQATIPTGCVGTLRELFQSDVHDFQSVDVKQDDTATLMYSSGTTGKSKGVVSTHRNHIAMVAVYVNKTAKNINLCTMPLFHVYGFFYMMSAVATGSTLVVMPKFDFGEMLASVQRYRVTSLPAAPPLFVALTKSPIVAKYDLSSLQRIGSGGAPLAKEIIDELIAMFPNVEVAQGYGLTESSGGVTFTSTSEGNKKYGTAGLLAANVEAKIVDTVSGKALPPNQRGKLWLRGPIVMKGYFGNTEATAATFD

SEQ ID NO: 75 - DNA, 1614 bp, Prumnopitys andina
atgagtagtcttaatagatctagaggtggttactgcgcttctaccggtatctaccactctcagagggatccattgccattgccacctccacaccaaagtttggacttgaccacctacgtcttttctcaccatcataccacccacaccgctttgatcgatgccccaactggaaggtctttgtcttacgccgccttgaggagaaacgtcaagtctttggccgctggtttgcacagattcggaattagaaaaggagatgtcgtcttggtcctttctcctaacagtatccatattccttctatctaccttgccatcttgagtttgggtgccatccttaccaccaccaacccacttaacaccgaaagtgagatccttaaacagatcagtggttctaacccagccatcgtcttcgccgccccagctttcgtcttgaaagctagggctactagaagaccagtcgtcgtcatcgacaatgagaaggaggagcaagaaggttgcgtcgccactcttttcgagcttcttcaaagttctgctgacaacgccccttctgttgatgtcagacaagatgacatcgccacccttctttactctagtggtaccaccggaaaatctaagggtgtcatgggatctcataggaactacaccgccgctgtcgagatgcttgtctctaggcctaatgagggttcttctgtcgtcttgatgaccatgccaatgttccatgtctatggtttcttgtttatcgtttcttacttcgccaagggtagtaccttggtcgtcatgcctaaattcgattttgagttgatgttgtcttctgtccagaggtatagagtcggatacttgccaacctctccaccagtcttcgttgccttgaccaagtctcctcttgtccaaaagtacgacttgagttctttggagttggtcggaagtggaggtgccccattgggaaaagaagttatcgacaagttcacttgtagattcccaaacatccaagttggtcaaggttatggtttgaccgagtcttgcggtgccgttacctttgctagtagtgccgaggaaaagaaaaagtatggttctgctggattgcttgccgctaacatggacgccaagatcgttgacaccgtcactggaaaggcccttcctccaaaccaaaggggtgagttgtggcttaggggtccatgcatcatgaggggttactacaacaatgaggaagccaccgctgctacccttgactctgagggatggttgaagaccggagatttgtgctacattgacgaggagggattccttttcgtcgtcgatagaatcaaggaattgattaaatacaaggcttaccaagttgccccagctgagcttgaagagttgcttctttctcacccagaaatcgctgactgcgctgtcatcccatatccagatgatgaagccggtcaaatccctatggcctttatcgttagaaaaagtggttctaagttgaaagaagaggatgttatgagttttgttgccaagcaagttgccccatacaagaagattagaagggtcgccttcgtcaactctatcccaaaaagtcaacaaggtaaaattcttaggaaggatcttatccaacaaactcatagtatctaa

SEQ ID NO: 76 - protein, 537 aa, Prumnopitys andina
MSSLNRSRGGYCASTGIYHSQRDPLPLPPPHQSLDLTTYVFSHHHTTHTALIDAPTGRSLSYAALRRNVKSLAAGLHRFGIRKGDVVLVLSPNSIHIPSIYLAILSLGAILTTTNPLNTESEILKQISGSNPAIVFAAPAFVLKARATRRPVVVIDNEKEEQEGCVATLFELLQSSADNAPSVDVRQDDIATLLYSSGTTGKSKGVMGSHRNYTAAVEMLVSRPNEGSSVVLMTMPMFHVYGFLFIVSYFAKGSTLVVMPKFDFELMLSSVQRYRVGYLPTSPPVFVALTKSPLVQKYDLSSLELVGSGGAPLGKEVIDKFTCRFPNIQVGQGYGLTESCGAVTFASSAEEKKKYGSAGLLAANMDAKIVDTVTGKALPPNQRGELWLRGPCIMRGYYNNEEATAATLDSEGWLKTGDLCYIDEEGFLFVVDRIKELIKYKAYQVAPAELEELLLSHPEIADCAVIPYPDDEAGQIPMAFIVRKSGSKLKEEDVMSFVAKQVAPYKKIRRVAFVNSIPKSQQGKILRKDLIQQTHSI

SEQ ID NO: 77 - DNA, 1638 bp, Abies lasiocarpa
atggttatggacgccaagtctggttactgtagagacaatggtatttactattctaaaagggacccaatcgaccttccacctccaacccagaggcttgacgttaccacctacatcttcagtcaccaccaccacacccagcaaatcgccttgatcgatgcctcttctggtctttctttgtcttacccagatttgcagcacaatgttagagccttggctgccggtcttcatggacttggtattagaaagggagacgtcgtccttgtcatttctagtaactctatcgccgtcccatgcatctacttggccatccttagtatcggagccatcctttctaccgccaacccattgagtactgaggccgagatcaagaagcaagctgaagactctaagccagttatcatctttaccgccgccaatttgattgagaaggctagagccacccaacttccagtcatcttgatcgagggaaacaccgaggagagtagtggaagtgacagtgacagtggttgtattagtactttgaatcttttgttgcagtctgccatcgatggtcttccttctgtcgagatcaagcaagaagacactgccaccttgctttacagttctggtaccaccggtaagagtaagggtgttattagtacccatagaaacatcatctctatgatcgccggtaccttgtcttctagagacgctatggagcaaggtgagaagacctacttgtgcatcatccctctttttcacgtttttggattcttttacaccctttcttgcatcgcctctgcctctaccatggttatcatgccaaaattcgacttggcccagatgttgagtgccatccagcagtatagagttaacagtttgccagctagtcctcctcttcttgttgctcttaataagtctcctatcgtcgctaaatatgatttgtcttctcttcacagtatcgcttgtggtggtgccccattgggtaaggatgtcatcgacaactttaccgctagattccctaacgtccaagttcagcaaggttacggattgaccgagagttctggtagtgtcttcgtcaccatcaccgacgaggagaaaaggcattacggaaccgccggacttcttgccgttaacatggaggctaaggtcgtcgacaccaagaccggtaaggccatgcctcctaaccataagggtgagttgtggttgaggggtcctaccatcatgaagggatacttcggtaacgacgaggccaccgccagtacccttgactctgagggatggcttaagaccggtgatttgtgctatatcgatgatgaaggattcttgttcgttgtcgatagaattaaagagttgatcaaatacaaggccttccaagttgccccagccgagcttgaggagttgttgctttctcacccagagatcactgacgccgctgtcatcccatatccagataacgaggccggtcagattcctatggccttcgtcgtcagaaaaccagatagtaatttgtgtgaggaggatgtcatgaacttcgtcgccaaacaagtttctccttacaagaagattagaagggtcgccttcgtcaacagtattccaaagagtccatctggtaaaatcttgagaaaagatttgatctatcaagctctttctacttaa

SEQ ID NO: 78 - protein, 545 aa, Abies lasiocarpa
MVMDAKSGYCRDNGIYYSKRDPIDLPPPTQRLDVTTYIFSHHHHTQQIALIDASSGLSLSYPDLQHNVRALAAGLHGLGIRKGDVVLVISSNSIAVPCIYLAILSIGAILSTANPLSTEAEIKKQAEDSKPVIIFTAANLIEKARATQLPVILIEGNTEESSGSDSDSGCISTLNLLLQSAIDGLPSVEIKQEDTATLLYSSGTTGKSKGVISTHRNIISMIAGTLSSRDAMEQGEKTYLCIIPLFHVFGFFYTLSCIASASTMVIMPKFDLAQMLSAIQQYRVNSLPASPPLLVALNKSPIVAKYDLSSLHSIACGGAPLGKDVIDNFTARFPNVQVQQGYGLTESSGSVFVTITDEEKRHYGTAGLLAVNMEAKVVDTKTGKAMPPNHKGELWLRGPTIMKGYFGNDEATASTLDSEGWLKTGDLCYIDDEGFLFVVDRIKELIKYKAFQVAPAELEELLLSHPEITDAAVIPYPDNEAGQIPMAFVVRKPDSNLCEEDVMNFVAKQVSPYKKIRRVAFVNSIPKSPSGKILRKDLIYQALST

SEQ ID NO: 79 - DNA, 1629 bp, Cephalotaxus harringtonia
atggaaaacatggaagaacttaagaggtgcgccgctaactaccctcctttgaccccaatcggtttcattgaaagagccgccaccgtctacggagactgtacctctgtcgtctacaacaccactagattcacttggtctcataccttcttgaggtgtagaaagttggcctctgcccttagtagtaggtctatcagtagaggtgacgtcgtctctgtcgtcgctccaaacatcccagctatgtatgagatgcacttcgccgtcccaatggctggagccgtcttgaataacgtcaacattagacttgacgctaggaccatggccgctcagttgaaccactgcaagcctaagttcgtcttcgtcgactaccagttcatgccattggttagagaagcccttactgacttggaacataggcctagtgtcgtcgtcatcgaggagatggacaacggtagagaaatcgctgcctctgccgctttgacctatgaggagttgatcaccgagggtaacccagagttcgagattaggtggccagaggatgagtgggaggctgccgtcttgaactatacctctggaaccacttctgctcctaaaggtgtcgtccactgtcatagaggtatttacgccatggccatggacaaccttcttatgtggggaatgagaactcagccagtcatcttgtggaccttgcctatgttccacgccaatggttggtctgtcccttggagtgtcgctgccatgggtggaactaacatctgcgttagaaagttcgacgccaagatcgtcttcgatgcccttgccgagcataaggttacccacatgtgtggagccccagttgtcttgtctatgatggccaatgcccagccatctgagagaaagcctttgcccggtaaggtcgagatccttaccgctggtgctccaccaccagctgccatcttgtggaagatggaagaaatgggtttctctgtcactcacggttacggtcttactgagactgccggtttggttgtctcttgtgcttggaaggccgagtgggataagcttcccggtaaggaaagggctaggcttaaggccagacaaggtgttagaaacgtcacccttgccgaagtcgacgtcaaggatccagctactatggcttctgtcgctagagacggaagacagatgggagaggtcatgttgaggggagcctctgtcatgaagggttacttgaagaatgagcagatgactgctagggccatggatggtggttggttcagaactggagatgtcggtgtcatccacccagatggttaccttgaaatcaaggacagatctaaggatattattatttctggaggtgagaacatcagttctgtcgaggtcgagtctgtcttgtacagtcacccaatgatccttgaagccgccgttgttgctagaccagacccattctggggagagaccccatgcgctttcgtctctatcaagaataaatctaatgaagttttgagtgaggcccaagttatctctttctgtagagaaaggatggcccacttcatggctcctaagtctgttattattatgcaagaattgccaaaaacttctaccggaaagattcaaaagttcgtccttagagagatggctaagagtctttaa

SEQ ID NO: 80 - protein, 542 aa, Cephalotaxus harringtonia
MENMEELKRCAANYPPLTPIGFIERAATVYGDCTSVVYNTTRFTWSHTFLRCRKLASALSSRSISRGDVVSVVAPNIPAMYEMHFAVPMAGAVLNNVNIRLDARTMAAQLNHCKPKFVFVDYQFMPLVREALTDLEHRPSVVVIEEMDNGREIAASAALTYEELITEGNPEFEIRWPEDEWEAAVLNYTSGTTSAPKGVVHCHRGIYAMAMDNLLMWGMRTQPVILWTLPMFHANGWSVPWSVAAMGGTNICVRKFDAKIVFDALAEHKVTHMCGAPVVLSMMANAQPSERKPLPGKVEILTAGAPPPAAILWKMEEMGFSVTHGYGLTETAGLVVSCAWKAEWDKLPGKERARLKARQGVRNVTLAEVDVKDPATMASVARDGRQMGEVMLRGASVMKGYLKNEQMTARAMDGGWFRTGDVGVIHPDGYLEIKDRSKDIIISGGENISSVEVESVLYSHPMILEAAVVARPDPFWGETPCAFVSIKNKSNEVLSEAQVISFCRERMAHFMAPKSVIIMQELPKTSTGKIQKFVLREMAKSL

SEQ ID NO: 81 - DNA, 1635 bp, Chamaecyparis lawsoniana
atggagcagatggaagagcttaagaggtgcccagctaactacccacctttcaccccagttggattcgttgagagggctgctaccgtttacagtgactgtaccagtatcgtctacaacaccactagattcagttggtctcagactttcaaaagatgtagacagttggcttctgcccttgccttgaggaacatctgtcttggagacgtcgtctctgtcgtcgctcctaacatcccagctatgtacgagatgcacttcgccgtcccaatggccggtgctgtccttaacaaccttaacactagattggacgctaggactatggccgctcagatctctcattgtgagcctaaaattattttcgccgactaccagttcttgtctgtcgtcaacgagaccttgtctttgttgaaaagaaagcctcttttggtcgttatcgaggagatggagaacggaaaggagatcggttctggaccagctttgacctacgaaggtcttcttagggagggtgatccagaattcgagatcctttggccagaagacgaatggcaagccgccgttcttaactacaccagtggtaccaccagtgctccaaagggagttgttcagagtcatagaggtatctacgccatggccttggacaaccttaccatgtggcagatgggtaggagaccagtctacctttggactttggccatgttccacgctaacggttggtctttgccatggactttggctgctgttggaggaaccaacatctgccttagaagatttgacgccaagattatctttgatagtatcgctgaacacaaagttacccacatgtgcggagctccagttgtcctttctatgatggccaacgccgatccagctgatagaaagcaagtttcttaccaagttgagattcttactgccggtgcccctccaccagctgccattttgtggaagatcgaggaacttggattctctatcacccacggttacggtcttaccgagaccgccggtcttgtcgttagttgtgcttggaagaccgagtgggatggattgcccggtaaggagaaggctaggcttaagtctaggcaaggtgtcagaaacttgagtcttgccgaggtcgacgtcaagaacccagtcaccatggcttctgtcgctagagatggagttcagatgggtgagatcatgatgaggggtgcctctgtcatgaagggttatcttaaaaacaaggatatcaccgccagtgccatggaaggtggatggtttaggaccggagatgttgccgtcgtccacccagatggatacatcgagatcaaagatagatctaaggacgtcatcatctctggaggagaaaacatctcttctgtcgaggtcgactctgtcctttactctcaccctagtgtcgttgaggctgctgttgtcgccagaccagaccctttctggggtgaaactccatgcgccttcgtctctgtcaagaagcacggtggagaaggaaacggagaggtcttgagtgaagccggtattatcgagttttgcagaaaacatttggcccacttcatggccccaaagagtgtcatcttcatgcaagaattgccaaagacctctaccggaaaaattcaaaagttcgtcttgagagaaatggctaaataa

SEQ ID NO: 82 - protein, 544 aa, Chamaecyparis lawsoniana
MEQMEELKRCPANYPPFTPVGFVERAATVYSDCTSIVYNTTRFSWSQTFKRCRQLASALALRNICLGDVVSVVAPNIPAMYEMHFAVPMAGAVLNNLNTRLDARTMAAQISHCEPKIIFADYQFLSVVNETLSLLKRKPLLVVIEEMENGKEIGSGPALTYEGLLREGDPEFEILWPEDEWQAAVLNYTSGTTSAPKGVVQSHRGIYAMALDNLTMWQMGRRPVYLWTLAMFHANGWSLPWTLAAVGGTNICLRRFDAKIIFDSIAEHKVTHMCGAPVVLSMMANADPADRKQVSYQVEILTAGAPPPAAILWKIEELGFSITHGYGLTETAGLVVSCAWKTEWDGLPGKEKARLKSRQGVRNLSLAEVDVKNPVTMASVARDGVQMGEIMMRGASVMKGYLKNKDITASAMEGGWFRTGDVAVVHPDGYIEIKDRSKDVIISGGENISSVEVDSVLYSHPSVVEAAVVARPDPFWGETPCAFVSVKKHGGEGNGEVLSEAGIIEFCRKHLAHFMAPKSVIFMQELPKTSTGKIQKFVLREMAK

SEQ ID NO: 83 - DNA, 1266 bp, Cunninghamia lanceolata
atgaacccacttaataccgaggccgagattagaaggcaagtcgccgactctaatccagttttggccttcgccgccccagaattcgctcacaaagccagagccgcccagttgccagttatcttgacccaaaccacttctccaaccgccaaccagagtggttacgtcgccacccttcaagaacttttccagagtgacgtcgacgacttccaatctgtcgacgttcagcaagaggacactgccaccttgctttactctagtggaaccaccggtaagagtaagggtgttatcagtacccatagaaaccacatcgccttgatggccggattcatccagagaagagtctctgagaaaaatattaccttgtgcaccatgccacttttccacgtctacggtttcttctacagtgtcggaaccatcgccaccggtaccaccatgatccttatgtctaagttcgacttcgcccagatgttggccaacgtcgaaaggtatagggtcacctctcttccagtcgctcctccaatcttcgtcgctttgaccaagtctccaatcgtcgctaagtacgatttgtctagtttgcagaggatcggttctggtggtgctgcccttggaaaggaaaccatcgacgagttcatcgctcttttcccaaatattgaagtctctcaaggttacggacttaccgagtcttctggtgctgtcaccttcacctctaccggtgaggaaaggaagaagtacggaaccgccggattgttggccgccaacatggaggccaagattgtcgacatcatctctggtaaggcccttcctcctaatcagaggggtgagttgtggcttaggggaccaaccatcatgaagggatacttttgcaacgccgaagccactgccaccaccttggacagtgagggatggttgaagactggagacttgtgctacttcgacgaggagggattcttgttcgtcgttgatagaattaaggaacttatcaagtacaaaggataccaagttgccccagctgaacttgaggaattgcttttgagtaacccagaaatcgctgacgccgctgtcatcccatacccagataaggaagctggtcaagttccaatggcctttatcgttaggaaggccgacagtaagttgaaggaagaggatgttaaaagttttgttagtaagcaagttgccccttacaagaagattaggagggtcgccttcgtcacctctatccctaagagtgccagtggtaagatcttgagaaaagatttgatccaataa

SEQ ID NO: 84 - protein, 421 aa, Cunninghamia lanceolata
MNPLNTEAEIRRQVADSNPVLAFAAPEFAHKARAAQLPVILTQTTSPTANQSGYVATLQELFQSDVDDFQSVDVQQEDTATLLYSSGTTGKSKGVISTHRNHIALMAGFIQRRVSEKNITLCTMPLFHVYGFFYSVGTIATGTTMILMSKFDFAQMLANVERYRVTSLPVAPPIFVALTKSPIVAKYDLSSLQRIGSGGAALGKETIDEFIALFPNIEVSQGYGLTESSGAVTFTSTGEERKKYGTAGLLAANMEAKIVDIISGKALPPNQRGELWLRGPTIMKGYFCNAEATATTLDSEGWLKTGDLCYFDEEGFLFVVDRIKELIKYKGYQVAPAELEELLLSNPEIADAAVIPYPDKEAGQVPMAFIVRKADSKLKEEDVKSFVSKQVAPYKKIRRVAFVTSIPKSASGKILRKDLIQ

SEQ ID NO: 85 - DNA, 1611 bp, Nageia nagi
atggacccaatgagtggtttctgtacctctaatggtgtctattactctaagagggagccatctgctcttccttctccacaccagaacatggacatcaccacttacgccttcagtcaccaccacaccactaacatcgcctttgtcgatgccccaaccggtaggagtttgtcttactctacccttaggaggaacgttaaagcccttgccgccggtttgcataggttgggagtccagaaggacgacgtcgtcttggttttgtctcctaacagtattgatattccttgtatttacatgggaatccttagtttgggagccatcttgaccaccgccaatcctttgaacaccgaggccgagatccaaaagcaagttgccgacagtaacccagccatcgtctttgccgccccagaattgcttgacaaagccagagccacccagaggccagtcgtcatcattggtgacgagaaccagactttgcctcacggttgtgtcgcctctcttcaagaattgttgcagagtccaatcgatggtgccccaccagttgaaatcaagcaagaggacaccgccacccttctttactcttctggtactaccggaaaagccaagggtgtcgttgccacccatagaaactacatcgccatgatggctggtcttgttaatacccccggtgacgacgtcgagaaggacgtctaccttttgatcatgcctcttttccatgtttatggtttctttagaatgatctggagtgttgccatgggtaacaccgtcgtcgtcatgccaaagtttgatttggcccagatgctttggaacatcgagaggtatagagtcacttgtatcccagctgctccacctattttcgtcgcccttgccaagtctcctatcgtcgaaaagtacgacttgagtagtttgcaaaggatcggttctggtggtgctgccttgggtaaagaggtcatcgaggagttcatgggtaggtttccaagggttgaagtcggtcaaggttacggtttgaccgaaagttgcggtgctgctaccttgaccgtttctgctgaagagaaaaagaagtacggaaccgccggtcttttgttggccaacatggaggccaagatcgtcaacaccgtcaccggaaagcctttgccaccaaataggaggggtgagttgtggcttaggggtccatgcatcatgaagggatattataacaataaggaagccaccactgctacccttgacagtgagggttggcttaagactggagacttgtgctatatcgacgaggagggattcctttttgttgtcgataggattaaagaattgattaagtacaaggccttccaagttgccccagctgagttggaggagcttcttttgagtcacccagagatcgccgactgcgccgttattccttatccagatgacgaggccggacagatcccaatggccttcatcgttagaaagagtggaagtaaattgaaacaagaggatgttcttagttttgtcagtaaacaagttgctccatataagaaaattagaagggttgcttttgtcaactctattccaaaaagtcaatctggtaaaattttgagaaaggagttcatccaacaaaccctttctacttaa

SEQ ID NO: 86 - protein, 536 aa, Nageia nagi
MDPMSGFCTSNGVYYSKREPSALPSPHQNMDITTYAFSHHHTTNIAFVDAPTGRSLSYSTLRRNVKALAAGLHRLGVQKDDVVLVLSPNSIDIPCIYMGILSLGAILTTANPLNTEAEIQKQVADSNPAIVFAAPELLDKARATQRPVVIIGDENQTLPHGCVASLQELLQSPIDGAPPVEIKQEDTATLLYSSGTTGKAKGVVATHRNYIAMMAGLVNTPGDDVEKDVYLLIMPLFHVYGFFRMIWSVAMGNTVVVMPKFDLAQMLWNIERYRVTCIPAAPPIFVALAKSPIVEKYDLSSLQRIGSGGAALGKEVIEEFMGRFPRVEVGQGYGLTESCGAATLTVSAEEKKKYGTAGLLLANMEAKIVNTVTGKPLPPNRRGELWLRGPCIMKGYYNNKEATTATLDSEGWLKTGDLCYIDEEGFLFVVDRIKELIKYKAFQVAPAELEELLLSHPEIADCAVIPYPDDEAGQIPMAFIVRKSGSKLKQEDVLSFVSKQVAPYKKIRRVAFVNSIPKSQSGKILRKEFIQQTLST

SEQ ID NO: 87 - DNA, 1608 bp, Dioon edule
atgtctggtatcgatccagccaacggttactgtgagtgcaacggtatttactattctaaaagggacccagttgctttgccaccagagcacgagaacatgggtgtcgtcacttttgttttctctgagaaccatgacggaaccgcccttattgacgctcctaccggtcataccttgagtcaccaccagcttaggaggaacgttatggctttgggtgctggattgcatgcccttggtattaggcaaggtgacgtcgtcttggtcttgtctccaaactctatcatgttgccttgcatccatttggccatcctttctgtcggagccattcttaccaccgccaatccacttagtacccccggtgaaatcgagaggcagatccaagattctaaggccttgatggtctttaccttgccacatcttatccctaaggtcagtcagttgccagccgtcttgatccagagtgacggtcacaagcacagtagttgcgtttctaccgtcgagcacttgttggatagatacagtgacgccagtgctcttccaagtgtcaacattagacagcatgacaccgccaccttgttgtacagtagtggtaccaccggtaagtctaagggtgttgtctcttctcatagaaattacatcgctattatcgccgaaagtagaatgaatgccaaaagaaaaggtgttagttctaatgtcagtttttgtactatgccaatgtttcacgttttcggttttttcttgaccaattctttgatcgccgtcggagacactatcgttgtcatgccaaagttcgacttggagaagatgttgtgggccgtcgaaaggtacagagttacctctttgccagctgccccacctatcttggtcgctcttgccaagagtagaatcgccgacaaatacgatttgtctagtttgcagttgatcggatctggaggagctcctttgggtaaggagttgatcgaggagttcgccgctagattcccaaccatcgaggtcactcaaggttacggacttaccgagagttctggtggtgtctcttacaccgaaggtaatgaagaaatcaagcactacggtaccgccggattgcttaacgccaatgtcgaggccaaggtcgttgatccagtctctggtaagcctttgccaccaaaccacagaggtgagctttggttgaggggaccaaccatcatgaagggttacttgggtaacgaagaggctactgcctctacccttaacagtgacggttggcttaagaccggagatttgtgttacatcgacgagaagggtttccttttcgtcgtcgatagacttaaagaattgatcaagtacaagtcttaccaagttgccccagctgagttggaggaattgcttttgtctcactctgagatctctgacgccgctgttatcccttacccagataacgaggccggtcagattccaatggcctacgtcgttagaaaaccatattctaaactttctgaagaggacgttatcaacttcgtcgccaaccaagttagtccatacaagaaggttagacaagttgctttcgtcagttctattcctaagagtccatctggaaaaatccttaggaaggatcttatccaacaagcttaa

SEQ ID NO: 88 - protein, 535 aa, Dioon edule
MSGIDPANGYCECNGIYYSKRDPVALPPEHENMGVVTFVFSENHDGTALIDAPTGHTLSHHQLRRNVMALGAGLHALGIRQGDVVLVLSPNSIMLPCIHLAILSVGAILTTANPLSTPGEIERQIQDSKALMVFTLPHLIPKVSQLPAVLIQSDGHKHSSCVSTVEHLLDRYSDASALPSVNIRQHDTATLLYSSGTTGKSKGVVSSHRNYIAIIAESRMNAKRKGVSSNVSFCTMPMFHVFGFFLTNSLIAVGDTIVVMPKFDLEKMLWAVERYRVTSLPAAPPILVALAKSRIADKYDLSSLQLIGSGGAPLGKELIEEFAARFPTIEVTQGYGLTESSGGVSYTEGNEEIKHYGTAGLLNANVEAKVVDPVSGKPLPPNHRGELWLRGPTIMKGYLGNEEATASTLNSDGWLKTGDLCYIDEKGFLFVVDRLKELIKYKSYQVAPAELEELLLSHSEISDAAVIPYPDNEAGQIPMAYVVRKPYSKLSEEDVINFVANQVSPYKKVRQVAFVSSIPKSPSGKILRKDLIQQA

SEQ ID NO: 89 - DNA, 1614 bp, Fokienia hodginsii
atggagcagatggaggaattgaagaggtgcccagctaactaccctcctttcaccccagtcggttttgtcgagcttgccgctaccgtctactctgactgcaccagtatcgtctacaataccactagattcacttgtagacagttgagtagtgccttggctcttaggaacatctgcagaggtgacgtcgtcagtgttgttgcccctaacatcccagctatgtacgagatgcacttcgctatccctatgggtggagctgtccttaataatttgaacactaggttggatgctagaaccatggctgcccaaatctctcactgtgagccaaagatcatttttgttgattatcaatttttgtctgtcgttaatgaaaccttgtctttgcttaagcataagccattgttggtcgtcatcgaggagatggagaacggaaaggagatcggtagtggtgccgccttgacctacgagggtttgcttagggagggagacccagagttcgaaattagatggccagaagacgaatggcaagctgccgtccttaactacacttctggaaccacctctgccccaaaaggtgttgtccaatctcacagaggaatctacgccatggcccttgacaaccttaccatgtggcagatgggaaggagaccagtctacctttggaccttggccatgttccatgctaacggttggtgcttgccttggactttggctgctgtcggaggtaccaacatctgcttgaggaagttcgacagtaaaattattttcgattctatcgccgagcataaggtcacccacatgtgtggagctccagtcgtcttgtctatgatggccaatgccgacccagctgacagaaagcacttgagttaccaagttgagattttgactgccggagccccacctccagctgccatcttgtggaagattgaggagttgggtttctctatcacccacggttatggtttgaccgagaccgccggtttggtcgtttcttgcgcttggaaaaccgaatgggatggattgcccggtaaggagaaggctagattgaagagtaggcaaggtgttagaaacttgagtcttgccgaggtcgacgttaagaacccagttaccatggctagtgtcgctagagacggtgtccaaatgggtgaggtcatgatgaggggtgcctctgttatgaagggatacttgaagaatgaggagatgaccgcttctgccatggagggtggttggtttaggaccggagatgttgctgtcgtccacccagatggatacatcgagatcaaggatagaagtaaggacgtcatcatctctggtggtgaaaacatctctagtgtcgaggtcgagtctgtcctttacagtcacccatctgtcgtcgaagctgctgtcgttgccagaccagatccattctggggtgagactccatgcgccttcgtcagtgtcaagaagcacggtggtaagggaaatagagaagtcttgtctgaggccgagatcatcgagttctgtagaaagcacttggcccactttatggctccaaagagtgtcatcttcatgcaagaattgcctaagactagtaccggtaagatccaaaaatttgtccttagggaaatggctaaataa

SEQ ID NO: 90 - protein, 537 aa, Fokienia hodginsii
MEQMEELKRCPANYPPFTPVGFVELAATVYSDCTSIVYNTTRFTCRQLSSALALRNICRGDVVSVVAPNIPAMYEMHFAIPMGGAVLNNLNTRLDARTMAAQISHCEPKIIFVDYQFLSVVNETLSLLKHKPLLVVIEEMENGKEIGSGAALTYEGLLREGDPEFEIRWPEDEWQAAVLNYTSGTTSAPKGVVQSHRGIYAMALDNLTMWQMGRRPVYLWTLAMFHANGWCLPWTLAAVGGTNICLRKFDSKIIFDSIAEHKVTHMCGAPVVLSMMANADPADRKHLSYQVEILTAGAPPPAAILWKIEELGFSITHGYGLTETAGLVVSCAWKTEWDGLPGKEKARLKSRQGVRNLSLAEVDVKNPVTMASVARDGVQMGEVMMRGASVMKGYLKNEEMTASAMEGGWFRTGDVAVVHPDGYIEIKDRSKDVIISGGENISSVEVESVLYSHPSVVEAAVVARPDPFWGETPCAFVSVKKHGGKGNREVLSEAEIIEFCRKHLAHFMAPKSVIFMQELPKTSTGKIQKFVLREMAK

SEQ ID NO: 91 - DNA, 1605 bp, Cryptomeria japonica
atggctagtgatttgaatccttctagtggattcagtatggctactggtatttattattctaagagagatccaatccctatcccaccaccacaccagcatcttgatttgaccacctacgttttcagtcacccacacaacaccgagatcgccatcatcgatgcccaaagtaacgcccagttgtcttatagagccttgaggcacaatgtcagagctttggccaccggtttgcagaggttgggtattagaaaaagggatgtcgtcttggttatcttgccaaatatcattcatgttccatctatctatcttgccatcgtctctatcggtgccattttgaccaccgccaacccattgaacaccgagaccgagattagaaaacaagttgccgactctaatccagttttggctttcgctgccccagaattcgctcataaggctagagccgccaagttgccagtcgtccttacccagtctaccaaccttaccgctaacgatagtgagtacgtcgccacccttcacgagttgttccagtctgacgtcaacgatttccagccagttgacatccagcaagaggacaccgccaccttgttgtacagtagtggtaccaccggaaagtctaagggtgtcgtcgctacccatagaaaccatatcgccatggtcgccggatttgtcaatagaattgacactcaaaatatcatcaccttgtgcaccatgcctttgttccacgtctacggattcttctatagtgtttcttctgtcgccaccggaaccaagcttgtcttgatggccaagttcgactttgcccaaatgcttgccaacgtcgagaggtacaaggtcactagtttgccagttgccccaccaatcttcgttgccttgaccaagagtcctatcgtcaccaaatacgatttgtcttctttgaagagaatcggttctggtggtgctccacttggtaaggagaccatcgacgagttcatggctcttttccctaacattgaggtctctcaaggttatggattgaccgagtctagtggtgccgttaccttcacctctaccggagaggagaagaagaagtacggtaccgccggtcttttggccgctaacgttgaggctaagatcgtcgacgttgtcagtaaaaaggccttgccaccaaaccagaggggtgagctttggttgaggggacctaccatcatgaagggttacttttctaacgacgaggccaccgctaccaccttggactctgagggttggttgaagaccggtgacctttgctacttcgacgaggaaggttttctttttgttgttgatagaattaaggaacttatcaagtacaaaggttatcaagttgccccagccgagcttgaggagcttcttttgagtaacccagagattagtgacgccgccgttatcccttacccagataaggaggccggtcagatccctatgagtttcatcgttagaaaggccggatctaaacttaacgaagaggacgtcaaaagttttgttagtaaacaagttgccccatataagaaaattaggagggttgccttcgtcaccagtatccctaagtctgcttctggtaagattttgagaaaagatcttatccaataa

SEQ ID NO: 92 - protein, 534 aa, Cryptomeria japonica
MASDLNPSSGFSMATGIYYSKRDPIPIPPPHQHLDLTTYVFSHPHNTEIAIIDAQSNAQLSYRALRHNVRALATGLQRLGIRKRDVVLVILPNIIHVPSIYLAIVSIGAILTTANPLNTETEIRKQVADSNPVLAFAAPEFAHKARAAKLPVVLTQSTNLTANDSEYVATLHELFQSDVNDFQPVDIQQEDTATLLYSSGTTGKSKGVVATHRNHIAMVAGFVNRIDTQNIITLCTMPLFHVYGFFYSVSSVATGTKLVLMAKFDFAQMLANVERYKVTSLPVAPPIFVALTKSPIVTKYDLSSLKRIGSGGAPLGKETIDEFMALFPNIEVSQGYGLTESSGAVTFTSTGEEKKKYGTAGLLAANVEAKIVDVVSKKALPPNQRGELWLRGPTIMKGYFSNDEATATTLDSEGWLKTGDLCYFDEEGFLFVVDRIKELIKYKGYQVAPAELEELLLSNPEISDAAVIPYPDKEAGQIPMSFIVRKAGSKLNEEDVKSFVSKQVAPYKKIRRVAFVTSIPKSASGKILRKDLIQ

SEQ ID NO: 93 - DNA, 1590 bp, Dacrydium balansae
atgggttattgtgccgccaccggtattttccatacccttagagacccacttcctcttcctccaccacaccagaatttggacatcaccaccttcgttttctctcaccaccacaccaccgacatcgcccttatcgatgctcctaccggatgcagtcttagttacgctgccttgaggaggaacgtcaagtctttgtctgccgccttgcacagtcttggtattagaaagggtgacgttgtccttgtcctttctcctaattacatccatattccatgcatctacatggccatcttcagtgtcggagccattcttaccaccgccaaccctcttaacaccgagtctgagatcttgaaccaagttaccgacagtaacccagctatcgtttttgccgctccagatcttgtcccaaaggtcagagctaccagaaggccaatcgttgtcatccacaccgagaagggagaacaacacggttgcgttgccaccttgtgggaattgcttcagtctagttttaaggaggcccctaaggtcgacattcagcaagatgataccgccaccttgctttacagttctggtaccaccggtaagagtaagggtgtcatcggttctcataggaactttatctctatggtcgccgcccttggttctggtccaaacgaatggaacatcgtcgtcatgatgactatgccaatgtttcatgtttatggttttttgttcgctatgtctttcttggccagtggttctaccattgttgttatgcctaaattcgacttcgctcttatgttgtggtctgtccagaggtatagagttaggtacttgcctaccagtcctccattgttcgtcgccttgaccaagtctccaatggttgagaaatatgaccttaccagtttggagagggtcgcttctggaggtgcccctcttggtaaagaagtcatcgacgagttcgctagaagattccctaacgtcgaggtcgctcaaggttacggtttgaccgagtcttgcggagccgtcactttcacttcttctgccgaggagaaaaagaaatatggatctgctggtttgcttgccgccaatatggaggctaagatcgtcgacaccgtcactggtaaagccttgccaccaaaccagaggggtgagttgtggttgaggggtccatctatcatgaagggttactataatgacgaggaggccacccttgctaccttggacagtgagggttggcttaagaccggagacttgtgctacatcgacgacgagggtttcttgttcgttgtcgatagaatcaaagagcttattaaatataaggctttccaagttgctccagccgagcttgaggagttgttgttgagtcacaccgagatcgccgactgcgccgttatcccatatccagacgacaagaccggtcagatccctatggccttcatcgtcagaaagtctggaagtaaagtcaaggaggaggacgttatgagtttcgtcgccaagcaagttgccccatacaagaagatcagaagggtcgccttcgtcaactctatccctaaatctcaaagtggaaaaattcttaggaaagacttgatccaacagaccttgtctgtttaa

SEQ ID NO: 94 - protein, 529 aa, Dacrydium balansae
MGYCAATGIFHTLRDPLPLPPPHQNLDITTFVFSHHHTTDIALIDAPTGCSLSYAALRRNVKSLSAALHSLGIRKGDVVLVLSPNYIHIPCIYMAIFSVGAILTTANPLNTESEILNQVTDSNPAIVFAAPDLVPKVRATRRPIVVIHTEKGEQHGCVATLWELLQSSFKEAPKVDIQQDDTATLLYSSGTTGKSKGVIGSHRNFISMVAALGSGPNEWNIVVMMTMPMFHVYGFLFAMSFLASGSTIVVMPKFDFALMLWSVQRYRVRYLPTSPPLFVALTKSPMVEKYDLTSLERVASGGAPLGKEVIDEFARRFPNVEVAQGYGLTESCGAVTFTSSAEEKKKYGSAGLLAANMEAKIVDTVTGKALPPNQRGELWLRGPSIMKGYYNDEEATLATLDSEGWLKTGDLCYIDDEGFLFVVDRIKELIKYKAFQVAPAELEELLLSHTEIADCAVIPYPDDKTGQIPMAFIVRKSGSKVKEEDVMSFVAKQVAPYKKIRRVAFVNSIPKSQSGKILRKDLIQQTLSV

SEQ ID NO: 95 - DNA, 1614 bp, Oncotheca balansae
atgccagccaactaccctcctttgacccctatcggtttcatccagagagctgccaccgtttacggtgactgtagttctatcgtctacgataccaccagattcacttggttccagacctttcagaggtgtaggaagttggccagtgccttgagtagtaggaaccttagtagaggtgacgtcgtctctgtcgttgctccaaacgtcccagccatgtacgagatgcatttcgccgttccaatggccggtgccgttttgaacaacgtcaacatcagacttgatgccggaaccatggctgcccaattctctcactgcgaacctaagttcgtcttcgtcgactaccagtttttgccaatcattagagacgcccttagtaggatcgagagaaacccatgcgtcgtcgttatcgaggagcttcacaacggtagggagatccctacttctggtggaatcacctacgagggattgatcggtgagggtgacgccgactttgaaattagatggccagaagacgagtggcaagctgctgttttgaattacacctctggtaccaccagtgctccaaaaggtgtcgtccattgccataggggtatctacgctatggccatggacaacgtccttatgtggggattgaagatgcagccagttttcttgtggactttggccatgttccacgccaatggttggtgtttcccatgggctttggccgctgtcggaggtaccagtatctgccttaggaagtttgacgctaagattgtcttcgagagtatggccgaacacggtgttacccatatgtgcggtgctccagtcgtccttagtatgatggccaatgcccagcctagtgagagaaagccacttgccggtagggtcgaaatcttgactgctggatctccaccaccagctgccattttgtggaaggtcgaggacatgggtttcagtgtcacccacggttacggtttgactgagaccgccggtttggttgtctcttgcgcttggaaaggtgaatgggacaagcttccagccgaagagagagctagattgaaggctagacaaggtgttagaaacgtttctaccgctcaagttgacgtcaaagagccaagtagtatggcctctgtcgctagggacggtgtccaaatgggtgaggtcatgattagaggtggtagtgtcatgaagggataccttaaaaatcagcaagctaccgctagagctatggacggtggttggttcagaaccggtgatgttgccgtcgtccacccagacggttatttggagatcaaggatagaagtaaagatattattatctctggtggtgagaacatctcttctgtcgaagttgagtctgttttgtacagtcaccctttggtcgttgaagctgctgtcgttgctaggccagatccattctggggtgaaaccccatgcgctttcgtctctattaataacaattctaaagaggtcttgtctgaggcccaagttatcagtttctgtagacagagaatcgcccacttcatggccccaaagagtgtcatcttcatggaggagcttccaaagacctctactggtaagattaaaaaatacttgttgagggagatggccaagtcttttgcccaacctagataa

SEQ ID NO: 96 - protein, 537 aa, Oncotheca balansae
MPANYPPLTPIGFIQRAATVYGDCSSIVYDTTRFTWFQTFQRCRKLASALSSRNLSRGDVVSVVAPNVPAMYEMHFAVPMAGAVLNNVNIRLDAGTMAAQFSHCEPKFVFVDYQFLPIIRDALSRIERNPCVVVIEELHNGREIPTSGGITYEGLIGEGDADFEIRWPEDEWQAAVLNYTSGTTSAPKGVVHCHRGIYAMAMDNVLMWGLKMQPVFLWTLAMFHANGWCFPWALAAVGGTSICLRKFDAKIVFESMAEHGVTHMCGAPVVLSMMANAQPSERKPLAGRVEILTAGSPPPAAILWKVEDMGFSVTHGYGLTETAGLVVSCAWKGEWDKLPAEERARLKARQGVRNVSTAQVDVKEPSSMASVARDGVQMGEVMIRGGSVMKGYLKNQQATARAMDGGWFRTGDVAVVHPDGYLEIKDRSKDIIISGGENISSVEVESVLYSHPLVVEAAVVARPDPFWGETPCAFVSINNNSKEVLSEAQVISFCRQRIAHFMAPKSVIFMEELPKTSTGKIKKYLLREMAKSFAQPR

SEQ ID NO: 97 - DNA, 1644 bp, Taxus x media
atggaggaattgaagaggtgtccagctaacgacccaccacttaccccagttggtttcattgaaagggccgctaccgtctatggtgactgcatcagtgtcgtctatagatctacctctttcacttggagtcagaccttcaacagatgtagaaagttggctagtgctctttcttctaggaacattctttctggtgacgtcgtttctgtcgttgctccaaacgtcccagccttgtacgagatgcacttcggtgtcccaatggccggtgccgttttgaacaccgtcaacattagattggatgccagaaccatggccgcccaattcactcactgcgagcctaagcttgtcttcgccgattatcaattcatcccattggtcatggaggcccttagtggtcttgagcacaaaccttgcgtcgtcttgatcgaggaaaccgacaatgccagagagactgctacctctggtgccttgacctatgagggtttgatcggtgagggtgacccagagttcgaaatcagatggccagaggatgagtggcagagtgccgtccttaactacacctctggtactacctctgcccctaagggtgtcgtccactgccatagaggattgtacgtcatggccatggacatccttgtcatgtctggtatgagaagtcaatctgtcttcttgtggaccttgccaatgttccacgccaacggatggtgtttcacttgggctattgctgccgtcggtggtaccaacatctgtcttagaaaattcgacgctaaaatcattttcgacgccatcaccgatcatagagtcacccatttgtgcgctgccccagtcgtcttgtctatgatggctaacgcccacccttctgagaggaagcctttgcaaggtaaggtcgagatcttcaccggaggaagtccacctccagccgctatcttgttgaagaccgagcagttgggtttctctgtcacccatgcttacggtcttaccgagactgctggtgttgtcgttgcttgtgcttggaaaagggagtgggataagttgcccggtcaagaaagggctaggatgaaggctagacaaggtgttagaagtccatctaccgcccaagttgacgttaaagacccagcttctatggccagtgtcgctagagacggagttcagatgggagagttgatgattagaggagcctctgtcatgaagggttacttgaagaacgagcagatgaccgccagagctatggatggtggttggttcagaaccggtgatgtcgccgtcgttcacccagatggttacttggaaatcaaggataggtctaaagatgttattattagtggtggtgaaaacatctctagtgttgaggttgaatctgttttgtacagtcatccattgatcatggaggccgccgttgttatgagaccagacgctttctggggagagaccccttgcgcctttgtctctatcaataataacagtgatgcctctgaagccttgtctgaggcccaagttatctctttttgtagagagaggatggcccacttcatggctcctaagtctgtcatttttatgaagaaacttccaaaaaccagtaccggtaaaatccaaaaattcgtccttagggagatggctaagacccttagtaggccttctgccatgtaa

SEQ ID NO: 98 - protein, 547 aa, Taxus x media
MEELKRCPANDPPLTPVGFIERAATVYGDCISVVYRSTSFTWSQTFNRCRKLASALSSRNILSGDVVSVVAPNVPALYEMHFGVPMAGAVLNTVNIRLDARTMAAQFTHCEPKLVFADYQFIPLVMEALSGLEHKPCVVLIEETDNARETATSGALTYEGLIGEGDPEFEIRWPEDEWQSAVLNYTSGTTSAPKGVVHCHRGLYVMAMDILVMSGMRSQSVFLWTLPMFHANGWCFTWAIAAVGGTNICLRKFDAKIIFDAITDHRVTHLCAAPVVLSMMANAHPSERKPLQGKVEIFTGGSPPPAAILLKTEQLGFSVTHAYGLTETAGVVVACAWKREWDKLPGQERARMKARQGVRSPSTAQVDVKDPASMASVARDGVQMGELMIRGASVMKGYLKNEQMTARAMDGGWFRTGDVAVVHPDGYLEIKDRSKDVIISGGENISSVEVESVLYSHPLIMEAAVVMRPDAFWGETPCAFVSINNNSDASEALSEAQVISFCRERMAHFMAPKSVIFMKKLPKTSTGKIQKFVLREMAKTLSRPSAM

SEQ ID NO: 99 - DNA, 60 bp, Artificial
  Synthetic polynucleotide
aacatctttaacatacacaaacacatactatcagaatacaatgggaaaaaattataagtc

SEQ ID NO: 100 - DNA, 60 bp, Artificial
  Synthetic polynucleotide
aaaaacgtgttttttggactagaaggcttaatcaaaagctttactcaaaatgactaaact

SEQ ID NO: 101 - DNA, 1638 bp, Cannabis sativa
atgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 102 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 103 - DNA, 1554 bp, Artificial
  Synthetic polynucleotide
aatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaa

SEQ ID NO: 104 - protein, 517 aa, Artificial
  Synthetic polypeptide
NPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

(19) United States
(12) Patent Application Publication
(10) Pub. No.: US 2022/0186231 A1
(43) Pub. Date: Jun. 16, 2022

(54) RECOMBINANT ACYL ACTIVATING ENZYME (AAE) GENES FOR ENHANCED BIOSYNTHESIS OF CANNABINOIDS AND CANNABINOID PRECURSORS

(71) Applicants: Willow Biosciences, Inc., Calgary (CA); Epimeron USA, Inc., Mountain View, CA (US)

(72) Inventors: Mathias SCHUETZ, Langley (CA); Michal PYC, Langley (CA)

(73) Assignees: Willow Biosciences, Inc., Calgary (CA); Epimeron USA, Inc., Mountain View, CA (US)

(21) Appl. No.: 17/548,208

(22) Filed: Dec. 10, 2021

Related U.S. Application Data
(60) Provisional application No. 63/124,526, filed on Dec. 11, 2020.

(51) Int. Cl. C12N 15/52; C12N 1/18; C12P 7/42; C12N 9/88; C12N 9/10; C12N 9/00; C12N 9/02

(52) U.S. Cl. CPC C12N 15/52; C12N 1/18; C12P 7/42; C12N 9/88; C12Y 404/01026; C12N 9/1029; C12Y 121/03008; C12N 9/93; C12Y 602/01002; C12N 9/1085; C12N 9/0004; C12Y 121/03007; C12Y 203/01206

(57) ABSTRACT

The present disclosure provides recombinant host cells comprising a pathway capable of producing a cannabinoid and/or cannabinoid precursor, wherein the pathway comprises an enzyme AAE from a source organism other than Cannabis sativa, such as Humulus lupulus. The disclosure also provides methods of using the host cells to produce rare cannabinoids and/or rare cannabinoid precursors.

### FIELD

The present disclosure relates generally to recombinant host cells with a cannabinoid biosynthesis pathway comprising gene encoding an AAE enzyme from a source organism other than Cannabis sativa, such as Humulus lupulus, to enhance the ability of the host cell to produce cannabinoids, such as CBGA and CBGVA, and methods for using the recombinant host cells and genes for cannabinoid production.

### REFERENCE TO SEQUENCE LISTING

The official copy of the Sequence Listing is submitted concurrently with the specification as an ASCII formatted text file via EFS-Web, with a file name of “13421-008WO1_SeqList_ST25.txt”, a creation date of Dec. 10, 2021, and a size of 339,501 bytes. The Sequence Listing filed via EFS-Web is part of the specification and is incorporated in its entirety by reference herein.

### BACKGROUND

Cannabinoids are a class of compounds that act on endocannabinoid receptors and include the phytocannabinoids naturally produced by Cannabis sativa. Cannabinoids include the more prevalent and well-known compounds, Δ⁹-tetrahydrocannabinol (THC), cannabidiol (CBD), as well as 80 or more less prevalent cannabinoids, cannabinoid precursors, related metabolites, and synthetically produced derivative compounds. Cannabinoids are increasingly used to treat a range of diseases and conditions such as multiple sclerosis and chronic pain. Current large-scale production of cannabinoids for pharmaceutical or other use is through extraction from plants. These plant-based production processes, however, have several challenges including susceptibility of the plants to inconsistent production caused by variance in biotic and abiotic factors, difficulty reproducing identical cannabinoid accumulation profiles, and difficulty in producing a single cannabinoid compound with purity high enough for pharmaceutical applications. While some cannabinoids can be produced as a single pure product via chemical synthesis, these processes have proven very costly and too costly for large-scale production.

There are numerous rare cannabinoids produced by C. sativa in low abundance, such as the varin cannabinoids, cannabigerovarin (CBGV), tetrahydrocannabivarin (THCV), cannabidivarin (CBDV), and cannabichromevarin (CBCV). The rare cannabinoids produced by C. sativa are believed also to have medical uses but have not been as thoroughly investigated due to the difficulty of obtaining them in amounts sufficient and cost-effective for carrying out clinical trials.

More economical biosynthetic approaches to cannabinoid production are being developed using microbial hosts. These processes have the potential to be robust, scalable, and capable of producing single cannabinoid compound with higher purity compared to other current processes. Several biosynthetic systems for cannabinoid compound have been reported (see e.g., WO2019071000, WO2018200888, WO2018148849, WO2019014490, US20180073043, US20180334692, and WO2019046941). However, these biosynthetic systems are not efficient in the biosynthesis of rare cannabinoid compounds, such as the varin cannabinoids.

There exists a need for improved biosynthetic systems and methods for the production of rare cannabinoid compounds. In particular, there is a need to improve the performance of recombinant microbial hosts for the biosynthesis of rare cannabinoid compounds such as the varin series of cannabinoids.

### SUMMARY

The present disclosure relates to recombinant host cells comprising a pathway capable of producing rare cannabinoids, such as the varin cannabinoid, cannabigerovarinic acid (CBGVA), and/or producing rare cannabinoid precursor compounds, such as divarinic acid (DA). The present disclosure also relates to the specific enzymes in the pathway (and the recombinant nucleic acids encoding them) that facilitate enhanced production of rare cannabinoids and/or their rare cannabinoid precursor compounds. The present disclosure also relates to methods using the recombinant host cells, pathways, enzymes, and nucleic acids, for the production of rare cannabinoids, such as varin cannabinoids, starting from either divarinic acid (“DA”), and/or the butyric acid (“BA”) as precursor feedstock. The disclosure also relates to compositions comprising nucleic acids encoding the heterologous genes that provide enhanced production of rare cannabinoids. This summary is intended to introduce the subject matter of the present disclosure, but does not cover each and every embodiment, combination, or variation that is contemplated and described within the present disclosure. Further embodiments are contemplated and described by the disclosure of the detailed description, drawings, and claims.

In at least one embodiment, the present disclosure provides a recombinant host cell which produces a cannabinoid precursor and/or a cannabinoid, wherein the cell comprises a pathway of enzymes AAE, OLS, OAC, and optionally, PT4, wherein the AAE has an amino acid sequence of at least 70% identity to a sequence selected from: CCL3 (SEQ ID NO: 24), CH3 (SEQ ID NO: 30), TM4 (SEQ ID NO: 16), CCL2 (SEQ ID NO: 18), CM1 (SEQ ID NO: 20), DA1 (SEQ ID NO: 22), AA1 (SEQ ID NO: 26), WC1 (SEQ ID NO: 28), CH2 (SEQ ID NO: 32), PA1 (SEQ ID NO: 34), and TM5 (SEQ ID NO: 36). In at least one embodiment, the AAE has an amino acid sequence of at least 70%, at least 75%, at least 80%, at least 85%, at least 90%, at least 95%, at least 98%, or greater identity to a sequence selected from SEQ ID NO: 16, 18, 20, 22, 24, 26, 28, 30, 32, 34, and 36.

In at least one embodiment, the present disclosure provides a recombinant host cell which produces olivetolic acid (OA) and/or divarinic acid (DA) when cultured in the presence of hexanoic acid (HA) and/or butyric acid (BA), wherein the cell comprises a pathway of enzymes AAE, OLS, and OAC, wherein AAE is not from C. sativa; optionally, wherein the recombinant host cell AAE has an amino acid sequence of less than 60% identity to SEQ ID NO: 2. In at least one embodiment, the AAE is from a plant source selected from Amentotaxus argotaenia; Callitris macleayana; Cephalotaxus harringtonia; Diselma archeri; Humulus lupulus; Prumnopitys andina; Taxus x media; and Widdringtonia cedarbergensis. In at least one embodiment, the AAE has an amino acid sequence of at least 70%, at least 75%, at least 80%, at least 85%, at least 90%, at least 95%, at least 98%, or greater identity to a sequence selected from SEQ ID NO: 16, 18, 20, 22, 24, 26, 28, 30, 32, 34, and 36.

In at least one embodiment of the recombinant host cell, the pathway catalyzes the reactions (i)(a)-(iii)(a) and/or (i)(b)-(iii)(b):

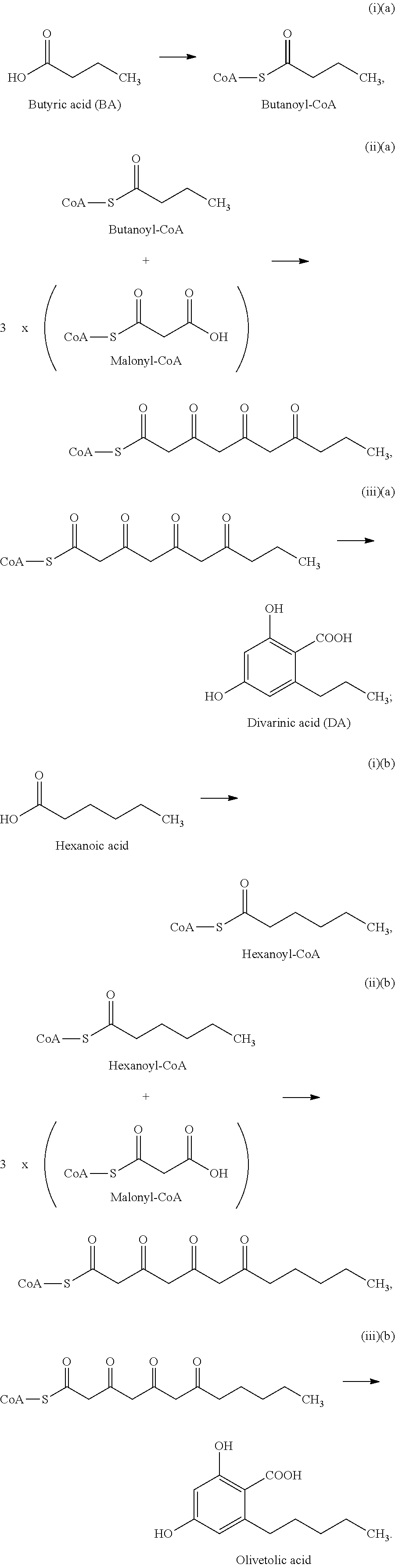

In at least one embodiment of the recombinant host cell, the pathway enzymes OLS, and OAC have an amino acid sequence of at least 90% identity to SEQ ID NO: 4 (OLS), and SEQ ID NO: 6 (OAC), respectively.

In at least one embodiment of the recombinant host cell, the pathway catalyzes reaction (iv)(a) and/or (iv)(b):

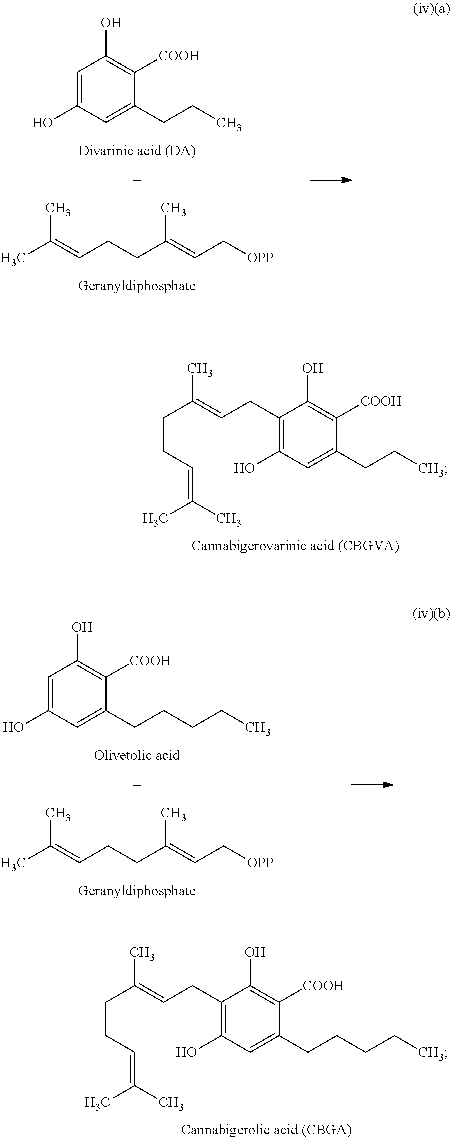

In at least one embodiment of the recombinant host cell, the pathway comprises the enzyme PT4; optionally, wherein the PT4 has an amino acid sequence of at least 90% identity to SEQ ID NO: 8 or 10 (PT4) respectively.

In at least one embodiment of the recombinant host cell, the recombinant host cell pathway further comprises an enzyme capable of catalyzing a reaction (v)(a), (vi)(a), (vii)(a), (v)(b), (vi)(b), and/or (vii)(b):

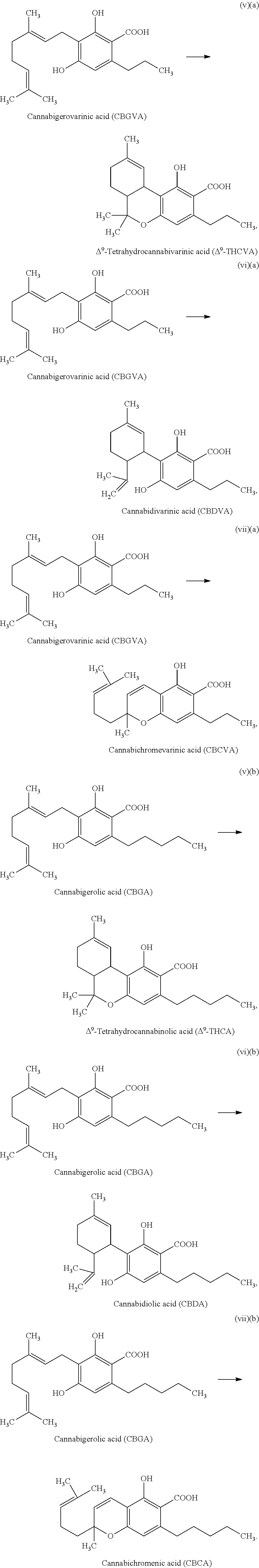

In at least one embodiment of the recombinant host cell, the pathway comprises an enzyme THCA synthase, CBDA synthase, and/or CBCA synthase; optionally, the enzyme CBDA synthase having an amino acid sequence of at least 90% identity to SEQ ID NO: 12 or 14, and/or the enzyme THCA synthase having at least 90% identity to SEQ ID NO: 102 or 104.

In at least one embodiment of the recombinant host cell: (a) the cell produces divarinic acid (DA) and/or cannabigerovarinic acid (CBGVA) when cultured in the presence of butyric acid (BA); (b) the cell produces olivetolic acid (OA) and/or cannabigerolic acid (CBGA) when cultured in the presence of hexanoic acid (HA); (c) the amount of DA and/or CBGVA the cell produces when cultured in the presence of BA is increased relative to the amount of DA and/or CBGVA produced by a control cell comprising a pathway of enzymes AAE, OLS, and OAC, wherein the control cell AAE is AAE1 from C. sativa comprising the amino acid sequence of SEQ ID NO: 2; (d) the amount of OA and/or CBGA the cell produces when cultured in the presence of HA is increased relative to the amount of OA and/or CBGA produced by a control cell comprising a pathway of enzymes AAE, OLS, and OAC, wherein the control cell AAE is AAE1 from C. sativa comprising the amino acid sequence of SEQ ID NO: 2; and/or (e) the amount of DA, CBGVA, OA, and/or CBGA produced by the cell is increased by at least 1.1-fold, at least 1.2-fold, at least 1.5-fold, at least 2-fold, at least 3-fold, at least 5-fold, at least 10-fold, or more relative to the control cell comprising a pathway of enzymes AAE, OLS, and OAC, wherein the control cell AAE is AAE1 from C. sativa comprising the amino acid sequence of SEQ ID NO: 2.

In at least one embodiment, the recombinant host cell of the present disclosure, the cell produces a cannabinoid selected from cannabigerolic acid (CBGA), cannabigerol (CBG), cannabidiolic acid (CBDA), cannabidiol (CBD), Δ9-tetrahydrocannabinolic acid (Δ9-THCA), Δ9-tetrahydrocannabinol (Δ9-THC), Δ8-tetrahydrocannabinolic acid (Δ8-THCA), Δ8-tetrahydrocannabinol (Δ8-THC), cannabichromenic acid (CBCA), cannabichromene (CBC), cannabinolic acid (CBNA), cannabinol (CBN), cannabidivarinic acid (CBDVA), cannabidivarin (CBDV), Δ9-tetrahydrocannabivarinic acid (Δ9-THCVA), Δ9-tetrahydrocannabivarin (Δ9-THCV), cannabidibutolic acid (CBDBA), cannabidibutol (CBDB), Δ9-tetrahydrocannabutolic acid (Δ9-THCBA), Δ9-tetrahydrocannabutol (Δ9-THCB), cannabidiphorolic acid (CBDPA), cannabidiphorol (CBDP), Δ9-tetrahydrocannabiphorolic acid (Δ9-THCPA), Δ9-tetrahydrocannabiphorol (Δ9-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

In at least one embodiment, the recombinant host cell of the present disclosure is capable of producing a varin cannabinoid selected from cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabichromevarinic acid (CBCVA), cannabichromevarin (CBCV), cannabigerovarinic acid (CBGVA), cannabigerovarin (CBGV), and any combination thereof.

In at least one embodiment of the present disclosure, the source organism of the recombinant host cell is selected from Saccharomyces cerevisiae, Yarrowia lipolytica, Pichia pastoris, and Escherichia coli.

In at least one embodiment, the recombinant host cell is Saccharomyces cerevisiae and the gene encoding the AAE enzyme is under the control of an ALD6 promoter. In at least one embodiment, the recombinant host cell is Saccharomyces cerevisiae and the cell comprises at least three copies of a gene encoding the AAE enzyme; optionally, wherein each copy is under the control of an ALD6 promoter.

In at least one embodiment, the present disclosure also provides a method for producing divarinic acid comprising: (a) culturing in a suitable medium comprising butyric acid (BA) a recombinant host cell of the present disclosure; and (b) recovering the produced divarinic acid (DA).

In at least one embodiment, the present disclosure also provides a method for producing a cannabinoid precursor and/or a cannabinoid comprising: (a) culturing a recombinant host cell of the present disclosure in a suitable medium comprising butyric acid (BA) and/or hexanoic acid (HA); and (b) recovering the produced divarinic acid (DA), cannabigerovarinic acid (CBGVA), olivetolic acid (OA), and/or cannabigerolic acid (CBGA). In at least one embodiment, the method can further comprise contacting a cell-free extract of a culture of a recombinant host cell of the present disclosure with a biocatalytic reagent or chemical reagent.

In at least one embodiment, the present disclosure also provides a method for producing a cannabinoid precursor and/or a cannabinoid comprising: (a) culturing in a suitable medium comprising butyric acid (BA) and/or hexanoic acid (HA), a recombinant host cell comprising a pathway of enzymes AAE, OLS, and OAC, wherein the AAE has an amino acid sequence of at least 70%, at least 75%, at least 80%, at least 85%, at least 90%, at least 95%, at least 98%, or greater identity to a sequence selected from SEQ ID NO: 16, 18, 20, 22, 24, 26, 28, 30, 32, 34, and 36; and (b) recovering the produced cannabinoid precursor and/or a cannabinoid. In at least one embodiment, the method can further comprise contacting a cell-free extract of a culture of a recombinant host cell of the present disclosure with a biocatalytic reagent or chemical reagent.

In at least one embodiment, the present disclosure also provides a method for making a recombinant host cell for producing a cannabinoid and/or a cannabinoid precursor, wherein the method comprises introducing into a host cell a set of nucleic acids that encode a pathway of enzymes AAE, OLS, and OAC, wherein the AAE is not AAE1 from C. sativa, and wherein the host cell produces divarinic acid (DA) when cultured in the presence of butyric acid (BA). In at least one embodiment, the AAE has an amino acid sequence of less than 90% identity, less than 80% identity, less than 70% identity, or less than 60% identity to AAE1 from C. sativa of SEQ ID NO: 2. In at least one embodiment, the AAE is from a plant source selected from Amentotaxus argotaenia; Callitris macleayana; Cephalotaxus harringtonia; Diselma archeri; Humulus lupulus; Prumnopitys andina; Taxus x media; and Widdringtonia cedarbergensis; optionally, wherein the AAE has an amino acid sequence of at least 70%, at least 75%, at least 80%, at least 85%, at least 90%, at least 95%, at least 98%, or greater identity to a sequence selected from SEQ ID NO: 16, 18, 20, 22, 24, 26, 28, 30, 32, 34, and 36.

### BRIEF DESCRIPTION OF THE DRAWINGS

A better understanding of the novel features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 depicts an exemplary four enzyme cannabinoid pathway capable of converting hexanoic acid (HA) to the cannabinoid precursor, olivetolic acid (OA), and then further converting OA to the cannabinoid, cannabigerolic acid (CBGA). The four enzymes catalyzing the steps in the pathway are AAE, OLS, OAC, and PT. The present disclosure provides AAE enzymes from source organisms other than C. sativa capable of acting in such a cannabinoid pathway in a recombinant host cell.

FIG. 2 depicts three exemplary two step pathways for converting the cannabinoid, CBGA, to one or more of the cannabinoids, Δ⁹-THCA, CBDA, and/or CBCA, and then, optionally, further converting them to the decarboxylated cannabinoids, Δ⁹-THC, CBD, and/or CBC. The first conversion from CBGA to Δ⁹-THCA, CBDA, and/or CBCA can be catalyzed by a cannabinoid synthase, CBDA synthase (CBDAS), THCA synthase (THCAS) and/or CBCA synthase (CBCAS), respectively. As described elsewhere herein, in some embodiments the single cannabinoid synthase (e.g., CBDAS) is capable of catalyzing not only the conversion of CBGA to its preferred product (e.g., CBDAS preferentially converts CBGA to CBDA), but also converts CBGA to one or both of the other cannabinoid acid products, typically in lesser amounts.

FIG. 3 depicts an exemplary four enzyme pathway capable of converting butyric acid (BA) to the rare cannabinoid precursor, divarinic acid (DA), and then further converting DA to the rare cannabinoid, cannabigerovarinic acid (CBGVA). The four enzymes catalyzing the steps in the biosynthetic pathway are AAE, OLS, OAC, and PT. The present disclosure provides AAE enzymes from source organisms other than C. sativa capable of acting in such a cannabinoid pathway in a recombinant host cell.

FIG. 4 depicts three exemplary two step pathways for converting the rare cannabinoid, CBGVA, to one or more of the rare cannabinoids, Δ⁹-THCVA, CBDVA, and/or CBCVA, and then, optionally, further converting them to the decarboxylated cannabinoids, Δ⁹-THCV, CBDV, and/or CBCV. The first conversion from CBGVA to Δ⁹-THCVA, CBDVA, and/or CBCVA can be catalyzed by a single cannabinoid synthase, CBDAs, THCAs and/or CBCAs, respectively. As described elsewhere herein, in some embodiments the single cannabinoid synthase (e.g., CBDAs) is capable of catalyzing not only the conversion of CBGVA to its preferred product (e.g., CBDAs preferentially converts CBGVA to CBDVA), but also converts CBGVA to one or both of the other cannabinoid acid products, typically in lesser amounts.

FIG. 5 depicts the “Plasmid_030” used to transform yeast strain CEN.PK2-1 D with 11 different yeast-optimized candidate AAE genes via homologous recombination. CEN.PK2-1 D has been engineered with a pathway of the enzymes AAE1, OLS, and OAC, and is capable of converting hexanoic acid (HA) to the cannabinoid precursor olivetolic acid (OA). Plasmid_030 contains a three gene cassette comprised of AAE1, OLS, and OAC. Linearized plasmid_030 minus the gene encoding AAE1 together with the synthesized AAE candidate genes for homologous recombination of CEN.PK2-1 D. The newly recombined yeast strains were tested for the presence of the AAE candidate gene using PCR and sequencing and then screened for the ability to convert butyric acid (BA) to divarinic acid (DA) as described in Example 1.

FIGS. 6A and 6B depict plots of in vivo production of divarinic acid (DA) and the varin cannabinoid, CBGVA by engineered S. cerevisiae strains fed 1 mM butyric acid (BA) or EtOH. The strains are derived from CENPK 2-1 D and have been engineered with the enzymes from C. sativa, OLS (SEQ ID NO: 4), OAC (SEQ ID NO: 6), and PT4 (SEQ ID NO: 8), and an AAE enzyme not from C. sativa as described in Example 3. FIG. 6A shows relative production DA by different strains with different AAE enzymes. FIG. 6B shows relative production CBGVA by different strains with different AAE enzymes.

### DETAILED DESCRIPTION

For the descriptions herein and the appended claims, the singular forms “a”, and “an” include plural referents unless the context clearly indicates otherwise. Thus, for example, reference to “a protein” includes more than one protein, and reference to “a compound” refers to more than one compound. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. The use of “comprise,” “comprises,” “comprising” “include,” “includes,” and “including” are interchangeable and not intended to be limiting. It is to be further understood that where descriptions of various embodiments use the term “comprising,” those skilled in the art would understand that in some specific instances, an embodiment can be alternatively described using language “consisting essentially of” or “consisting of.”

Where a range of values is provided, unless the context clearly dictates otherwise, it is understood that each intervening integer of the value, and each tenth of each intervening integer of the value, unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of these limits, ranges excluding (i) either or (ii) both of those included limits are also included in the invention. For example, “1 to 50,” includes “2 to 25,” “5 to 20,” “25 to 50,” “1 to 10,” etc.

Generally, the nomenclature used herein and the techniques and procedures described herein include those that are well understood and commonly employed by those of ordinary skill in the art, such as the common techniques and methodologies described in e.g., Green and Sambrook, Molecular Cloning: A Laboratory Manual (Fourth Edition), Vols. 1-3, Cold Spring Harbor Laboratory, Cold Spring Harbor, N.Y., 2012 (hereinafter “Sambrook”); and Current Protocols in Molecular Biology, F. M. Ausubel et al., eds., originally published in 1987 in book form by Greene Publishing Associates, Inc. and John Wiley & Sons, Inc., and regularly supplemented through 2011, and now available in journal format online as Current Protocols in Molecular Biology, Vols. 00-130, (1987-2020), published by Wiley & Sons, Inc. in the Wiley Online Library (hereinafter “Ausubel”).

All publications, patents, patent applications, and other documents referenced in this disclosure are hereby incorporated by reference in their entireties for all purposes to the same extent as if each individual publication, patent, patent application or other document were individually indicated to be incorporated by reference herein for all purposes.

Unless defined otherwise, all technical and scientific terms used herein have the same meaning as commonly understood by one of ordinary skill in the art to which the present invention pertains. It is to be understood that the terminology used herein is for describing particular embodiments only and is not intended to be limiting. For purposes of interpreting this disclosure, the following description of terms will apply and, where appropriate, a term used in the singular form will also include the plural form and vice versa.

### Definitions

“Cannabinoid” refers to a compound that acts on cannabinoid receptor, and is intended to include the endocannabinoid compounds that are produced naturally in animals, the phytocannabinoid compounds produced naturally in cannabis plants, and the synthetic cannabinoids compounds. Exemplary cannabinoids of the present disclosure include those compounds listed in Table 1 (below).

TABLE 1
Exemplary cannabinoid compounds

| | Abbrev. | |
|---|---|---|
| Compound Name | Name | Chemical Structure |
| cannabigerolic acid | CBGA | 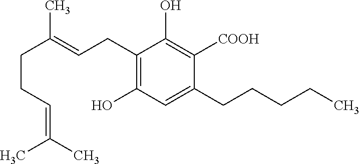 [SMILES: CCCCCc1cc(O)c(C/C=C(\C)CCC=C(C)C)c(O)c1C(=O)O] |
| cannabigerol | CBG | 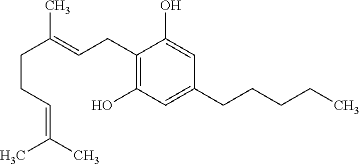 [SMILES: CCCCCc1cc(O)c(C/C=C(\C)CCC=C(C)C)c(O)c1] |
| Δ⁹-tetrahydrocannabinolic acid | Δ⁹-THCA | 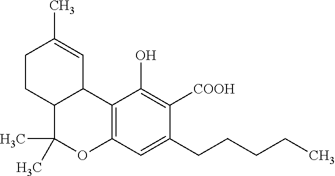 [SMILES: CCCCCc1cc2c(c(O)c1C(=O)O)C1C=C(C)CCC1C(C)(C)O2] |
| Δ⁹-tetrahydrocannabinol | Δ⁹-THC | 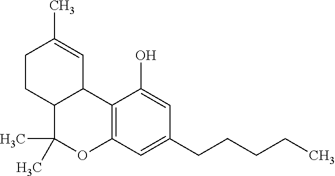 [SMILES: CCCCCc1cc(O)c2c(c1)OC(C)(C)C1CCC(C)=CC21] |
| Δ⁸-tetrahydrocannabinolic acid | Δ⁸-THCA | 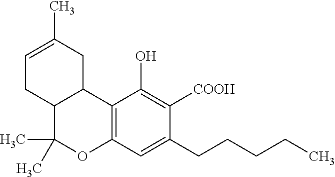 [SMILES: CCCCCc1cc2c(c(O)c1C(=O)O)C1CC(C)=CCC1C(C)(C)O2] |
| Δ⁸-tetrahydrocannabinol | Δ⁸-THC | 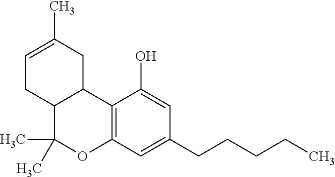 [SMILES: CCCCCc1cc(O)c2c(c1)OC(C)(C)C1CC=C(C)CC21] |
| cannabidiolic acid | CBDA | 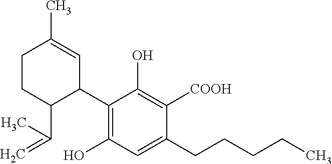 [SMILES: C=C(C)C1CCC(C)=CC1c1c(O)cc(CCCCC)c(C(=O)O)c1O] |
| cannabidiol | CBD | 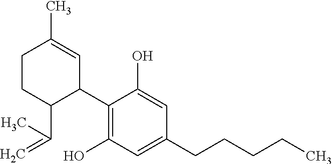 [SMILES: C=C(C)C1CCC(C)=CC1c1c(O)cc(CCCCC)cc1O] |
| cannabichromenic acid | CBCA | 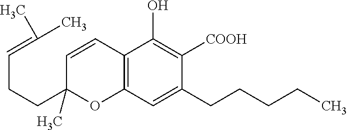 [SMILES: CCCCCc1cc2c(c(O)c1C(=O)O)C=CC(C)(CCC=C(C)C)O2] |
| cannabichromene | CBC | 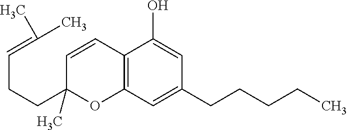 [SMILES: CCCCCc1cc(O)c2c(c1)OC(C)(CCC=C(C)C)C=C2] |
| cannabinolic acid | CBNA | 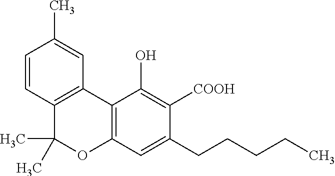 [SMILES: CCCCCc1cc2c(c(O)c1C(=O)O)-c1cc(C)ccc1C(C)(C)O2] |
| cannabinol | CBN | 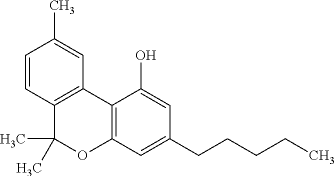 [SMILES: CCCCCc1cc(O)c2c(c1)OC(C)(C)c1ccc(C)cc1-2] |
| cannabidivarinic acid | CBDVA | 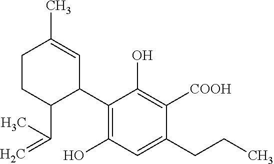 [SMILES: C=C(C)C1CCC(C)=CC1c1c(O)cc(CCC)c(C(=O)O)c1O] |
| cannabidivarin | CBDV | 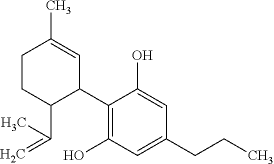 [SMILES: C=C(C)C1CCC(C)=CC1c1c(O)cc(CCC)cc1O] |
| Δ⁹-tetrahydrocannabivarinic acid | Δ⁹-THCVA | 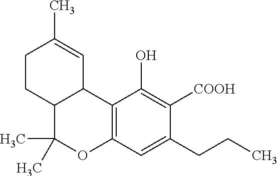 [SMILES: CCCc1cc2c(c(O)c1C(=O)O)C1C=C(C)CCC1C(C)(C)O2] |
| Δ⁹-tetrahydrocannabivarin | Δ⁹-THCV | 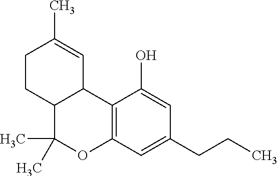 [SMILES: CCCc1cc(O)c2c(c1)OC(C)(C)C1CCC(C)=CC21] |
| Cannabidibutolic acid | CBDBA | 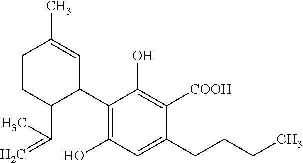 [SMILES: C=C(C)C1CCC(C)=CC1c1c(O)cc(CCCC)c(C(=O)O)c1O] |
| Cannabidibutol | CBDB | 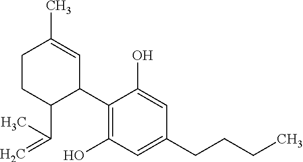 [SMILES: C=C(C)C1CCC(C)=CC1c1c(O)cc(CCCC)cc1O] |
| Δ⁹-tetrahydrocannabutolic acid | Δ⁹-THCBA | 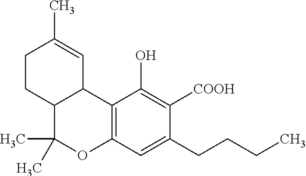 [SMILES: CCCCc1cc2c(c(O)c1C(=O)O)C1C=C(C)CCC1C(C)(C)O2] |
| Δ⁹-tetrahydrocannabutol | Δ⁹-THCB | 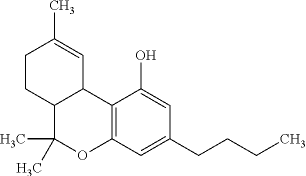 [SMILES: CCCCc1cc(O)c2c(c1)OC(C)(C)C1CCC(C)=CC21] |
| Cannabidiphorolic acid | CBDPA | 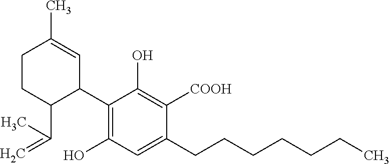 [SMILES: C=C(C)C1CCC(C)=CC1c1c(O)cc(CCCCCCC)c(C(=O)O)c1O] |
| Cannabidiphorol | CBDP | 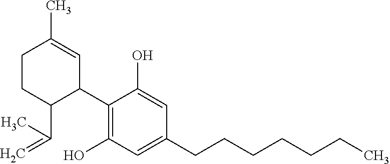 [SMILES: C=C(C)C1CCC(C)=CC1c1c(O)cc(CCCCCCC)cc1O] |
| Δ⁹-tetrahydrocannabiphorolic acid | Δ⁹-THCPA | 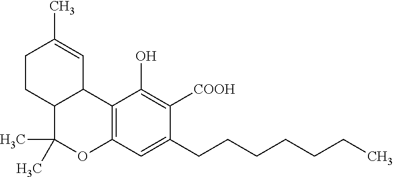 [SMILES: CCCCCCCc1cc2c(c(O)c1C(=O)O)C1C=C(C)CCC1C(C)(C)O2] |
| Δ⁹-tetrahydrocannabiphorol | Δ⁹-THCP | 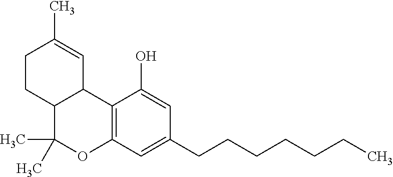 [SMILES: CCCCCCCc1cc(O)c2c(c1)OC(C)(C)C1CCC(C)=CC21] |
| cannabichromevarinic acid | CBCVA | 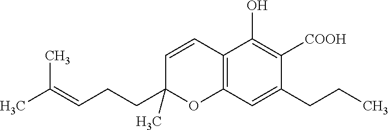 [SMILES: CCCc1cc2c(c(O)c1C(=O)O)C=CC(C)(CCC=C(C)C)O2] |
| cannabichromevarin | CBCV | 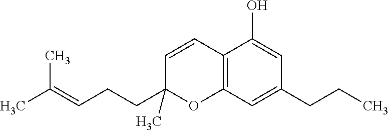 [SMILES: CCCc1cc(O)c2c(c1)OC(C)(CCC=C(C)C)C=C2] |
| cannabigerovarinic acid | CBGVA | 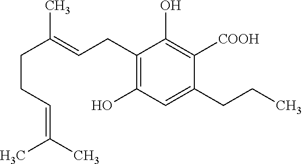 [SMILES: CCCc1cc(O)c(C/C=C(\C)CCC=C(C)C)c(O)c1C(=O)O] |
| cannabigerovarin | CBGV | 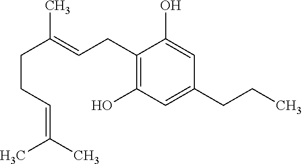 [SMILES: CCCc1cc(O)c(C/C=C(\C)CCC=C(C)C)c(O)c1] |
| cannabicyclolic acid | CBLA | 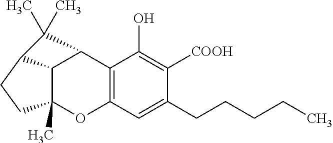 |
| cannabicyclol | CBL | 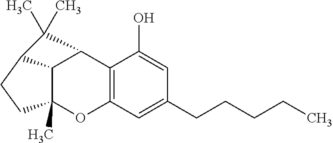 |
| cannabielsoinic acid | CBEA | 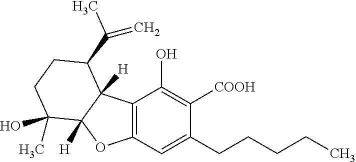 [SMILES: C=C(C)[C@@H]1CC[C@](C)(O)[C@H]2Oc3cc(CCCCC)c(C(=O)O)c(O)c3[C@@H]12] |
| cannabielsoin | CBE | 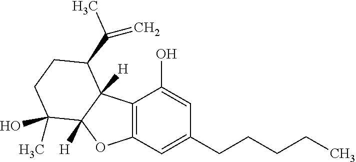 [SMILES: C=C(C)[C@@H]1CC[C@](C)(O)[C@H]2Oc3cc(CCCCC)cc(O)c3[C@@H]12] |
| cannabicitranic acid | CBTA | 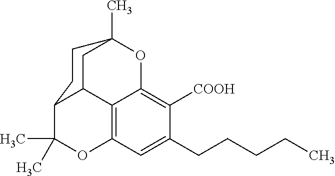 [SMILES: CCCCCc1cc2c3c(c1C(=O)O)OC1(C)CCC(C3C1)C(C)(C)O2] |
| cannabicitran | CBT | 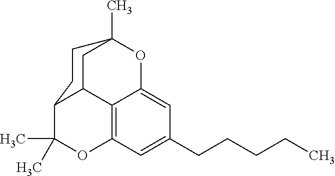 [SMILES: CCCCCc1cc2c3c(c1)OC(C)(C)C1CCC(C)(CC31)O2] |

“Pathway” refers an ordered sequence of enzymes that act in a linked series to convert an initial substrate molecule into final product molecule. As used herein, “pathway” is intended to encompass naturally-occurring pathways and non-naturally occurring, recombinant pathways. Accordingly, a pathway of the present disclosure can include a series of enzymes that are naturally-occurring and/or non-naturally occurring, and can include a series of enzymes that act in vivo or in vitro.

“Pathway capable of producing a cannabinoid” or “cannabinoid pathway” refers to a pathway that can convert an cannabinoid precursor molecule, such as hexanoic acid, into a final product molecule that is a cannabinoid, such as cannabigerolic acid (CBGA). For example, the four enzymes AAE, OLS, OAC, and PT4 which convert hexanoic acid to CBGA, form a pathway capable of producing a cannabinoid.

Cannabinoid precursor” as used herein refers to a compound capable of being converted into a cannabinoid by a pathway capable producing a cannabinoid. Cannabinoid precursors as referenced in the present disclosure include, but are not limited to, the exemplary naturally occurring and synthetic cannabinoid precursors with varying alkyl carbon chain lengths summarized in Table 2 (below).

TABLE 2
Exemplary cannabinoid precursor compounds

| | Abbrev. | |
|---|---|---|
| Compound Name | Name | Chemical Structure |
| Orcinolic acid (2,4-dihydroxy-6- methylbenzoic acid) | | 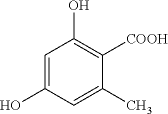 |
| Divarinic acid (2,4-dihydroxy-6- propylbenzoic acid) | DA | 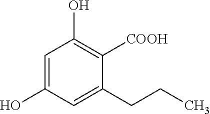 |
| Butolic acid (2-butyl-4,6- dihydroxybenzoic acid) | BA | 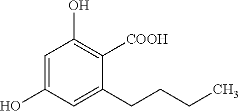 |
| Olivetolic acid (2,4-dihydroxy-6- pentylbenzoic acid) | OA | 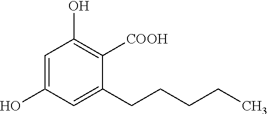 [SMILES: CCCCCc1cc(O)cc(O)c1C(=O)O] |
| 2-hexyl-4,6- dihydroxybenzoic acid | DHBA | 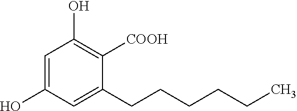 [SMILES: CCCCCCc1cc(O)cc(O)c1C(=O)O] |
| Sphaerophorolic acid (2-heptyl-4,6- dihydroxybenzoic acid) | PA | 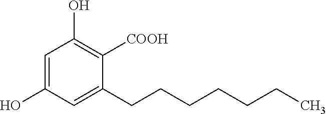 [SMILES: CCCCCCCc1cc(O)cc(O)c1C(=O)O] |

“Conversion” as used herein refers to the enzymatic conversion of the substrate(s) to the corresponding product(s). “Percent conversion” refers to the percent of the substrate that is converted to the product within a period of time under specified conditions. Thus, the “enzymatic activity” or “activity” of an enzymatic conversion can be expressed as “percent conversion” of the substrate to the product.

“Substrate” as used herein in the context of an enzyme mediated process refers to the compound or molecule acted on by the enzyme.

“Product” as used herein in the context of an enzyme mediated process refers to the compound or molecule resulting from the activity of the enzyme.

“Host cell” as used herein refers to a cell capable of being functionally modified with recombinant nucleic acids and functioning to express recombinant products, including polypeptides and compounds produced by activity of the polypeptides.

“Nucleic acid,” or “polynucleotide” as used herein interchangeably to refer to two or more nucleosides that are covalently linked together. The nucleic acid may be wholly comprised ribonucleosides (e.g., RNA), wholly comprised of 2′-deoxyribonucleotides (e.g., DNA) or mixtures of ribo- and 2′-deoxyribonucleosides. The nucleoside units of the nucleic acid can be linked together via phosphodiester linkages (e.g., as in naturally occurring nucleic acids), or the nucleic acid can include one or more non-natural linkages (e.g., phosphorothioester linkage). Nucleic acid or polynucleotide is intended to include single-stranded or double-stranded molecules, or molecules having both single-stranded regions and double-stranded regions. Nucleic acid or polynucleotide is intended to include molecules composed of the naturally occurring nucleobases (i.e., adenine, guanine, uracil, thymine and cytosine), or molecules comprising that include one or more modified and/or synthetic nucleobases, such as, for example, inosine, xanthine, hypoxanthine, etc.

“Protein,” “polypeptide,” and “peptide” are used herein interchangeably to denote a polymer of at least two amino acids covalently linked by an amide bond, regardless of length or post-translational modification (e.g., glycosylation, phosphorylation, lipidation, myristilation, ubiquitination, etc.). As used herein “protein” or “polypeptide” or “peptide” polymer can include D- and L-amino acids, and mixtures of D- and L-amino acids.

“Naturally-occurring” or “wild-type” as used herein refers to the form as found in nature. For example, a naturally occurring nucleic acid sequence is the sequence present in an organism that can be isolated from a source in nature and which has not been intentionally modified by human manipulation.

“Recombinant,” “engineered,” or “non-naturally occurring” when used herein with reference to, e.g., a cell, nucleic acid, or polypeptide, refers to a material, or a material corresponding to the natural or native form of the material, that has been modified in a manner that would not otherwise exist in nature, or is identical thereto but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Nucleic acid derived from” as used herein refers to a nucleic acid having a sequence at least substantially identical to a sequence of found in naturally in an organism. For example, cDNA molecules prepared by reverse transcription of mRNA isolated from an organism, or nucleic acid molecules prepared synthetically to have a sequence at least substantially identical to, or which hybridizes to a sequence at least substantially identical to a nucleic sequence found in an organism.

“Coding sequence” refers to that portion of a nucleic acid (e.g., a gene) that encodes an amino acid sequence of a protein.

“Heterologous nucleic acid” as used herein refers to any polynucleotide that is introduced into a host cell by laboratory techniques, and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the imine reductase enzymes may be codon optimized for optimal production from the host organism selected for expression.

“Preferred, optimal, high codon usage bias codons” refers to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (see GCG CodonPreference, Genetics Computer Group Wisconsin Package; CodonW, John Peden, University of Nottingham; McInerney, J. O, 1998, Bioinformatics 14:372-73; Stenico et al., 1994, Nucleic Acids Res. 222437-46; Wright, F., 1990, Gene 87:23-29). Codon usage tables are available for a growing list of organisms (see for example, Wada et al., 1992, Nucleic Acids Res. 20:2111-2118; Nakamura et al., 2000, Nucl. Acids Res. 28:292; Duret, et al., supra; Henaut and Danchin, “Escherichia coli and Salmonella,” 1996, Neidhardt, et al. Eds., ASM Press, Washington D.C., p. 2047-2066. The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (see for example, Mount, D., Bioinformatics: Sequence and Genome Analysis, Chapter 8, Cold Spring Harbor Laboratory Press, Cold Spring Harbor, N.Y., 2001; Uberbacher, E. C., 1996, Methods Enzymol. 266:259-281; Tiwari et al., 1997, Comput. Appl. Biosci. 13:263-270).

“Control sequence” as used herein refers to all sequences, which are necessary or advantageous for the expression of a polynucleotide and/or polypeptide as used in the present disclosure. Each control sequence may be native or foreign to the nucleic acid sequence encoding a polypeptide. Such control sequences include, but are not limited to, a leader, a promoter, a polyadenylation sequence, a pro-peptide sequence, a signal peptide sequence, and a transcription terminator. At a minimum, control sequences typically include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” as used herein refers to a configuration in which a control sequence is appropriately placed (e.g., in a functional relationship) at a position relative to a polynucleotide sequence or polypeptide sequence of interest such that the control sequence directs or regulates the expression of the sequence of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Percentage of sequence identity,” “percent sequence identity,” “percentage homology,” or “percent homology” are used interchangeably herein to refer to values quantifying comparisons of the sequences of polynucleotides or polypeptides, and are determined by comparing two optimally aligned sequences over a comparison window, wherein the portion of the polynucleotide or polypeptide sequence in the comparison window may comprise additions or deletions (or gaps) as compared to the reference sequence for optimal alignment of the two sequences. The percentage values may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman, 1981, Adv. Appl. Math. 2:482, by the homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Software Package), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)). Examples of algorithms that are suitable for determining percent sequence identity and sequence similarity are the BLAST and BLAST 2.0 algorithms, which are described in Altschul et al., 1990, J. Mol. Biol. 215: 403-410 and Altschul et al., 1977, Nucleic Acids Res. 3389-3402, respectively. Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff and Henikoff, 1989, Proc Natl Acad Sci USA 89:10915). Exemplary determination of sequence alignment and % sequence identity can employ the BESTFIT or GAP programs in the GCG Wisconsin Software package (Accelrys, Madison Wis.), using default parameters provided.

“Reference sequence” refers to a defined sequence used as a basis for a sequence comparison. A reference sequence may be a subset of a larger sequence, for example, a segment of a full-length nucleic acid or polypeptide sequence. A reference sequence typically is at least 20 nucleotide or amino acid residue units in length, but can also be the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. “Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (or gaps) of 20 percent or less as compared to the reference sequence (which does not comprise additions or deletions) for optimal alignment of the two sequences.

“Substantial identity” or “substantially identical” refers to a polynucleotide or polypeptide sequence that has at least 70% sequence identity, at least 80% sequence identity, at least 85% sequence identity, at least 90% sequence identity, at least 95% sequence identity, or at least 99% sequence identity, as compared to a reference sequence over a comparison window of at least 20 nucleoside or amino acid residue positions, frequently over a window of at least 30-50 positions, wherein the percentage of sequence identity is calculated by comparing the reference sequence to a sequence that includes deletions or additions which total 20 percent or less of the reference sequence over the window of comparison.

“Corresponding to,” “reference to,” or “relative to” when used in the context of the numbering of a given amino acid or polynucleotide sequence refers to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered imine reductase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Isolated” as used herein in reference to a molecule means that the molecule (e.g., cannabinoid, polynucleotide, polypeptide) is substantially separated from other compounds that naturally accompany it, e.g., protein, lipids, and polynucleotides. The term embraces nucleic acids which have been removed or purified from their naturally-occurring environment or expression system (e.g., host cell or in vitro synthesis).

“Substantially pure” refers to a composition in which a desired molecule is the predominant species present (i.e., on a molar or weight basis it is more abundant than any other individual macromolecular species in the composition), and is generally a substantially purified composition when the object species comprises at least about 50 percent of the macromolecular species present by mole or % weight.

“Recovered” as used herein in relation to an enzyme, protein, or cannabinoid compound, refers to a more or less pure form of the enzyme, protein, or cannabinoid.

Recombinant Host Cells for Production of Cannabinoids or Cannabinoid Precursors Using an AAE Enzyme not from Cannabis sativa

The present disclosure provides recombinant host cells (e.g., S. cerevisiae) that comprise a functional pathway capable of enhanced production of a cannabinoid precursor (e.g., olivetolic acid or divarinic acid) and/or cannabinoid (e.g., CBGA or CBGVA), and/or a cannabinoid, where the pathway includes the enzymes AAE, OAC, OLS, and optionally, PT4, and the AAE enzyme is not the AAE enzyme AAE1 from Cannabis sativa having the amino acid sequence of SEQ ID NO: 2. The AAE1 polypeptide in the cannabinoid pathway of C. sativa has coenzyme A synthetase activity that produces the activated thioester, hexanoyl-CoA (compound (1)) from the hexanoic acid (HA) substrate(compound (2)), as shown in Scheme 1.

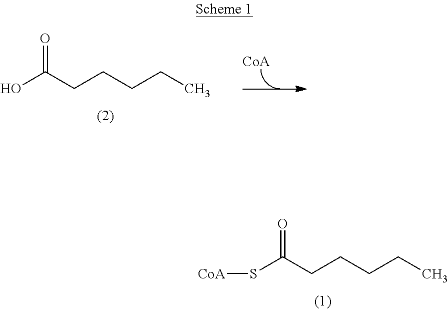

AAE1 has been shown to have some CoA synthetase activity with linear alkanoic acid substrates of varying lengths, including butyric acid (4), which it acts upon to produce the varin cannabinoid precursor, butyroyl-CoA (3), as shown in Scheme 2.

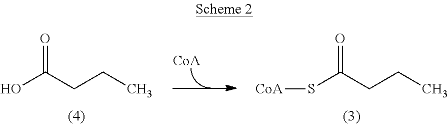

The present disclosure provides recombinant host cells with a cannabinoid pathway comprising an enzyme with AAE activity derived from a plant source organism other than C. sativa, such as a plant source selected from Amentotaxus argotaenia; Callitris macleayana; Cephalotaxus harringtonia; Diselma archeri; Humulus lupulus; Prumnopitys andina; Taxus x media; and Widdringtonia cedarbergensis. The amino acid sequences of the AAE enzymes from these plant sources differ substantially from the sequence of C. sativa AAE1, for example, having an amino acid sequence of less than 60% identity to SEQ ID NO: 2. It is a surprising technical effect of the present disclosure that these AAE enzymes not from Cannabis plants when incorporated in a cannabinoid pathway in a recombinant host system can result in production of cannabinoids (such as CBGA, CBGVA) and cannabinoid precursors (such as OA, DA). In some cases, the production of the cannabinoids and/or cannabinoid precursors is enhanced relative to a control host cell that comprises the same pathway of enzymes with AAE1 of C. sativa of SEQ ID NO: 2 as the AAE enzyme.

Exemplary AAE enzymes from these plant sources and their nucleotide and amino acid sequences are disclosed below in Table 3, the accompanying Sequence Listing, and further described in the Examples.

TABLE 3
AAE Enzymes for Recombinant Cannabinoid
Precursor and Cannabinoid Biosynthesis

| | | SEQ ID | SEQ ID |
|---|---|---|---|
| AAE | | NO: | NO: |
| Abbrev. | Source Organism | (nt) | (aa) |
| TM4 | Taxus x media | 15 | 16 |
| CCL2 | Humulus lupulus | 17 | 18 |
| CM1 | Callitris macleayana | 19 | 20 |
| DA1 | Diselma archeri | 21 | 22 |
| CCL3 | Humulus lupulus | 23 | 24 |
| AA1 | Amentotaxus argotaenia | 25 | 26 |
| WC1 | Widdringtonia cedarbergensis | 27 | 28 |
| CH3 | Cephalotaxus harringtonia | 29 | 30 |
| CH2 | Cephalotaxus harringtonia | 31 | 32 |
| PA1 | Prumnopitys andina | 33 | 34 |
| TMS | Taxus x media | 35 | 36 |
| MT1 | Microcachrys tetragona | 37 | 38 |
| AC1 | Athrotaxis cupressoides | 39 | 40 |
| LS1 | Larix speciosa | 41 | 42 |
| AS1 | Austrotaxus spicata | 43 | 44 |
| HB1 | Halocarpus bidwillii | 45 | 46 |
| TC1 | Taiwania cryptomerioides | 47 | 48 |
| DC1 | Dacrycarpus compactus | 49 | 50 |
| CMI2 | Cinnamomum micranthum | 51 | 52 |
| | f. kanehirae | | |
| CD1 | Calocedrus decurrens | 53 | 54 |
| PR1 | Podocarpus rubens | 55 | 56 |
| PC1 | Pseudotaxus chienii | 57 | 58 |
| TM15 | Taxus x media | 59 | 60 |
| TS1 | Tetraclinis sp. | 61 | 62 |
| NN2 | Nageia nagi | 63 | 64 |
| OB2 | Oncotheca balansae | 65 | 66 |
| GP1 | Glyptostrobus pensilis | 67 | 68 |
| PE1 | Picea engelmannii | 69 | 70 |
| CDU1 | Cupressus dupreziana | 71 | 72 |
| AA2 | Amentotaxus argotaenia | 73 | 74 |
| PA2 | Prumnopitys andina | 75 | 76 |
| AL1 | Abies lasiocarpa | 77 | 78 |
| CH1 | Cephalotaxus harringtonia | 79 | 80 |
| CLA1 | Chamaecyparis lawsoniana | 81 | 82 |
| CL1 | Cunninghamia lanceolate | 83 | 84 |
| | (branch apex with needles) | | |
| NN1 | Nageia nagi | 85 | 86 |
| DE1 | Dioon edule | 87 | 88 |
| FH1 | Fokienia hodginsii | 89 | 90 |
| CJ1 | Cryptomeria japonica | 91 | 92 |
| DB1 | Dacrydium balansae | 93 | 94 |
| OB1 | Oncotheca balansae | 95 | 96 |
| TM6 | Taxus x media | 97 | 98 |

It is contemplated that

The surprising technical effect of enhanced biosynthesis of the cannabinoid associated with the introduction of an AAE enzyme from a plant source other than C. sativa into a heterologous cannabinoid pathway comprising OAC, OLS, (and, optionally, PT4), provides a distinct and unexpected advantage of these recombinant host cells for use in the production of the cannabinoids, including the rare varin cannabinoid, CBGVA.

Additionally, the recombinant host cells described herein are capable of producing the cannabinoid precursor compounds: (a) olivetolic acid (also referred to herein as “OA”), when cultured in the presence the feedstock compound, hexanoic acid (also referred to herein as “HA”); and/or (b) divarinic acid (also referred to herein as “DA” or “divaric acid”) when cultured in the presence the feedstock compound, butyric acid (also referred to herein as “BA”). The ability to use HA and/or BA as the feedstock for fermentative production of the cannabinoid precursor compounds, OA and/or DA provides another significant advantage for the use of host cells in cannabinoid biosynthesis.

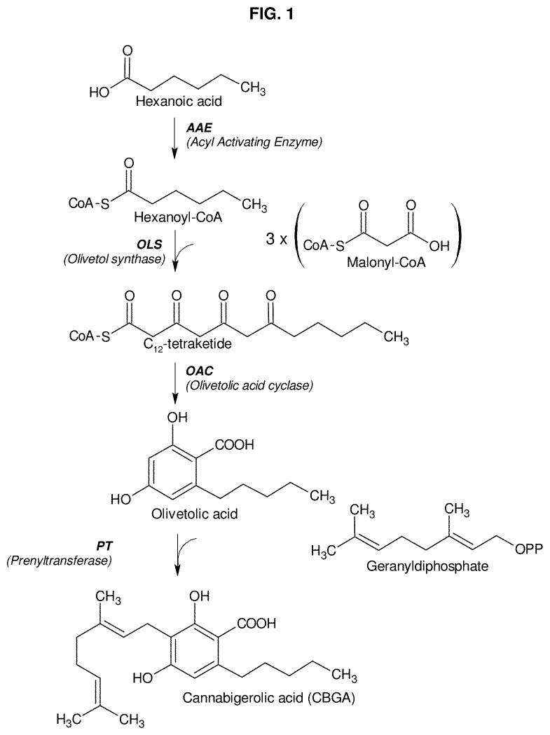

An exemplary cannabinoid pathway capable of converting hexanoic acid (HA) to cannabinoid precursor olivetolic acid (OA) and further converting the OA to the cannabinoid, CBGA is depicted in FIG. 1, where the conversion of HA to OA is carried out by the sequence of the enzymes, Acyl Activating Enzyme (AAE), Olivetol Synthase (OLS), and Olivetolic Acid Cyclase (OAC). Accordingly, in at least one embodiment of the present disclosure, the methods and compositions for converting HA to OA use a recombinant host cell that comprises a heterologous cannabinoid pathway of at least the three enzymes, AAE, OLS, and OAC, wherein the AAE is from plant source other than C. sativa. As further illustrated in FIG. 1, the heterologous pathway can also comprise enzymes capable of catalyzing the further downstream conversion of OA to CBGA. The addition of a prenyltransferase enzyme (e.g., PT4) to the heterologous pathway comprising AAE, OAC, and OLS, allows for the further conversion of OA to the cannabinoid, CBGA. Thus, one of the further surprising advantages of the present disclosure is that the use of an AAE from a plant source other than C. sativa allows for the conversion of an HA feedstock substrate into not only the cannabinoid precursor compound, OA, but also the cannabinoid, CBGA, as shown in FIG. 1.

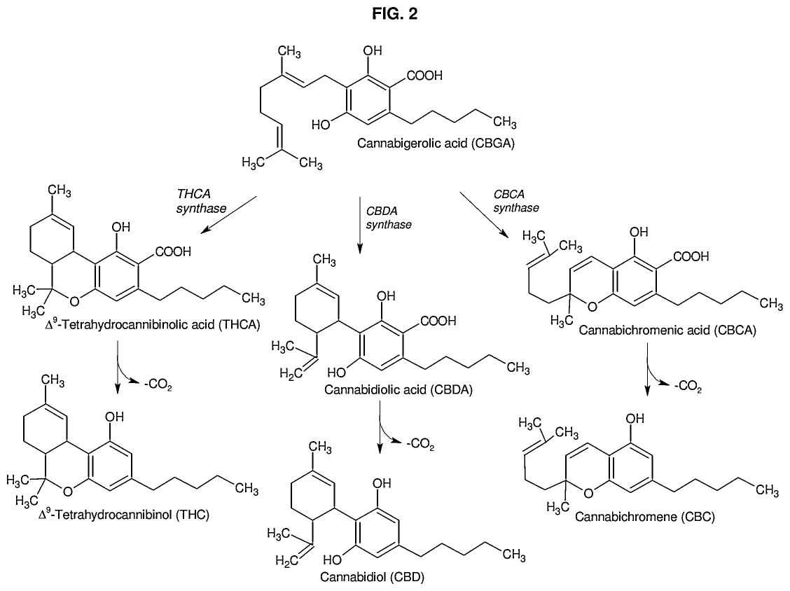

The heterologous pathway depicted in FIG. 1 which is capable of producing a cannabinoid, such as CBGA, can be further modified to include one or more cannabinoid synthase enzymes (e.g., CBDAS, THCAS, CBCAS). As shown by the exemplary pathway of FIG. 2, with the incorporation of one or more synthase enzymes, the cannabinoid, CBGA, can be converted to the downstream cannabinoids, cannabidiolic acid (CBDA), Δ⁹-tetrahydrocannabinolic acid (Δ⁹-THCA), and cannabichromenic acid (CBCA). Enzymes capable of carrying out these conversions include the synthases from C. sativa, CBDA synthase (CBDAS), THCA synthase (THCAS), and CBCA synthase (CBCAS), respectively. Furthermore, as shown in FIG. 2, the cannabinoids, CBDA, Δ⁹-THCA, and CBCA, can undergo a further decarboxylation reaction to provide the cannabinoid products, cannabidiol (CBD), Δ⁹-tetrahydrocannabinol (Δ⁹-THC), and cannabichromene (CBC), respectively. In some embodiments, this further decarboxylation can be carried out under in vitro reaction conditions using the cannabinoid acids (i.e., CBDA, THCA, and CBCA) isolated from the recombinant host cells.

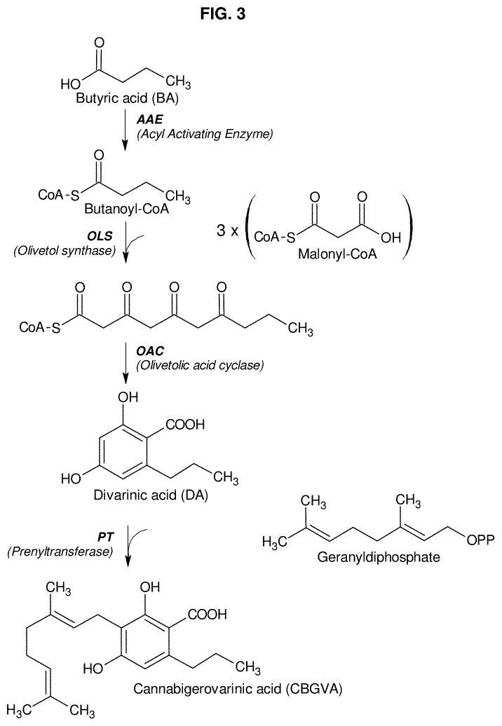

Although FIG. 1 illustrates the cannabinoid pathway of AAE, OLS, and OAC as carrying out the production of the cannabinoid precursor compound, OA and/or the cannabinoid CBGA, from HA feedstock, this same pathway is also capable of producing the rare cannabinoid precursor compound, divarinic acid (DA), and the rare varin cannabinoid, CBGVA, from butyric acid (BA) feedstock. An exemplary cannabinoid pathway capable of converting BA to DA and further converting the DA to CBGVA is depicted in FIG. 3, where the conversion of BA to DA is carried out by the sequence of the enzymes, Acyl Activating Enzyme (AAE), Olivetol Synthase (OLS), and Olivetolic Acid Cyclase (OAC). Accordingly, in at least one embodiment of the present disclosure, the methods and compositions for converting butyric acid (BA) to divarinic acid (DA) use a recombinant host cell that comprises a heterologous pathway comprises at least the three enzymes, AAE, OLS, and OAC, wherein the AAE is from plant source other than C. sativa. As shown in FIG. 3, the heterologous pathway can also comprise enzymes capable of catalyzing the further downstream conversion of divarinic acid (DA) to cannabigerovarinic acid (CBGVA). As noted above, the addition of a prenyltransferase enzyme (e.g., PT4) to the heterologous pathway comprising AAE, OAC, and OLS, allows for the further conversion of DA into a rare cannabinoid compound, such as the varin cannabinoid, cannabigerovarinic acid (CBGVA). Thus, one further advantage of the present disclosure is that the use of an AAE from a plant source other than C. sativa allows for the conversion of BA feedstock substrate into not only the rare cannabinoid precursor compound, DA, but also the rare cannabinoid, CBGVA, as shown in FIG. 3.

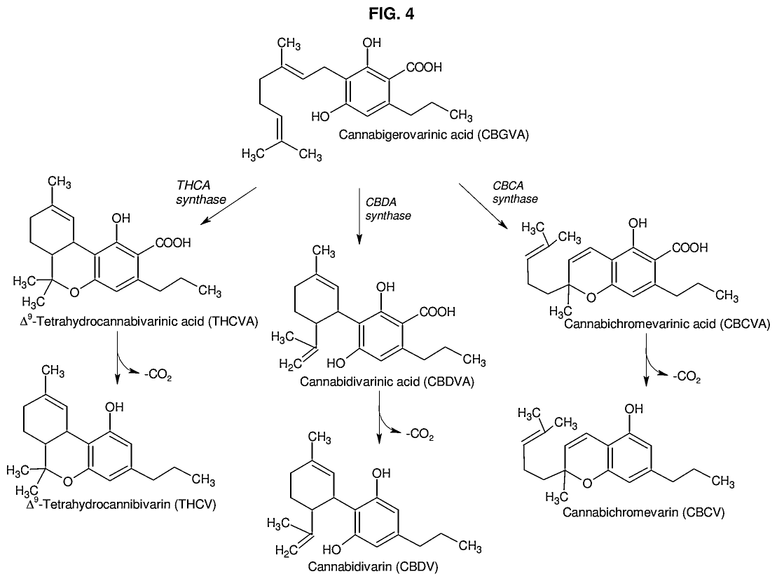

The heterologous pathway depicted in FIG. 3 which is capable of producing a rare cannabinoid, such as CBGVA, can be further modified to include one or more cannabinoid synthase enzymes (e.g., CBDAS, THCAS, CBCAS). As shown by the exemplary pathway of FIG. 4, with the incorporation of one or more synthase enzymes, the rare varin cannabinoid, CBGVA, can be converted to the rare varin cannabinoids, cannabidivarinic acid (CBDVA), Δ⁹-tetrahydrocannabivarinic acid (Δ⁹-THCVA), and cannabichromevarinic acid (CBCVA). Enzymes capable of carrying out these conversions include the C. sativa CBDA synthase, THCA synthase, and CBCA synthase, respectively. Furthermore, as shown in FIG. 4, the rare cannabinoids, CBDVA, Δ⁹-THCVA, and CBCVA, can undergo a further decarboxylation reaction to provide the varin cannabinoid products, cannabidivarin (CBDV), Δ⁹-tetrahydrocannabivarin (Δ⁹-THCV), and cannabichromevarin (CBCV), respectively. In some embodiments, this further decarboxylation can be carried out under in vitro reaction conditions using the cannabinoid acids isolated from the recombinant host cells.

Cannabinoid pathway enzymes that can be introduced into a recombinant host cell to provide the pathways illustrated in FIGS. 1, 2, 3 and 4 include, but are not limited to, the cannabinoid pathway enzymes from Cannabis sativa, OLS, OAC, PT4, and/or CBDAS, as described in Table 4 (below).

TABLE 4
Exemplary cannabinoid pathway enzymes

| | | SEQ | SEQ |
|---|---|---|---|
| | | ID | ID |
| Enzyme Name | | NO: | NO: |
| (abbreviation) | Polypeptide Sequence | (nt) | (aa) |
| Acyl activating | MGKNYKSLDSVVASDFIALGITSEVAETLHGRLAEIVCNYGA | 1 | 2 |
| enzyme | ATPQTWINIANHILSPDLPFSLHQMLFYGCYKDFGPAPPAWI | | |
| (AAE1) | PDPEKVKSTNLGALLEKRGKEFLGVKYKDPISSFSHFQEFSV | | |
| [Cannabis | RNPEVYWRTVLMDEMKISFSKDPECILRRDDINNPGGSEWLP | | |
| sativa] | GGYLNSAKNCLNVNSNKKLNDTMIVWRDEGNDDLPLNKLTLD | | |
| AFD33345.1 | QLRKRVWLVGYALEEMGLEKGCAIAIDMPMHVDAVVIYLAIV | | |
| | LAGYVVVSIADSFSAPEISTRLRLSKAKAIFTQDHIIRGKKR | | |
| | IPLYSRVVEAKSPMAIVIPCSGSNIGAELRDGDISWDYFLER | | |
| | AKEFKNCEFTAREQPVDAYTNILFSSGTTGEPKAIPWTQATP | | |
| | LKAAADGWSHLDIRKGDVIVWPTNLGWMMGPWLVYASLLNGA | | |
| | SIALYNGSPLVSGFAKFVQDAKVTMLGVVPSIVRSWKSTNCV | | |
| | SGYDWSTIRCFSSSGEASNVDEYLWLMGRANYKPVIEMCGGT | | |
| | EIGGAFSAGSFLQAQSLSSFSSQCMGCTLYILDKNGYPMPKN | | |
| | KPGIGELALGPVMFGASKTLLNGNHHDVYFKGMPTLNGEVLR | | |
| | RHGDIFELTSNGYYHAHGRADDTMNIGGIKISSIEIERVCNE | | |
| | VDDRVFETTAIGVPPLGGGPEQLVIFFVLKDSNDTTIDLNQL | | |
| | RLSFNLGLQKKLNPLFKVTRVVPLSSLPRTATNKIMRRVLRQ | | |
| | QFSHFE | | |
| Olivetol | MNHLRAEGPASVLAIGTANPENILLQDEFPDYYFRVTKSEHM | 3 | 4 |
| synthase | TQLKEKFRKICDKSMIRKRNCFLNEEHLKQNPRLVEHEMQTL | | |
| (OLS) | DARQDMLVVEVPKLGKDACAKAIKEWGQPKSKITHLIFTSAS | | |
| [Cannabis | TTDMPGADYHCAKLLGLSPSVKRVMMYQLGCYGGGTVLRIAK | | |
| sativa] | DIAENNKGARVLAVCCDIMACLFRGPSESDLELLVGQAIFGD | | |
| BAG14339.1 | GAAAVIVGAEPDESVGERPIFELVSTGQTILPNSEGTIGGHI | | |
| | REAGLIFDLHKDVPMLISNNIEKCLIEAFTPIGISDWNSIFW | | |
| | ITHPGGKAILDKVEEKLHLKSDKFVDSRHVLSEHGNMSSSTV | | |
| | LFVMDELRKRSLEEGKSTTGDGFEWGVLFGFGPGLTVERVVV | | |
| | RSVPIKY | | |
| Olivetolic acid | MAVKHLIVLKFKDEITEAQKEEFFKTYVNLVNIIPAMKDVYW | 5 | 6 |
| cyclase | GKDVTQKNKEEGYTHIVEVTFESVETIQDYIIHPAHVGFGDV | | |
| (OAC) | YRSFWEKLLIFDYTPRK | | |
| [Cannabis | | | |
| sativa] | | | |
| AFN42527.1 | | | |
| Aromatic | MGLSLVCTFSFQTNYHTLLNPHNKNPKNSLLSYQHPKTPIIK | 7 | 8 |
| prenyl- | SSYDNFPSKYCLTKNFHLLGLNSHNRISSQSRSIRAGSDQIE | | |
| transferase | GSPHHESDNSIATKILNFGHTCWKLQRPYVVKGMISIACGLF | | |
| (P14) | GRELFNNRHLFSWGLMWKAFFALVPILSFNFFAAIMNQIYDV | | |
| [Cannabis | DIDRINKPDLPLVSGEMSIETAWILSIIVALTGLIVTIKLKS | | |
| sativa] | APLFVFIYIFGIFAGFAYSVPPIRWKQYPFTNFLITISSHVG | | |
| DAC76710.1 | LAFTSYSATTSALGLPFVWRPAFSFIIAFMTVMGMTIAFAKD | | |
| | ISDIEGDAKYGVSTVATKLGARNMTFVVSGVLLLNYLVSISI | | |
| | GIIWPQVFKSNIMILSHAILAFCLIFQTRELALANYASAPSR | | |
| | QFFEFIWLLYYAEYFVYVFI | | |
| Aromatic | IEGSPHHESDNSIATKILNFGHTCWKLQRPYVVKGMISIACG | 9 | 10 |
| prenyl- | LFGRELFNNRHLFSWGLMWKAFFALVPILSFNFFAAIMNQIY | | |
| transferase | DVDIDRINKPDLPLVSGEMSIETAWILSIIVALTGLIVTIKL | | |
| (d82_PT4) | KSAPLFVFIYIFGIFAGFAYSVPPIRWKQYPFTNFLITISSH | | |
| (82 aa N-term | VGLAFTSYSATTSALGLPFVWRPAFSFIIAFMTVMGMTIAFA | | |
| truncation) | KDISDIEGDAKYGVSTVATKLGARNMTFVVSGVLLLNYLVSI | | |
| | SIGIIWPQVFKSNIMILSHAILAFCLIFQTRELALANYASAP | | |
| | SRQFFEFIWLLYYAEYFVYVFI | | |
| CBDA synthase | MKCSTFSFWFVCKIIFFFFSFNIQTSIANPRENFLKCFSQYI | 11 | 12 |
| (CBDAS) | PNNATNLKLVYTQNNPLYMSVLNSTIHNLRFTSDTTPKPLVI | | |
| [Cannabis | VTPSHVSHIQGTILCSKKVGLQIRTRSGGHDSEGMSYISQVP | | |
| sativa] | FVIVDLRNMRSIKIDVHSQTAWVEAGATLGEVYYWVNEKNEN | | |
| BAF65033.1 | LSLAAGYCPTVCAGGHFGGGGYGPLMRNYGLAADNIIDAHLV | | |
| | NVHGKVLDRKSMGEDLFWALRGGGAESFGIIVAWKIRLVAVP | | |
| | KSTMFSVKKIMEIHELVKLVNKWQNIAYKYDKDLLLMTHFIT | | |
| | RNITDNQGKNKTAIHTYFSSVFLGGVDSLVDLMNKSFPELGI | | |
| | KKTDCRQLSWIDTIIFYSGVVNYDTDNFNKEILLDRSAGQNG | | |
| | AFKIKLDYVKKPIPESVFVQILEKLYEEDIGAGMYALYPYGG | | |
| | IMDEISESAIPFPHRAGILYELWYICSWEKQEDNEKHLNWIR | | |
| | NIYNFMTPYVSKNPRLAYLNYRDLDIGINDPKNPNNYTQARI | | |
| | WGEKYFGKNFDRLVKVKTLVDPNNFFRNEQSIPPLPRHRH | | |
| CBDA synthase | NPRENFLKCFSQYIPNNATNLKLVYTQNNPLYMSVLNSTIHN | 13 | 14 |
| (d28_CBDAS) | LRFTSDTTPKPLVIVTPSHVSHIQGTILCSKKVGLQIRTRSG | | |
| [Cannabis | GHDSEGMSYISQVPFVIVDLRNMRSIKIDVHSQTAWVEAGAT | | |
| sativa] | LGEVYYWVNEKNENLSLAAGYCPTVCAGGHFGGGGYGPLMRN | | |
| (28 aa N-term | YGLAADNIIDAHLVNVHGKVLDRKSMGEDLFWALRGGGAESF | | |
| SP truncation) | GIIVAWKIRLVAVPKSTMFSVKKIMEIHELVKLVNKWQNIAY | | |
| | KYDKDLLLMTHFITRNITDNQGKNKTAIHTYFSSVFLGGVDS | | |
| | LVDLMNKSFPELGIKKTDCRQLSWIDTIIFYSGVVNYDTDNF | | |
| | NKEILLDRSAGQNGAFKIKLDYVKKPIPESVFVQILEKLYEE | | |
| | DIGAGMYALYPYGGIMDEISESAIPFPHRAGILYELWYICSW | | |
| | EKQEDNEKHLNWIRNIYNFMTPYVSKNPRLAYLNYRDLDIGI | | |
| | NDPKNPNNYTQARIWGEKYFGKNFDRLVKVKTLVDPNNFFRN | | |
| | EQSIPPLPRHRH | | |
| THCA synthase | MNCSAFSFWFVCKIIFFFLSFHIQISIANPRENFLKCFSKHI | 101 | 102 |
| (THCAS) | PNNVANPKLVYTQHDQLYMSILNSTIQNLRFISDTTPKPLVI | | |
| [Cannabis | VTPSNNSHIQATILCSKKVGLQIRTRSGGHDAEGMSYISQVP | | |
| sativa] | FVVVDLRNMHSIKIDVHSQTAWVEAGATLGEVYYWINEKNEN | | |
| BAC41356.1 | LSFPGGYCPTVGVGGHFSGGGYGALMRNYGLAADNIIDAHLV | | |
| | NVDGKVLDRKSMGEDLFWAIRGGGGENFGIIAAWKIKLVAVP | | |
| | SKSTIFSVKKNMEIHGLVKLFNKWQNIAYKYDKDLVLMTHFI | | |
| | TKNITDNHGKNKTTVHGYFSSIFHGGVDSLVDLMNKSFPELG | | |
| | IKKTDCKEFSWIDTTIFYSGVVNFNTANFKKEILLDRSAGKK | | |
| | TAFSIKLDYVKKPIPETAMVKILEKLYEEDVGAGMYVLYPYG | | |
| | GIMEEISESAIPFPHRAGIMYELWYTASWEKQEDNEKHINWV | | |
| | RSVYNFTTPYVSQNPRLAYLNYRDLDLGKTNHASPNNYTQAR | | |
| | IWGEKYFGKNFNRLVKVKTKVDPNNFFRNEQSIPPLPPHHH | | |
| THCA synthase | NPRENFLKCFSKHIPNNVANPKLVYTQHDQLYMSILNSTIQN | 103 | 104 |
| (d28_THCAS) | LRFISDTTPKPLVIVTPSNNSHIQATILCSKKVGLQIRTRSG | | |
| [Cannabis | GHDAEGMSYISQVPFVVVDLRNMHSIKIDVHSQTAWVEAGAT | | |
| sativa] | LGEVYYWINEKNENLSFPGGYCPTVGVGGHFSGGGYGALMRN | | |
| (28 aa N-term | YGLAADNIIDAHLVNVDGKVLDRKSMGEDLFWAIRGGGGENF | | |
| SP truncation) | GIIAAWKIKLVAVPSKSTIFSVKKNMEIHGLVKLFNKWQNIA | | |
| | YKYDKDLVLMTHFITKNITDNHGKNKTTVHGYFSSIFHGGVD | | |
| | SLVDLMNKSFPELGIKKTDCKEFSWIDTTIFYSGVVNFNTAN | | |
| | FKKEILLDRSAGKKTAFSIKLDYVKKPIPETAMVKILEKLYE | | |
| | EDVGAGMYVLYPYGGIMEEISESAIPFPHRAGIMYELWYTAS | | |
| | WEKQEDNEKHINWVRSVYNFTTPYVSQNPRLAYLNYRDLDLG | | |
| | KTNHASPNNYTQARIWGEKYFGKNFNRLVKVKTKVDPNNFFR | | |
| | NEQSIPPLPPHHH | | |

Although Table 4 lists AAE1 from C. sativa, as described elsewhere herein, the present disclosure provides advantages where the heterologous AAE incorporated in the pathway is from a plant source other than C. sativa. As is described elsewhere herein, the use of AAE enzymes other than AAE1 can result in an enhanced level of production of the cannabinoid precursor compounds, OA or DA relative to OA or DA production in recombinant cells comprising the corresponding pathway with the AAE1 enzyme from C. sativa of SEQ ID NO: 2. Moreover, this production of OA and/or DA can occur even when the host cells are cultured in the presence of an HA and/or BA feedstock. Thus, in at least one embodiment, the recombinant host cell of the present disclosure comprises a heterologous pathway of at least the enzymes AAE, OLS, and OAC, wherein AAE is not AAE1 from C. sativa.

In at least one embodiment, the heterologous pathway capable of producing a cannabinoid precursor comprises at least the enzymes AAE, OLS, and OAC, wherein the enzymes OLS and OAC have amino acid sequences of at least 90% identity to SEQ ID NO: 4 (OLS) and at least 90% identity to SEQ ID NO: 6 (OAC), respectively. The AAE enzyme from a plant source other than C. sativa used in the heterologous pathway of the host cell compositions and methods of the present disclosure have an amino acid sequence of at least 70%, at least 75%, at least 80%, at least 85%, at least 90%, at least 95%, at least 98%, or greater identity to a sequence selected from TM4 (SEQ ID NO: 16), CCL2 (SEQ ID NO: 18), CM1 (SEQ ID NO: 20), DA1 (SEQ ID NO: 22), CCL3 (SEQ ID NO: 24), AA1 (SEQ ID NO: 26), WC1 (SEQ ID NO: 28), CH3 (SEQ ID NO: 30), CH2 (SEQ ID NO: 32), PA1 (SEQ ID NO: 34), and TM5 (SEQ ID NO: 36).

In at least one embodiment, the heterologous pathway is capable of producing a cannabinoid precursor (e.g., OA and/or DA) and further comprises a prenyltransferase enzyme (e.g., PT4) having an amino acid sequence of at least 90% identity to SEQ ID NO: 8, that allows the pathway to further produce a cannabinoid (e.g., CBGA and/or CBGVA).

In at least one embodiment, wherein the heterologous pathway is capable of producing a cannabinoid and further comprises a prenyltransferase enzyme, the pathway further comprises a cannabinoid synthase enzyme of CBDAS, THCAS, and/or CBCAS, optionally, a CBDAS having an amino acid sequence of at least 90% identity to SEQ ID NO: 12. In such an embodiment, the heterologous pathway further comprising a cannabinoid synthase enzyme of CBDAS, THCAS, and/or CBCAS, is capable of further converting the cannabinoid compound, CBGA, to the cannabinoid compound, CBDA, THCA, and/or CBCA, and/or converting the rare cannabinoid compound, CBGVA, to the rare cannabinoid compound, CBDVA, THCVA, and/or CBCVA.

The sequences of the exemplary cannabinoid pathway enzymes AAE1, OLS, OAC, PT4, CBDAS, and THCAS listed in Table 4 are naturally occurring sequences derived from the plant source, Cannabis sativa. In the recombinant host cell embodiments of the present disclosure, it is contemplated that the polynucleotide encoding the AAE1 enzyme of SEQ ID NO: 2 is replaced in the host cell by an recombinant polynucleotide encoding a recombinant polypeptide having AAE activity from an organism other than C. sativa disclosed in Table 3, specifically an AAE enzyme having an amino acid sequence of at least 70%, at least 75%, at least 80%, at least 85%, at least 90%, at least 95%, at least 98%, or greater identity to a sequence selected from SEQ ID NO: 16, 18, 20, 22, 24, 26, 28, 30, 32, 34, and 36.

It is contemplated that the other heterologous cannabinoid pathway enzymes used in the recombinant host cell can include enzymes derived from naturally occurring sequence homologs of the Cannabis sativa enzymes, OLS, OAC, PT4, CBDAS, THCAS, and/or CBCAS. For example, based on the sequence, accession, and enzyme classification information provided herein, one of ordinary skill can identify known naturally occurring homologs to OLS, OAC, PT4, CBDAS, THCAS, CBCAS having activity in the desired biocatalytic reaction.

Additionally, it is contemplated that the pathway enzymes OLS, OAC, PT4, CBDAS, THCAS, and/or CBCAS, as used in a recombinant host cell including an engineered gene of the present disclosure can include enzymes having non-naturally occurring sequences. For example, enzymes with amino acid sequences engineered to function optimally in a particular enzyme pathway, and/or optimally for production of particular cannabinoid, and/or optimally in a particular host. Methods for preparing such non-naturally occurring enzyme sequences are known in the art and include methods for enzyme engineering such as directed evolution (see, e.g., Stemmer, 1994, Proc Natl Acad Sci USA 91:10747-10751; PCT Publ. Nos. WO 95/22625, WO 97/0078, WO 97/35966, WO 98/27230, WO 00/42651, and WO 01/75767; U.S. Pat. Nos. 6,537,746; 6,117,679; 6,376,246; and 6,586,182; and U.S. Pat. Publ. Nos. 20080220990A1 and 20090312196A1; each of which is hereby incorporated by reference herein). Other modifications of cannabinoid pathway enzymes contemplated by the present disclosure include modification of the enzyme's amino acid sequence at either its N- or C-terminus by truncation or fusion. For example, in at least one embodiment of the pathway of producing a cannabinoid, versions of the OLS, OAC PT4, CBDAS, THCAS, and/or CBCAS enzymes that are engineered with amino acid substitutions and/or truncated at the N- or C-terminus can be prepared using methods known in the art, and used in the compositions and methods of the present disclosure. For example, in one embodiment, a CBDAS enzyme of SEQ ID NO: 12 that is truncated at the N-terminus by 28 amino acids to delete the native signal peptide can be used. The amino acid sequence of such a truncated CBDAS is provided herein as the d28_CBDAS enzyme of SEQ ID NO: 14. Accordingly, in at least one embodiment of the recombinant host cell, the pathway capable of producing a cannabinoid precursor or cannabinoid comprises at least enzymes having an amino acid sequence at least 90% identity to SEQ ID NO: 4 (OLS), SEQ ID NO: 6 (OAC), SEQ ID NO: 8 (d82_PT4), and an amino acid sequence of at least 90% identity to a recombinant polypeptide having AAE activity of the present disclosure as provided in Tables 3, and the accompanying Sequence Listing. Additionally, in at least one embodiment of the recombinant host cell, the pathway capable of producing a cannabinoid can further comprise a cannabinoid synthase of SEQ ID NO: 14 (d28_CBDAS) and/or SEQ ID NO: 104 (d28_THCAS).

The recombinant polypeptides having AAE activity encoded by the genes of the present disclosure when integrated into recombinant host cells with a pathway capable of converting hexanoic acid (HA) to the C-12 tetraketide-CoA precursor, 3,5,7-trioxododecanoyl-CoA, can provide enhanced yields of the cannabinoid precursor, OA, which can be further converted to the cannabinoids, CBGA, CBDA, THCA, etc. It is contemplated that any of the genes encoding AAE enzymes of the present disclosure (e.g., AAE enzymes of Table 3) that encode recombinant polypeptides having AAE activity can be incorporated into a four or five enzyme cannabinoid pathway as depicted in FIG. 1 and FIG. 2 to express the AAE activity needed for the biosynthesis of cannabinoid precursor, OA, and its downstream cannabinoid products, CBGA, CBDA, THCA, and/or CBCA. Accordingly, in at least one embodiment, the present disclosure provides a recombinant host cell comprising recombinant polynucleotides encoding a pathway capable of producing a cannabinoid, wherein the pathway comprises enzymes capable of catalyzing reactions (i)-(iv):

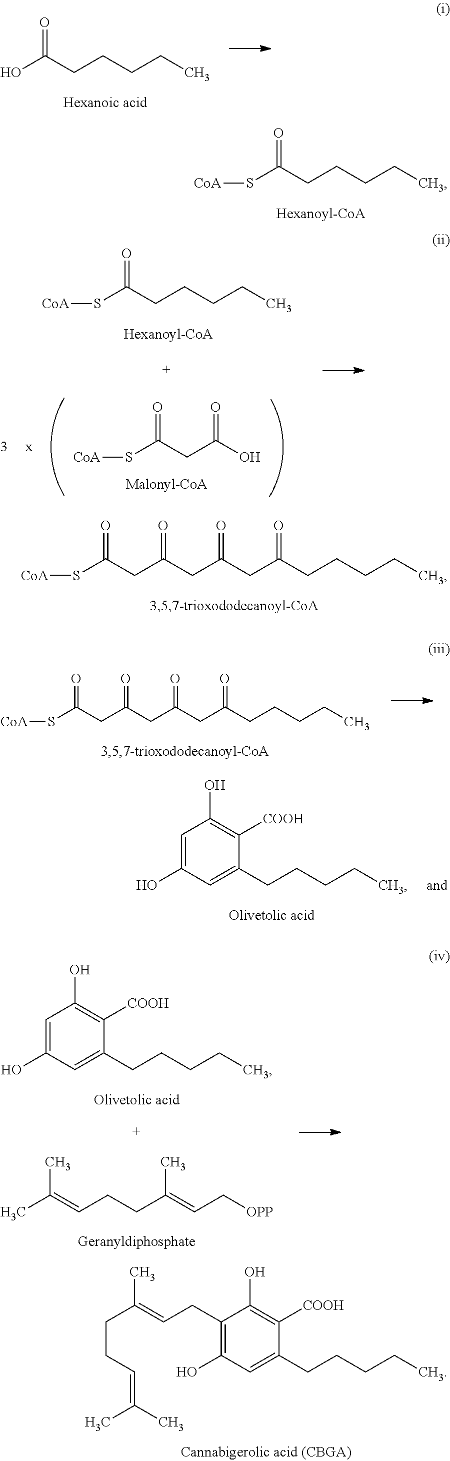

As shown in FIG. 1, exemplary enzymes capable of catalyzing reactions (i)-(iv) are: (i) acyl activating enzyme (AAE); (ii) olivetol synthase (OLS); (iii) olivetolic acid cyclase (OAC); and (iv) prenyltransferase (PT). In at least one embodiment, the AAE of the pathway of the recombinant host cell is a recombinant polypeptide having AAE activity of the present disclosure, such as an exemplary recombinant polypeptides disclosed in Table 3.

In at least one embodiment, it is contemplated that a recombinant host cell comprising a pathway comprising the two enzymes, OAC, and OLS, could be modified by integrating a recombinant polynucleotide encoding an AAE enzyme of the present disclosure to provide expression of a three enzyme pathway to the cannabinoid precursor, OA, as illustrated by the first three steps depicted FIG. 1 corresponding to the reactions (i)-(iii) above.

As shown in FIG. 2, the cannabinoid compound, CBGA, that is produced by the pathway of FIG. 1, can be further converted by a cannabinoid synthase to at least three other different cannabinoid compounds, Δ⁹-tetrahydrocannabinolic acid (THCA), cannabidiolic acid (CBDA), and/or cannabichromenic acid (CBCA). Accordingly, in at least one embodiment, the present disclosure provides a recombinant host cell comprising a pathway capable of converting hexanoic acid to CBGA and further comprising an enzyme capable of catalyzing the conversion of (v) CBGA to Δ⁹-THCA; (vi) CBGA to CBDA; and/or (vii) CBGA to CBCA. Thus, in at least one embodiment, the recombinant host cell comprises pathway capable of converting hexanoic acid to CBGA further comprises further comprises enzymes capable of catalyzing a reaction (v), (vi), and/or (vii):

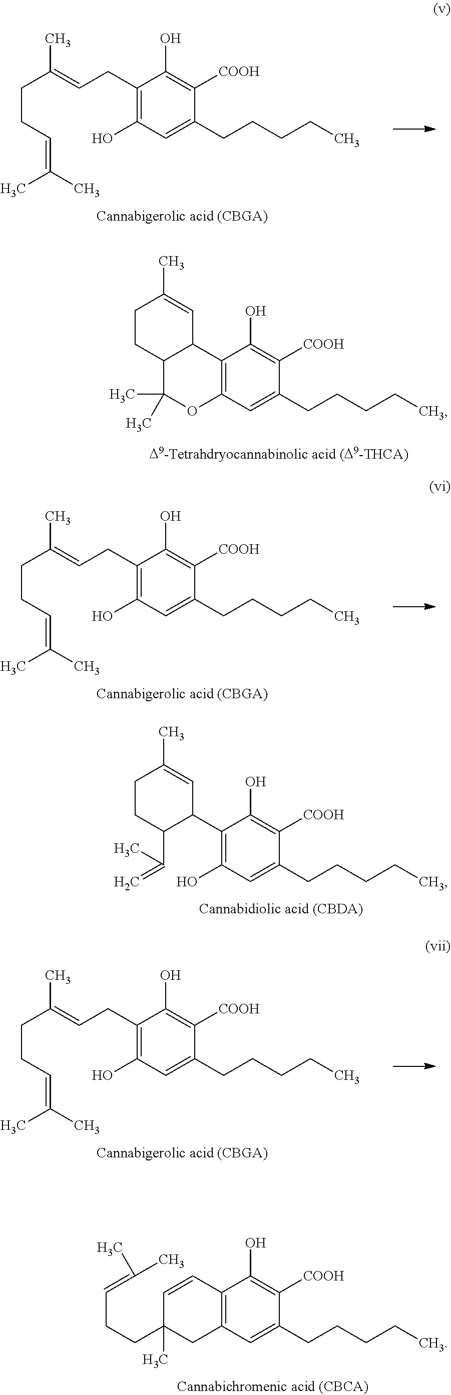

As shown in FIG. 2, exemplary enzymes capable of catalyzing reaction (v)-(vii) are: (v) THCA synthase (THCAS); (vi) CBDA synthase (CBDAS); and (vii) CBCA synthase (CBCAS). The extension of the four enzyme exemplary pathway of FIG. 1 with polynucleotide sequence capable of expressing such a cannabinoid synthase (e.g., CBDAS, THCAS, and/or CBCAS) allows for the biosynthetic production of one or more of the cannabinoids, Δ⁹-THCA, CBDA, and/or CBCA. These cannabinoids can then be decarboxylated to provide the cannabinoids, Δ⁹-THC, CBD, and/or CBC. Accordingly, it is contemplated, that in some embodiments this further decarboxylation reaction can be carried out under in vitro reaction conditions using the cannabinoid acids separated and/or isolated from the recombinant host cells.

Other cannabinoid pathway enzymes useful in the recombinant host cells and associated methods of the present disclosure are known in the art, and can include naturally occurring enzymes obtained or derived from cannabis plants, or non-naturally occurring enzymes that have been engineered based on the naturally occurring cannabis plant sequences. It is also contemplated that enzymes obtained or derived from other organisms (e.g., microorganisms) having a catalytic activity related to a desired conversion activity useful in a cannabinoid pathway can be engineered for use in a recombinant host cell of the present disclosure.

A wide range of cannabinoid compounds can be produced biosynthetically by a recombinant host cell integrated with such a cannabinoid pathway. The cannabinoid pathways of FIGS. 1-2 depict the production of the more common naturally occurring cannabinoids, CBGA, Δ⁹-THCA, CBDA, and CBCA. It is also contemplated, however, that the engineered genes, recombinant polypeptides, cannabinoid pathways, recombinant host cells, and associated methods of the present disclosure can also be used to biosynthesize a range of additional rarely occurring, and/or synthetic cannabinoid compounds. Table 1 (above) lists the names and depicts the chemical structures of a wide range of exemplary rarely occurring, and/or synthetic cannabinoid compounds (e.g., CBGVA, CBDVA, THCVA) that are contemplated for production using the recombinant polypeptides, host cells, compositions, and methods of the present disclosure.

Similarly, Table 2 (above) depicts additional rarely occurring, and/or synthetic cannabinoid precursor compounds (e.g., DA) that could be produced by such recombinant host cells in the pathway for production of certain rarely occurring, and/or synthetic cannabinoid compounds of Table 1. Accordingly, in at least one embodiment, a recombinant host cell that includes a pathway to a cannabinoid precursor and that expresses a recombinant polypeptide having OAC activity of the present disclosure (e.g., as in Tables 3, 5, or 6) can be used for the biosynthetic production of a rarely occurring, and/or synthetic cannabinoid compound, or a composition comprising such a cannabinoid compound. It is contemplated that the produced rarely occurring, and/or synthetic cannabinoid precursors and cannabinoids can include, but is not limited to, the compounds listed in Tables 1 and 2. Accordingly, in at least embodiment, a recombinant host cell of the present disclosure can be used for production of a cannabinoid compound selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

In at least one embodiment, the compositions and methods of the present disclosure can be used for the production of the rare varin series of cannabinoids, CBGVA, Δ⁹-THCVA, CBDVA, and CBCVA, and cannabinoid precursor, DA. As shown in Table 1, the varin cannabinoids feature a 3 carbon propyl side-chain rather than the 5 carbon pentyl side chain found in the common cannabinoids, CBGA, Δ⁹-THCA, CBDA, and CBCA. An exemplary cannabinoid pathway capable of producing the rare naturally occurring cannabinoid, cannabigerovarinic acid (CBGVA), is depicted in FIG. 3. Instead of starting with hexanoic acid, the pathway of FIG. 3 is fed butyric acid (BA) which is converted to cannabinoid precursor, divarinic acid (DA) via the same three enzyme pathway of AAE, OLS, and OAC. The cannabinoid precursor DA is then converted by an prenyltransferase to the rare cannabinoid, CBGVA.

As described elsewhere herein, it is an unexpected and surprising advantage of the heterologous cannabinoid pathway comprising an AAE enzyme derived from a plant source other than C. sativa as disclosed herein, that it can produce a rare cannabinoid precursor or cannabinoid in greater amounts than the same heterologous pathway with the AAE1 enzyme from C. sativa. In at least one embodiment, the recombinant host cell comprising a recombinant AAE enzyme derived from a plant source organism other than C. sativa is capable of producing the cannabinoid with a titer that is increased relative to a control recombinant host cell comprising the same cannabinoid biosynthesis pathway but with the AAE enzyme of AAE1 from C. sativa. In at least one embodiment, the titer of cannabinoid produced is increased by at least 1.1-fold. 1.2-fold, 1.3-fold, 1.4-fold, 1.5-fold, 2-fold, 3-fold, 4-fold, 5-fold, 6-fold, 10-fold, or more relative to a control recombinant host cell that comprises the pathway with AAE1 from C. sativa.

In at least one embodiment, the recombinant host cell of the present disclosure comprises a pathway capable of producing a cannabinoid precursor DA from BA substrate feedstock, wherein the pathway comprises enzymes capable of catalyzing reactions (i)-(iii):

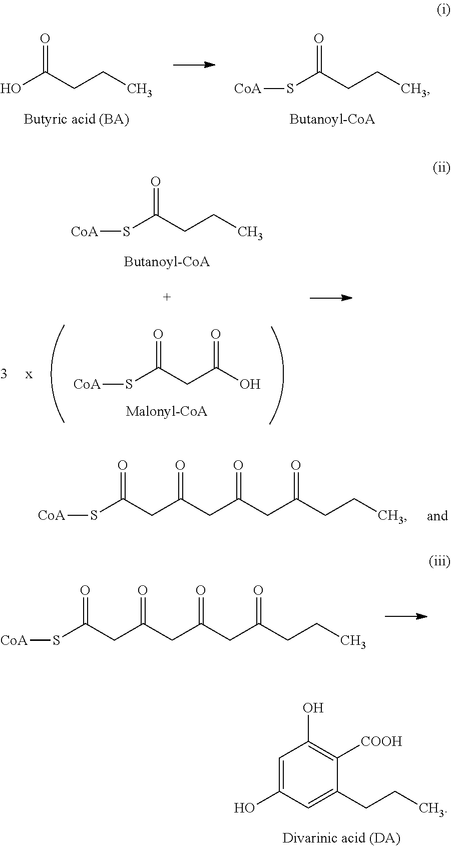

In at least one embodiment, the recombinant pathway comprises at least enzymes capable of producing DA from BA, and then converting DA to the rare varin cannabinoid, CBGVA. One such a pathway capable of converting BA to CBGVA is illustrated in FIG. 3. Accordingly, in at least one embodiment of the recombinant host cell, the pathway capable of producing a cannabinoid comprises enzymes capable of catalyzing reactions (i)-(iv):

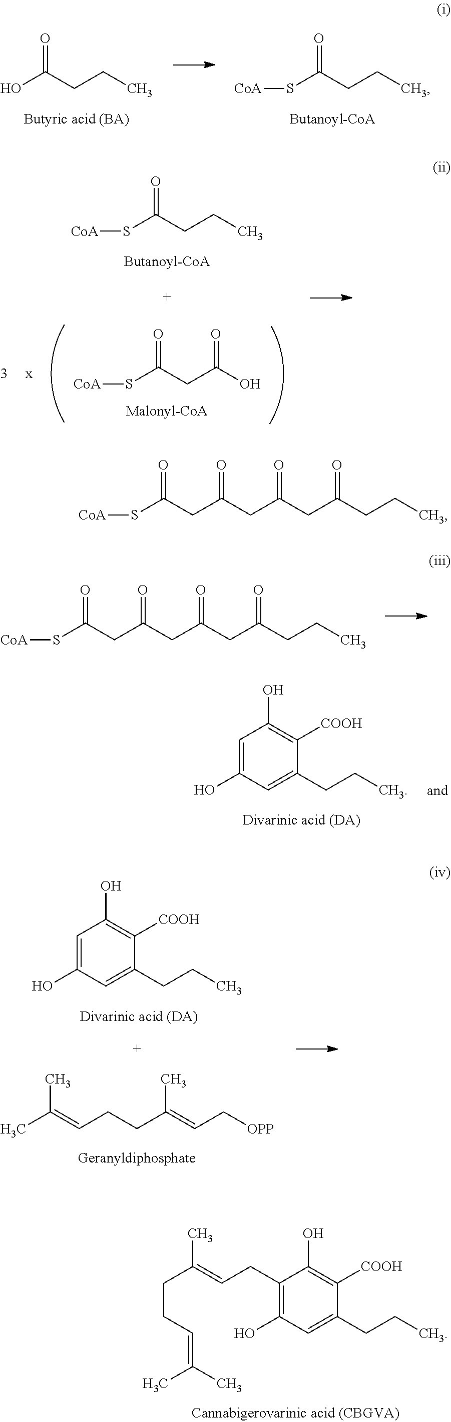

As shown in FIG. 3, exemplary enzymes capable of catalyzing reactions (i)-(iv) are: (i) acyl activating enzyme (AAE); (ii) olivetol synthase (OLS); (iii) olivetolic acid cyclase (OAC); and (iv) aromatic prenyltransferase (PT4). Exemplary AAE enzymes derived from plant sources other than C. sativa are provided in Table 3. Exemplary enzymes, OLS, OAC, and PT4 derived from C. sativa are known in the art and also provided in Table 4 and the accompanying Sequence Listing.

As shown in FIG. 4, the rare varin cannabinoid compound, CBGVA, that is produced by the pathway of FIG. 3, can be further converted to at least three other rare cannabinoid compounds, cannabidivarinic acid (CBDVA), Δ⁹-tetrahydrocannabivarinic acid (Δ⁹-THCVA), and cannabichromevarinic acid (CBCVA). Accordingly, in at least one embodiment, the present disclosure provides a recombinant host cell comprising a pathway capable of converting BA to CBGVA and further comprising an enzyme capable of catalyzing the conversion of (v) CBGVA to Δ⁹-THCVA; (vi) CBGVA to CBDVA; and/or (vii) CBGVA to CBCVA. Thus, in at least one embodiment, the recombinant host cell comprises pathway capable of converting BA to CBGVA further comprises further comprises enzymes capable of catalyzing a reaction (v), (vi), and/or (vii):

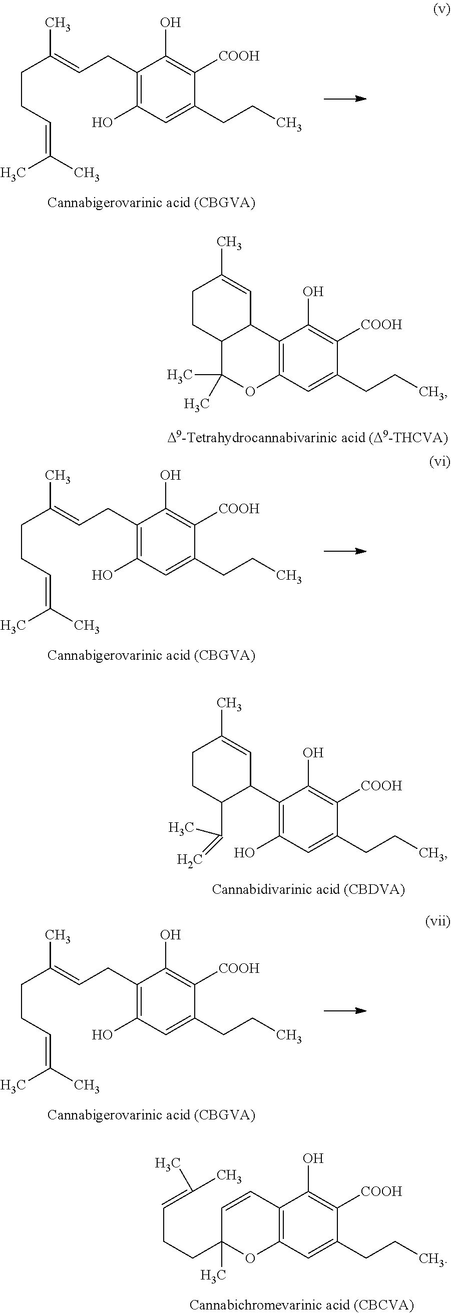

As shown in FIG. 4, exemplary enzymes capable of catalyzing reaction (v)-(vii) are: (v) THCA synthase (THCAS); (vi) CBDA synthase (CBDAS); and (vii) CBCA synthase (CBCAS). Exemplary THCAS, CBDAS, and CBCAS enzymes are provided in Table 4.

As described elsewhere herein, the following AAE enzymes from a plant source other than C. sativa were screened and found to be capable of producing the rare cannabinoid precursor, DA, and/or the rare cannabinoid, CBGVA when incorporated in the heterologous pathway of a host cell: TM4 (SEQ ID NO: 16), CCL2 (SEQ ID NO: 18), CM1 (SEQ ID NO: 20), DA1 (SEQ ID NO: 22), CCL3 (SEQ ID NO: 24), AA1 (SEQ ID NO: 26), WC1 (SEQ ID NO: 28), CH3 (SEQ ID NO: 30), CH2 (SEQ ID NO: 32), PA1 (SEQ ID NO: 34), and TM5 (SEQ ID NO: 36). As described in the Examples, in some embodiments, these AAE enzymes were observed to provide at least 1.5-fold improvement of CBGVA production in a recombinant host cell system that converts BA to DA and then to CBGVA via a pathway comprising the enzymes AAE, OLS, OAC and PT4 (see e.g., FIG. 3).

In at least one embodiment, the present disclosure also provides a recombinant host cell comprising a pathway capable of producing a rare cannabinoid, wherein the pathway comprises the enzymes AAE, OLS, OAC, and optionally PT4, and the AAE enzyme has an amino acid sequence having at least 70%, at least 75%, at least 80%, at least 85%, at least 90%, at least 95%, at least 98%, or greater identity to a sequence selected from SEQ ID NO: 16, 18, 20, 22, 24, 26, 28, 30, 32, 34, and 36. In at least one embodiment, the AAE enzyme comprises the amino acid sequence of any one of SEQ ID NO: 16, 18, 20, 22, 24, 26, 28, 30, 32, 34, and 36. In at least one embodiment, the AAE enzyme is encoded by a nucleic acid sequence having at least 70%, at least 75%, at least 80%, at least 85%, at least 90%, at least 95%, at least 98%, or greater identity to a sequence selected from SEQ ID NO: 15, 17, 19, 21, 23, 25, 27, 29, 31, 33, and 35. In at least one embodiment, the nucleic acid encoding the AAE enzyme comprises a nucleotide sequence of any one of SEQ ID NO: 15, 17, 19, 21, 23, 25, 27, 29, 31, 33, and 35.

In at least one embodiment, the present disclosure provides an isolated nucleic acid, wherein the nucleic acid encodes a pathway comprising the enzymes AAE, OLS, OAC, and optionally PT4, wherein the AAE enzyme comprises an amino acid sequence having at least 70%, at least 75%, at least 80%, at least 85%, at least 90%, at least 95%, at least 98%, or greater identity to a sequence selected from SEQ ID NO: 16, 18, 20, 22, 24, 26, 28, 30, 32, 34, and 36. In at least one embodiment, the nucleic acid encoding the pathway comprising the enzymes AAE, OLS, OAC, and optionally PT4, the portion of the nucleic acid encoding the AAE enzyme encodes an amino acid sequence of any one of SEQ ID NO: 16, 18, 20, 22, 24, 26, 28, 30, 32, 34, and 36. In at least one embodiment, the nucleotide sequence of the nucleic acid encoding the pathway is codon-optimized for expression in a recombinant host cell, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli.

In at least one embodiment, the present disclosure provides an isolated nucleic acid, wherein the nucleic acid encodes a pathway comprising the enzymes AAE, OLS, OAC, and optionally PT4, wherein the portion of the nucleic acid encoding the AAE enzyme comprises a nucleotide sequence having at least 70%, at least 75%, at least 80%, at least 85%, at least 90%, at least 95%, at least 98%, or greater identity to a sequence selected from SEQ ID NO: 15, 17, 19, 21, 23, 25, 27, 29, 31, 33, and 35. In at least one embodiment, the nucleic acid encoding the pathway comprising the enzymes AAE, OLS, OAC, and optionally PT4, the portion of the nucleic acid encoding the AAE enzyme comprises a nucleotide sequence of any one of SEQ ID NO: 15, 17, 19, 21, 23, 25, 27, 29, 31, 33, and 35.

In at least one embodiment, the present disclosure provides a vector comprising a heterologous nucleic acid encoding a pathway comprising the enzymes AAE, OLS, OAC, and optionally PT4, wherein the portion of the nucleic acid encoding the AAE enzyme encodes an amino acid sequence having at least 70%, at least 75%, at least 80%, at least 85%, at least 90%, at least 95%, at least 98%, or greater identity to a sequence selected from SEQ ID NO: 16, 18, 20, 22, 24, 26, 28, 30, 32, 34, and 36. In at least one embodiment, the vector comprises a nucleic acid that is codon-optimized for expression in a recombinant host cell, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli.

In at least one embodiment, the present disclosure provides a vector comprising a heterologous nucleic acid encoding a pathway comprising the enzymes AAE, OLS, OAC, and optionally PT4, wherein the portion of the nucleic acid encoding the AAE enzyme comprises a nucleotide sequence having at least 70%, at least 75%, at least 80%, at least 85%, at least 90%, at least 95%, at least 98%, or greater identity to any one of SEQ ID NO: 15, 17, 19, 21, 23, 25, 27, 29, 31, 33, and 35.

In at least one embodiment, the nucleic acids and vectors encoding pathway capable of producing a rare cannabinoid of the present disclosure comprise the enzymes AAE, OLS, OAC, and optionally PT4, wherein the enzymes OLS, OAC, and PT4, have amino acid sequences of at least 90% sequence identity to SEQ ID NO: 4 (OLS), SEQ ID NO: 6 (OAC), and SEQ ID NO: 8 (PT4) or 10 (d82_PT4), respectively, and the enzyme AAE has an amino acid sequence of at least 70%, at least 75%, at least 80%, at least 85%, at least 90%, at least 95%, at least 98% identity to a sequence selected from SEQ ID NO: 16, 18, 20, 22, 24, 26, 28, 30, 32, 34, and 36 In at least one embodiment, the nucleotide sequences encoding the pathway of enzymes are codon-optimized for expression in a recombinant host cell, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli.

Some of the amino acid sequences of the AAE, OLS, OAC, PT4, CBDAS, and/or THCAS enzymes are selected from SEQ ID NO: 2, 4, 6, 8, 10, 12, 14, 16, 18, 20, 22, 24, 26, 28, 30, 32, 34, and 36, provided in the present disclosure begin with an initiating methionine (M) residue at position 1, although it will be understood by the skilled artisan that this initiating methionine residue may be removed by biological processing machinery, such as in a host cell or in vitro translation system, to generate a mature protein lacking the initiating methionine residue. Accordingly, it is contemplated that in any embodiment of the present disclosure comprising an amino acid sequence of an AAE, OLS, OAC, PT4, CBDAS, and/or THCAS enzyme can comprise an amino acid sequence selected from SEQ ID NO: 2, 4, 6, 8, 10, 12, 14, 16, 18, 20, 22, 24, 26, 28, 30, 32, 34, and 36, wherein the methionine residue at position 1 is deleted.

As described herein, the heterologous cannabinoid pathways of the present disclosure can be incorporated into a range of host cells to provide a system for biosynthetic production of cannabinoids (e.g., CBGA, CBGVA, CBDA, CBDVA, THCA, THCVA). Methods and techniques for integrating polynucleotides into recombinant host cells, such as yeast, so that they express functional pathways of enzymes are well known in the art and described elsewhere herein including the Examples. Generally, the host cell source used in the recombinant host cell of the present disclosure can be any cell that can be recombinantly modified with nucleic acids and express the recombinant products of those nucleic acids, including polypeptides and metabolites produced by the activity of the recombinant polypeptides. A wide range of suitable sources of host cells are known in the art, and exemplary host cell sources useful as recombinant host cells of the present disclosure include, but are not limited to, Saccharomyces cerevisiae, Yarrowia lipolytica, Pichia pastoris, and Escherichia coli. It is also contemplated that the host cell source for a recombinant host cell of the present disclosure can include a non-naturally occurring cell source, e.g., an engineered host cell. For example, a non-naturally occurring source host cell, such as a yeast cell previously engineered for improved production of recombinant genes, may be used to prepare the recombinant host cell of the present disclosure. Accordingly, in at least one embodiment, the present disclosure provides a recombinant host cell transformed with a cannabinoid biosynthesis pathway and a heterologous nucleic acid encoding a protein that is not part of the pathway, wherein the host cell source is selected from Saccharomyces cerevisiae, Yarrowia lipolytica, Pichia pastoris, Escherichia coli, or an engineered cell derived from Saccharomyces cerevisiae, Yarrowia lipolytica, Pichia pastoris, Escherichia coli.

The recombinant hosts of the present disclosure comprise heterologous nucleic acids encoding a pathway of enzymes capable of producing a cannabinoid, wherein the heterologous nucleic acids comprise sequence encoding an AAE enzyme from a plant source other than C. sativa. As described elsewhere herein, nucleic acid sequences encoding AAE enzymes, and the other cannabinoid pathway enzymes, are known in the art and provided herein and can readily be used in accordance with the present disclosure. Typically, the nucleic acid sequence encoding enzymes which form a part of a cannabinoid pathway, further include one or more additional nucleic acid sequences, for example, a nucleic acid sequence controlling expression of the proteins which form a part of a cannabinoid biosynthetic enzyme pathway, and these one or more additional nucleic acid sequences together with the nucleic acid sequence encoding a protein which form a part of an cannabinoid biosynthetic enzyme pathway can be considered a heterologous nucleic acid sequence. A variety of techniques and methodologies are available and well known in the art for introducing heterologous nucleic acid sequences, such as nucleic acid sequences encoding the AAE enzymes, into a host cell so as to attain expression of a AAE in a cannabinoid pathway. Such techniques are well known to the skilled artisan and can, for example, be found in Sambrook et al., Molecular Cloning, a Laboratory Manual, Cold Spring Harbor Laboratory Press, 2012, Fourth Ed.

One of ordinary skill will recognize that the heterologous nucleic acids encoding the AAE enzyme (and other pathway enzymes) will further comprise transcriptional promoters capable of controlling expression of the enzymes in the recombinant host cell. Generally, the transcriptional promoters are selected to be compatible with the host cell, so that promoters obtained from bacterial cells are used when a bacterial host cell is selected in accordance herewith, while a fungal promoter is used when a fungal host cell is selected, a plant promoter is used when a plant cell is selected, and so on. Promoters useful in the recombinant host cells of the present disclosure may be constitutive or inducible, provided such promoters are operable in the host cells.

Promoters that may be used to control expression in fungal host cells, such as Saccharomyces cerevisiae, Yarrowia lipolytica, Pichia pastoris, and Komagataella phaffii, are well known in the art and include, but are not limited to: inducible promoters, such as a GAL1 promoter or GAL10 promoter, a constitutive promoter, such as an alcohol dehydrogenase (ADH) promoter, a glyceraldehyde-3-phosphate dehydrogenase (GPD) promoter, an S. pombe Nmt, or ADH promoter, or any of the known Saccharomyces cerevisiae promoters that are commonly used to control expression of recombinant genes, including but not limited to, ALD6, HHF1, HTB2, PAB1, POP6, PSP2, RAD27, RET2, REV1, RNR1, RNR2, RPL18B, SAC6, STE5, TDH3, CCW12, HHF2, PGK1, TEF1, and TEF2. In at least one embodiment of the present disclosure, wherein recombinant host cell is yeast, the gene encoding the AAE enzyme (not from C. sativa) in the cannabinoid pathway is under control of the promoter ALD6. It is contemplated that the fungal host cell can comprise multiple copies of a cannabinoid pathway comprising AAE, OLS, OAC, and optionally, PT4, THCAS, CBDAS, or CBCAS enzymes, integrated in the hosts genome. In some embodiments, each of the multiple copies would be integrated at a different genomic loci. In at least one embodiment of the recombinant host cells of the present disclosure, the fungal host cell is Saccharomyces cerevisiae and the cell comprises at least three copies of a cannabinoid pathway comprising at least the AAE, OLS, and OAC enzymes. In at least one embodiment, the gene encoding the AAE enzyme in each copy of the pathway is under the control of an ALD6 promoter.

Exemplary promoters that may be used to control expression in bacterial cells can include the Escherichia coli promoters lac, tac, trc, trp or the T7 promoter. Exemplary promoters that may be used to control expression in plant cells include, for example, a Cauliflower Mosaic Virus 35S promoter (Odell et al. (1985) Nature 313:810-812), a ubiquitin promoter (U.S. Pat. No. 5,510,474; Christensen et al. (1989)), or a rice actin promoter (McElroy et al. (1990) Plant Cell 2:163-171). Exemplary promoters that can be used in mammalian cells include, a viral promoter such as an SV40 promoter or a metallothionine promoter. All of these host cell promoters are well known by and readily available to one of ordinary skill in the art. Further nucleic acid control elements useful for controlling expression in a recombinant host cell can include transcriptional terminators, enhancers and the like, all of which may be used with the heterologous nucleic acids incorporate in the recombinant host cells of the present disclosure.

A wide variety of techniques are well known in the art for linking transcriptional promoters and other control elements to heterologous nucleic acid sequences encoding pathway genes. Such techniques are described in e.g., Sambrook et al., Molecular Cloning, a Laboratory Manual, Cold Spring Harbor Laboratory Press, 2012, Fourth Ed. Accordingly, in at least one embodiment, the heterologous nucleic acid sequences of the present disclosure comprise a promoter capable of controlling expression in a host cell, wherein the promoter is linked to a nucleic acid sequence encoding an AAE enzyme, and, as necessary, other enzymes constituting a cannabinoid pathway (e.g., OLS, OAC, PT4). This heterologous nucleic acid sequence can be integrated into a recombinant expression vector which ensures good expression in the desired host cell, wherein the expression vector is suitable for expression in a host cell, meaning that the recombinant expression vector comprises the heterologous nucleic acid sequence linked to any genetic elements required to achieve expression in the host cell. Genetic elements that may be included in the expression vector in this regard include a transcriptional termination region, one or more nucleic acid sequences encoding marker genes, one or more origins of replication, and the like. In some embodiments, the expression vector further comprises genetic elements required for the integration of the vector or a portion thereof in the host cell's genome.

It is also contemplated that in some embodiments an expression vector comprising a heterologous nucleic acid of the present disclosure may further contain a marker gene. Marker genes useful in accordance with the present disclosure include any genes that allow the distinction of transformed cells from non-transformed cells, including all selectable and screenable marker genes. A marker gene may be a resistance marker such as an antibiotic resistance marker against, for example, kanamycin or ampicillin. Screenable markers that may be employed to identify transformants through visual inspection include β-glucuronidase (GUS) (U.S. Pat. Nos. 5,268,463 and 5,599,670) and green fluorescent protein (GFP) (Niedz et al., 1995, Plant Cell Rep., 14: 403).

As described elsewhere herein, the present disclosure provides recombinant host cells capable of producing a rare cannabinoid precursor, such as DA, or a rare cannabinoid, such as CBGVA, or CBDVA, wherein the host cell comprises a pathway of at least the enzymes AAE, OLS, OAC, and optionally, PT4, wherein the AAE enzyme is derived from a plant source other than C. sativa. In at least one embodiment, the AAE enzyme comprises an amino acid sequence having at least 70%, at least 75%, at least 80%, at least 85%, at least 90%, at least 95%, at least 98%, or greater identity to a sequence selected from SEQ ID NO: 16, 18, 20, 22, 24, 26, 28, 30, 32, 34, and 36. Such recombinant host cells are capable of producing the rare cannabinoid precursor or rare cannabinoid with a titer that is increased (e.g., 1.5-fold or more) relative to a control recombinant host cell comprising the same pathway but with the AAE enzyme, AAE1 from C. sativa comprising SEQ ID NO: 2. Accordingly, the recombinant host cell of the present disclosure can be used for improved biosynthetic production of rare cannabinoid precursors and rare cannabinoid compounds, as well as other cannabinoid compounds, including, but not limited to, the exemplary cannabinoid compounds provided in Table 1.

In at least one embodiment, the present disclosure provides a method for producing a cannabinoid precursor or cannabinoid comprising: (a) culturing in a suitable medium a recombinant host cell of the present disclosure; and (b) recovering the produced cannabinoid precursor or cannabinoid. In at least one embodiment of the method for producing a cannabinoid precursor or cannabinoid, a heterologous nucleic acid encoding an AAE enzyme derived from a plant source other than C. sativa, such as an AAE enzyme of Table 3, can be introduced into a recombinant host cell comprising a pathway capable of producing a cannabinoid precursor or cannabinoid to provide an recombinant host cell that has improved biosynthesis of the cannabinoid precursor or cannabinoid in terms of titer, yield, and production rate. Further description of preparation recombinant host cells with an integrated nucleic acid encoding an AAE enzyme capable of producing a cannabinoid or cannabinoid precursor are provided elsewhere herein including the Examples.

In at least one embodiment, a recombinant host cell of the present disclosure can be used to produce a rare cannabinoid selected from cannabidivarinic acid (CBDVA), cannabidivarin (CBDV), Δ⁹-tetrahydrocannabivarinic acid (Δ⁹-THCVA), Δ⁹-tetrahydrocannabivarin (Δ⁹-THCV), cannabichromevarinic acid (CBCVA), cannabichromevarin (CBCV), cannabigerovarinic acid (CBGVA), cannabigerovarin (CBGV), and any combination thereof. However, it is also contemplated that the recombinant host cells of the present disclosure can be used to produce other cannabinoids of Table 1 that do not include a varin group, including any of the cannabinoids selected from cannabigerolic acid (CBGA), cannabigerol (CBG), cannabidiolic acid (CBDA), cannabidiol (CBD), Δ⁹-tetrahydrocannabinolic acid (Δ⁹-THCA), Δ⁹-tetrahydrocannabinol (Δ⁹-THC), Δ⁸-tetrahydrocannabinolic acid (Δ⁸-THCA), Δ⁸-tetrahydrocannabinol (Δ⁸-THC), cannabichromenic acid (CBCA), cannabichromene (CBC), cannabinolic acid (CBNA), cannabinol (CBN), cannabidibutolic acid (CBDBA), cannabidibutol (CBDB), Δ⁹-tetrahydrocannabutolic acid (Δ⁹-THCBA), Δ⁹-tetrahydrocannabutol (Δ⁹-THCB), cannabidiphorolic acid (CBDPA), cannabidiphorol (CBDP), Δ⁹-tetrahydrocannabiphorolic acid (Δ⁹-THCPA), Δ⁹-tetrahydrocannabiphorol (Δ⁹-THCP), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

It is also contemplated that the method for producing a cannabinoid precursor or cannabinoid of the present disclosure can further comprise contacting a cell-free extract of the culture containing the produced cannabinoid precursor or cannabinoid with a biocatalytic reagent or chemical reagent. In such an embodiment of the method, the biocatalytic reagent used can be an enzyme capable of converting the produced cannabinoid precursor or cannabinoid to a different cannabinoid or a cannabinoid derivative compound. In another such embodiment of the method, the chemical reagent is capable of chemically modifying the produced cannabinoid precursor or cannabinoid can be used to produce a derivative compound of the cannabinoid precursor or cannabinoid. Accordingly, in at least one embodiment of the method, the recombinant host cell with improved cannabinoid precursor or cannabinoid production in terms of titer, yield, and production rate can be used in the production of a cannabinoid precursor or cannabinoid (e.g., compounds of Tables 2 and 1), or a derivative compound of a cannabinoid precursor or cannabinoid. Such derivative compounds of cannabinoid precursor compounds or cannabinoid compounds can include a wide range of naturally-occurring and non-naturally occurring compounds.

For example, cannabinoid derivative compounds produced using the recombinant host cells and methods of the present disclosure can include any compound structurally related to a cannabinoid compound (e.g., compounds of Table 1) but which lacks one or more of the chemical moieties present in the cannabinoid compound from which it derives. Exemplary chemical moieties that may be lacking in a cannabinoid derivative include, but are not limited to, methyl, alkyl, alkenyl, methoxy, alkoxy, acetyl, carboxyl, carbonyl, oxo, ester, hydroxyl, aryl, heteroaryl, cycloalkyl, cycloalkenyl, cycloalkylalkenyl, cycloalkenylalkyl, cycloalkenylalkenyl, heterocyclylalkenyl, heteroarylalkenyl, arylalkenyl, heterocyclyl, aralkyl, cycloalkylalkyl, heterocyclylalkyl, heteroarylalkyl, and the like.

Alternatively, cannabinoid derivative compounds using the recombinant host cells and methods of the present disclosure can include one or more additional chemical moieties that are not present in the cannabinoid compound from which it derives. Exemplary chemical moieties that may be added in a cannabinoid derivative include, but are not limited to azido, halo (e.g., chloride, bromide, iodide, fluorine), methyl, alkyl, alkynyl, alkenyl, methoxy, alkoxy, acetyl, amino, carboxyl, carbonyl, oxo, ester, hydroxyl, thio, cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, spirocyclyl, heterospirocyclyl, heterocyclyl, thioalkyl, sulfone, sulfonyl, sulfoxide, amino, alkylamino, dialkylamino, arylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like.

Accordingly, in at least one embodiment, the present disclosure provides a method of producing a derivative compound of a cannabinoid precursor or cannabinoid, wherein the method comprises: (a) culturing in a suitable medium a recombinant host cell of the present disclosure; and (b) recovering the produced derivative compound. In at least one embodiment, the method of producing a derivative compound of a cannabinoid precursor or cannabinoid can further contacting a cell-free extract of the culture of the recombinant host cell containing the produced cannabinoid precursor or cannabinoid with a biocatalytic reagent or chemical reagent capable of converting the cannabinoid precursor or cannabinoid to a derivative compound.

Derivative, compounds of cannabinoid precursor and cannabinoid compounds that can be produced with improved yield using a recombinant host cell of the present disclosure can include derivatives modified (e.g., biocatalytically or synthetically) to provide improved properties of pharmaceutical metabolism and/or pharmacokinetics (e.g. solubility, bioavailability, absorption, distribution, plasma half-life and metabolic clearance). Modifications typically providing such improved pharmaceutical properties can include, but are not limited to, halogenation, acetylation and methylation. It is also contemplated that the derivative compounds of cannabinoids produced by the methods disclosed herein can include pharmaceutically acceptable isotopically labeled compounds. For example, a cannabinoid compound wherein the hydrogen atoms are replaced or substituted by one or more deuterium or tritium atoms. Such isotopically labeled derivatives of cannabinoids can be useful in studies of in vivo pharmacokinetics and tissue distribution.

Upon production by the host cells or in the cell-free mixture of the rare cannabinoid precursors or rare cannabinoid compounds in accordance with the compositions, host cells, and methods of the present disclosure, the desired compounds may be recovered from the host cell suspension or cell-free mixture and separated from other constituents, such as media constituents, cellular debris, etc. Techniques for separation and recovery of the desired compounds are known to those of skill in the art and can include, for example, solvent extraction (e.g. butane, chloroform, ethanol), column chromatography-based techniques, high-performance liquid chromatography (HPLC), for example, and/or countercurrent separation (CCS) based systems. The recovered rare cannabinoid compounds may be obtained in a more or less pure form, for example, the desired rare cannabinoid compound of purity of at least about 60% (w/v), about 70% (w/v), about 80% (w/v), about 90% (w/v), about 95% (w/v) or about 99% (w/v).

It also is contemplated that the cannabinoid, cannabinoid precursor, cannabinoid precursor derivative, or cannabinoid derivative recovered using the methods of the present disclosure can be in the form of a salt. In at least one embodiment, the recovered salt of the cannabinoid, cannabinoid precursor, cannabinoid precursor derivative, or cannabinoid derivative is a pharmaceutically acceptable salt. Such pharmaceutically acceptable salts retain the biological effectiveness and properties of the free base compound.

As described elsewhere herein, the rare cannabinoid compounds provided by the recombinant host cells and methods of the present disclosure are contemplated to have exhibit biological and pharmacological properties like those of the more well-studied cannabinoids such as THC and CBD. Accordingly, in at least one embodiment, the present disclosure also provides a composition comprising a rare cannabinoid, such as a varin cannabinoid, prepared using the recombinant host cells and methods disclosed herein. It is contemplated that the rare cannabinoid compositions provided by the recombinant host cells and methods of the present disclosure can include pharmaceutical compositions, food compositions, and beverage compositions, containing a rare cannabinoid. Generally, compositions comprising rare cannabinoid compounds can further comprise any of the well-known vehicles, excipients and auxiliary substances, such as wetting or emulsifying agents, pH buffering substances and the like, used in the art of formulating pharmaceutical, food, or beverage compositions. For example, pharmaceutical compositions can contain any of the typical pharmaceutically acceptable excipients including, but are not limited to, liquids such as water, saline, polyethylene glycol, hyaluronic acid, glycerol and ethanol. Pharmaceutically acceptable salts can also be included therein, for example, mineral acid salts such as hydrochlorides, phosphates, sulfates, and the like; and the salts of organic acids such as acetates, propionates, benzoates, and the like. In at least one embodiment, a pharmaceutical composition can comprise a pharmaceutically acceptable excipient that serves as a stabilizer of the rare cannabinoid composition. Examples of suitable excipients that also act as stabilizers include, without limitation, pharmaceutical grades of dextrose, sucrose, lactose, sorbitol, inositol, dextran, and the like. Other suitable pharmaceutical excipients can include, without limitation, starch, cellulose, sodium or calcium phosphates, citric acid, glycine, polyethylene glycols (PEGs), and combinations thereof.

### EXAMPLES

Various features and embodiments of the disclosure are illustrated in the following representative examples, which are intended to be illustrative, and not limiting. Those skilled in the art will readily appreciate that the specific examples are only illustrative of the invention as described more fully in the claims which follow thereafter. Every embodiment and feature described in the application should be understood to be interchangeable and combinable with every embodiment contained within.

### Example 1: Biosynthesis of the Rare Cannabinoid, CBGVA from Divarinic Acid, DA, in Saccharomyces cerevisiae Engineered with a Cannabinoid Pathway

This example illustrates a study showing that Saccharomyces cerevisiae CEN.PK2-1 D strains engineered with a pathway capable of converting hexanoic acid (HA) to the cannabinoid, CBGA, are also capable of producing the rare cannabinoid, CBGVA from the precursor compound, divarinic acid (DA). The engineered strains convert HA to CBGA via a pathway comprising genes encoding the enzymes C. sativa AAE1 (SEQ ID NO: 2), OLS (SEQ ID NO: 4), OAC (SEQ ID NO: 6), and PT4 (SEQ ID NO: 10). When cultured in the presence of HA the strain produces the cannabinoid precursor, olivetolic acid (OA) which is then prenylated by the PT4 enzyme to provide the CBGA. The present example illustrates the ability of this same pathway, and particularly the PT4 enzyme, to convert DA to CBGVA.

Materials and Methods

Three yeast strains MV023, MV109, and MV129, which are derived from Saccharomyces cerevisiae strain CEN.PK 2-1 D and include a pathway comprising the enzymes, C. sativa AAE1 (SEQ ID NO: 2), OLS (SEQ ID NO: 4), OAC (SEQ ID NO: 6), and PT4 (SEQ ID NO: 10), were previously shown to produce the cannabinoid CBGA when fed the precursor, olivetolic acid (OA). Each of the MV023, MV109, and MV129 strains were separately grown as 5 mL YPD seed cultures for 18 h at 30° C. on a roller drum. 300 μL cultures then were set up in a plate, inoculated to 0.4 OD, and grown in an incubated shaker at 250 rpm at 30° C. These cultures were fed twice with 1 mM DA (Toronto Research Chemicals, catalog no. D494463) or 1 mM EtOH (control) at 24 h and 48 h and then grown for a further 24 h. Following the 72 h growth, samples were extracted from each 300 μL culture using acetonitrile (ACN), and diluted further for CBGVA detection quantification using an LC/MS at 1:100 dilution.

Samples were analyzed for DA levels using a Thermo Scientific TSQ Fortis LC/MS according to the following procedures and instrumental parameters. The retention time of has to match that of DA authentic standard+/−0.1 min, the mass to charge (m/z) transition values have to be the same of those determined using a DA standard and the ratio of these two m/z transitions has to match that determined using the DA standard+/−20%. DA was quantified using a calibration curve prepared daily using a certified authentic, high purity (>99%) DA standard.

Results: As shown by the results summarized in Table 5 (below), the three yeast strains engineered with a pathway comprising the enzyme PT4 (SEQ ID NO: 10) capable of converting olivetolic acid (OA) to the cannabinoid, CBGA, were also capable of converting the varin cannabinoid precursor, divarinic acid (DA) to the varin cannabinoid, CBGVA.

TABLE 5

| | | CBGVA |
|---|---|---|
| Strain | Feedstock | (mg/L) |
| MV023 | 1mM DA | 82 |
| | 1 mM EtOH (control) | 0.0 |
| MV109 | 1mM DA | 179 |
| | 1 mM EtOH (control) | 0.4 |
| MV129 | 1mM DA | 265 |
| | 1 mM EtOH (control) | 0.0 |

### Example 2: Production of Divarinic Acid (DA) from Butyric Acid (BA) Feedstock in Saccharomyces cerevisiae Transformed with AAE Enzymes not from C. sativa

Example 1 illustrates the ability of yeast engineered with a cannabinoid pathway to convert DA as feedstock to the varin cannabinoid, CBGVA. This example illustrates a study of engineered yeast strains further transformed with a range of 40 different AAE enzymes from source organisms other than C. sativa for the ability to convert a butyric acid (BA) feedstock to the varin cannabinoid precursor compound, divarinic acid, DA. The amino acid sequences of the 40 candidate AAE enzymes each have less than 6% sequence similarity to the AAE1 polypeptide of SEQ ID NO: 2. Briefly, heterologous nucleic acids encoding the 40 candidate AAE enzymes were homologously transformed into the CEN.PK2-1 D strain of Saccharomyces cerevisiae, which previously has been engineered with a pathway of the enzymes AAE1 (SEQ ID NO: 2), OLS (SEQ ID NO: 4), OAC (SEQ ID NO: 6), and is capable of converting hexanoic acid (HA) feedstock to the cannabinoid precursor, olivetolic acid (OA). The homologous transformation vector was designed to integrate the candidate AAE (in place of AAE1). The transformants were screened for the ability to produce DA when cultured in BA feedstock.

Materials and Methods

A. Transformation of Yeast Strains Expressing Candidate AAE Genes

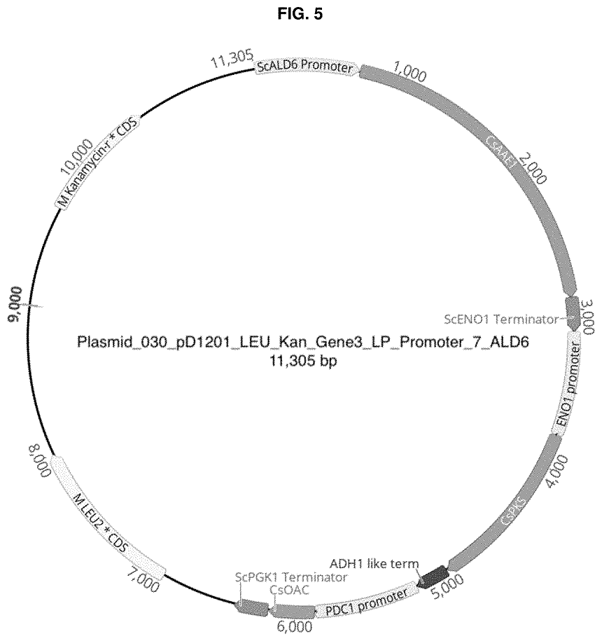

Nucleotide and encoded amino acid sequences of the 40 candidate AAE genes derived from organisms are provided as SEQ ID NOs: 15-98 in Table 3 and the accompanying Sequence Listing. The amino acid sequences encoded by the 40 candidate AAE genes were compared to AAE1 from C. sativa (SEQ ID NO: 2) and found to have 5% or lower amino acid sequence identity. The 40 candidate AAE genes were yeast-codon-optimized, synthesized, and the synthesized genes were used to co-transform the yeast strain CEN.PK2-1 D with the linearized plasmid_030 minus CsAAE1 depicted in FIG. 5 for homologous recombination. The transformed strains were tested for the presence of the recombined AAE candidate genes using PCR. The AAE candidate genes were all ˜500 bp shorter than CsAAE1, accordingly the amplicon length was used to determine transformation using PCR with the following primers:

FB_BB_AAE_Homologs:
(SEQ ID NO: 99)
5′-AACATCTTTAACATACACAAACACATACTATCAGAATACAATGGGA
AAAAATTATAAGTC-3′.
RP_AAE_Homolog_BB:
(SEQ ID NO: 100)
5′-AAAAACGTGTTTTTTGGACTAGAAGGCTTAATCAAAAGCTTTACTC
AAAATGACTAAACT-3′

B. Screening Transformants for BA to DA Conversion

Colonies for individual transformed strains were used to inoculate 300 μL of Sc-Leu in 96-well plates. After 24 h wells were diluted 1:10. The wells were fed 1 mM BA 24 h and 48 h after this dilution and extracted at 72 h using acetonitrile (ACN) at a 1:1 culture volume to ACN ratio. The plates were grown in a 30 degree C. incubator at 900 rpm and 89% humidity. Samples were analyzed for DA levels using a Thermo Scientific TSQ Fortis LC/MS as described in Example 1.

Results: A total of 11 of the 40 candidate AAE enzymes were identified as capable of producing DA from BA feedstock. Table 6 (below) summarizes the amount of DA produced by the 11 AAE transformant strains that were found to produce the rare cannabinoid precursor from the BA feedstock.

TABLE 6

| | | DA | DA | SEQ ID | SEQ ID |
|---|---|---|---|---|---|
| AAE | Source Organism | production | Relative | NO: | NO: |
| Abbrev. | (AA Sequence) | (mg/mL) | production | (nt) | (aa) |
| CsAAE1 | Cannabis sativa | 0.68 | 1 | 1 | 2 |
| | (SEQ ID NO: 2) | | | | |
| TM4 | Taxus x media | 1.18 | 1.74 | 15 | 16 |
| | (SEQ ID NO: 16) | | | | |
| CCL2 | Humulus lupulus | 0.98 | 1.44 | 17 | 18 |
| | (SEQ ID NO: 18) | | | | |
| CM1 | Callitris macleayana | 0.96 | 1.41 | 19 | 20 |
| | (SEQ ID NO: 20) | | | | |
| DA1 | Diselma archeri | 0.82 | 1.21 | 21 | 22 |
| | (SEQ ID NO: 22) | | | | |
| CCL3 | Humulus lupulus | 0.71 | 1.04 | 23 | 24 |
| | (SEQ ID NO: 24) | | | | |
| AA1 | Amentotaxus | 0.57 | 0.84 | 25 | 26 |
| | argotaenia | | | | |
| | (SEQ ID NO: 26) | | | | |
| WC1 | Widdringtonia | 0.46 | 0.68 | 27 | 28 |
| | cedarbergensis | | | | |
| | (SEQ ID NO: 28) | | | | |
| CH3 | Cephalotaxus | 0.35 | 0.51 | 29 | 30 |
| | harringtonia | | | | |
| | (SEQ ID NO: 30) | | | | |
| CH2 | Cephalotaxus | 0.30 | 0.44 | 31 | 32 |
| | harringtonia | | | | |
| | (SEQ ID NO: 32) | | | | |
| PA1 | Prumnopitys andina | 0.26 | 0.38 | 33 | 34 |
| | (SEQ ID NO: 34) | | | | |
| TM5 | Taxus x media | 0.17 | 0.25 | 35 | 36 |
| | (SEQ ID NO: 36) | | | | |

At least five of the AAE transformants were observed to produce DA from BA feeding in amount greater than that produced by the strain engineered with the AAE1 enzyme from C. sativa. These increased DA production of the five AAE enzymes of SEQ ID NO: 16 (TM4), 18 (CCL2), 20 (CM1), 22 (DA1), and 24 (CCL3) suggests that they may be particularly useful as heterologous AAE enzymes for biosynthesis of the cannabinoid precursor, DA, in yeast and other recombinant host cells.

### Example 3: Production of CBGVA from Butyric Acid (BA) Feedstock in Saccharomyces cerevisiae Transformed with AAE Enzymes not from C. sativa

This example illustrates the ability of Saccharomyces cerevisiae CEN.PK2-1 D strains engineered with a cannabinoid pathway comprising an AAE enzyme not from C. sativa of Example 2 to convert butyric acid (BA) as feedstock to the varin cannabinoid, CBGVA.

Materials and Methods

Genes encoding the following AAE candidate enzymes TM4 (SEQ ID NO: 16), CCL2 (SEQ ID NO: 18), CM1 (SEQ ID NO: 20), DA1 (SEQ ID NO: 22), CCL3 (SEQ ID NO: 24), AA1 (SEQ ID NO: 26), WC1 (SEQ ID NO: 28), CH3 (SEQ ID NO: 30), CH2 (SEQ ID NO: 32), PA1 (SEQ ID NO: 34), and TM5 (SEQ ID NO: 36), were synthesized by TWIST Biosciences and assembled with 5′ and 3′ homology arms (DONOR DNA) using Overlap Extension polymerase chain reaction (OE-PCR). DONOR DNA and gRNA cassette were then transformed into a Saccharomyces cerevisiae CEN.PK2-1 D strain, MV034, which was already engineered with the genes encoding the cannabinoid pathway enzymes OLS (SEQ ID NO: 4), OAC (SEQ ID NO: 6), and PT4 (SEQ ID NO: 10) integrated into the proper loci via homologous recombination. Proper AAE gene integration was characterized by direct colony PCR using promoter and terminator sequences as template for oligo design and two additional internal primers (along the candidate AAE). Colonies for individual transformed strains were used to inoculate 300 μL of Sc-Leu in 96-well plates. After 24 h wells were diluted 1:10. Assays measuring in vivo production of DA and CBGVA were carried out by feeding 1 mM BA at 4, 24 and 48 hours after inoculation with samples harvested after 72 hours. Sample extracts were analyzed by LC-MS and chromatogram peaks were compared to commercial standards as described in Examples 1 and 2. The theoretical m/z values of rare cannabinoid precursor, DA and the rare cannabinoid, CBGVA were selected from each chromatogram (CBGVA=287.2/313.2; DA=109.1/151.0). Data are mean+/−s.d.; n=4 independent samples.

Results

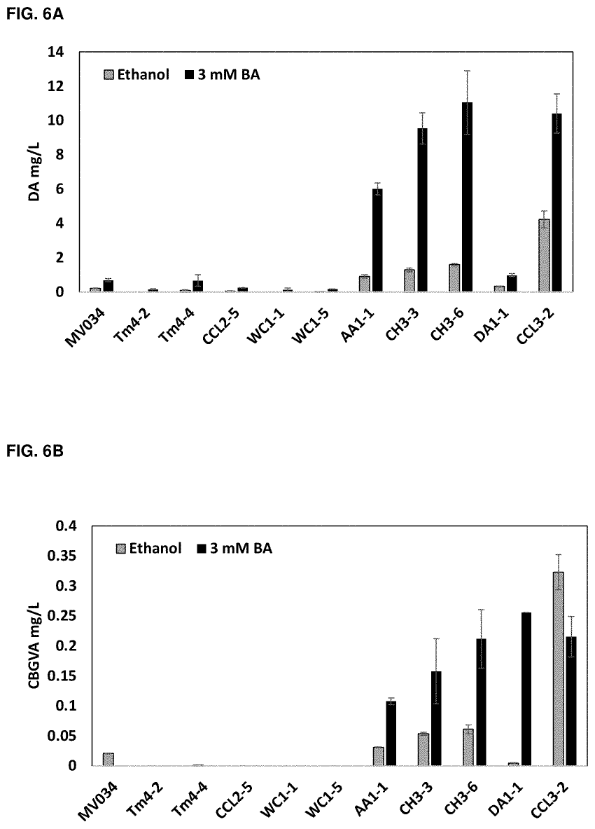

As shown by the plot depicted in FIG. 6A, the strains engineered with an integrated cannabinoid pathway including one of the candidate AAE enzymes AA1 (SEQ ID NO: 26), CH3 (SEQ ID NO: 30), or CCL3 (SEQ ID NO: 24), exhibited greatly increased production of the rare cannabinoid precursor compound, DA (between about 6 mg/L and 12 mg/L) when fed BA, relative to the production of DA by a control yeast strain (MV034) that includes the AAE enzyme from C. sativa, AAE1 (SEQ ID NO: 2). Additionally, it was observed that the AAE enzyme from Cephalotaxus harringtonia, CH3, exhibited significantly increased DA production in two different transformed yeast strains, denoted “CH3-3” and “CH3-6,” where the CH3 gene was integrated at different loci.

As shown by the plot depicted in FIG. 6B, the same yeast strains including one of the candidate AAE enzymes AA1 (SEQ ID NO: 26), CH3 (SEQ ID NO: 30), or CCL3 (SEQ ID NO: 24), also exhibited greatly increased production of the rare cannabinoid, CBGVA (between about 0.1 mg/L and 0.25 mg/L) when fed BA, relative to the production of the control strain, MV034, containing the AAE1 enzyme from C. sativa. Additionally, the AAE candidate, DA1 (SEQ ID NO: 22) also exhibited greatly enhanced CBGVA production when fed BA, although as shown by the results depicted in FIG. 6A, it did not exhibit the high levels of the precursor, DA production, that was exhibited by the strains with the AAE enzymes, AA1, CH3, or CCL3.

### Example 4: Production of CBGA from Hexanoic Acid (HA) Feedstock in Saccharomyces cerevisiae Transformed with CCL3 Enzyme from Humulus lupulus

This example illustrates the ability of a Saccharomyces cerevisiae CEN.PK2-1 D strain engineered with a cannabinoid pathway comprising the AAE enzyme from Humulus lupulus, CCL3 (SEQ ID NO: 24) to convert hexanoic acid (HA) as feedstock to the cannabinoid, CBGA.

Materials and Methods

Strain Build: The gene encoding the AAE enzyme from Humulus lupulus, CCL3 (SEQ ID NO: 24) was amplified from Humulus lupulus cDNA and integrated into a parent Saccharomyces cerevisiae strain that had already been engineered with genes encoding a cannabinoid pathway enzymes OLS (SEQ ID NO: 4), OAC (SEQ ID NO: 6), and PT4 (SEQ ID NO: 10) as described in Example 3. The CCL3 gene was integrated into the XI-2, X4, and POX1 loci via homologous recombination generating a new strain named “MV483.” Proper integration of the CCL3 gene in strain MV483 was characterized by direct colony PCR using promoter and terminator sequences as template for oligo design and two additional internal primers (along the candidate).

Next, the pGal1 promoter driving the expression of the three CCL3 copies in MV483 was replaced with pALD6 (the promoter for the Saccharomyces cerevisiae gene ALD6) to modify its expression profile. The promoter was amplified from CEN.PK2-1 D genomic DNA and integrated upstream and adjacent to the three CCL3 copies using homologous recombination to generate a new strain named “MV499.” Proper integration of pALD6 was characterized by direct colony PCR using promoter and terminator sequences as template for oligo design and two additional internal primers (along the candidate).

B. Screening of Clones for CBGA Biosynthesis:

Individual clones from the MV499 strain and the MV483 parent strain were picked and grown in 0.3 mL YPD in 96-well plates. The culture plates were incubated in shaking incubators for 24 h at 30 C, 90% humidity, and 600 rpm (3 mm throw). Cultures were then sub-cultured into 0.27 mL fresh YPD and fed with hexanoic acid (HA) to 3 mM final concentration. Subculture plates were grown in shaking incubators for 72 hours at 30 C, 90% humidity, and 600 rpm (3 mm throw). The whole broth from these sub-culture plates was extracted and analyzed for the presence of the cannabinoid CBGA, using HPLC, as described below.

1. LC-MS/MS sample preparation: Whole culture broth was extracted in 100% methanol and diluted with 100% methanol for sample preparation. The prepared samples were loaded onto UHPLC coupled to a triple quadrupole mass spectrometry detector. The metabolites OA and CBGA were detected using SRM mode. Calibration curves of OA and CBGA were generated by running serial dilutions of standards, and then used to calculate concentrations of each metabolite.

2. UHPLC MS instrumentation and parameters: UHPLC system: A Thermo Scientific Vanquish™ UHPLC Systems equipped with a pump (VF-P10-A), an autosampler (VF-A10-A), and a column compartment (VH-C10-A) was used for the chromatographic separation. Separation was achieved with a Thermo Accucore™ C18 column, 2.6 μm, 150×2.1 mm (Thermo Scientific) at 40° C., with an injection volume 2 μL. The mobile phase consists of 0.1% formic acid in water (A) and 0.1% formic acid in acetonitrile (B). The flow rate is 0.8 mL/min, and the gradient elution program is as follows: 10-95% B (0-1.0 min), 95% B (1.0-2.5 min), 95-10% B (2.5-2.6 min), and 10% B (2.6-3.5 min). Seal wash 10% acetonitrile in water. Needle wash IPA:water:methanol:acetonitrile (1:1:1:1).

Mass spectrometry measurements were performed on an Thermo Scientific TSQ Altis™ triple quadrupole mass. Samples were introduced to MS via electrospray ionization (ESI) in negative mode with selected reaction monitoring (SRM). Mass spectrometer was operated in the following conditions: sheath gas flow rate, 60 Arb; auxiliary gas, 15 Arb. The ESI voltage 2900 V and the source temperature was 350° C. The parameter of the quantification of SRM transitions for CBGA are shown below in Table 7.

TABLE 7
Parameters for quantification SRM transitions for CBGA.

| | Retention | RT Window | Precursor | Product | Collision |
|---|---|---|---|---|---|
| Compound | Time (min) | (min) | (m/z) | (m/z) | Energy (V) |
| CBGA | 0.8 | 0.5 | 359.19 | 315.29 | 21 |
| CBGA | 0.8 | 0.5 | 359.19 | 341.15 | 19 |

Results

As shown by the results shown in Table 8, the MV499 strain with pALD6 driving the expression of the three CCL3 copies was capable of producing CBGA with a titer approximately 3-fold greater than the CBGA titer produced by the MV483 parent strain in which pGal1 drives the expression of CCL3.

TABLE 8
CBGA titers produced by
strains MV483 and MV499

| Strain | CBGA titer (mg/L) |
|---|---|
| MV483 | 18.0 ± 3.5 |
| MV499 | 55.9 ± 0.9 |

### Example 5: Production of OA from Hexanoic Acid (HA) Feedstock in Saccharomyces cerevisiae Transformed with CCL3 Enzyme from Humulus lupulus

This example illustrates the ability of a Saccharomyces cerevisiae CEN.PK2-1 D strain engineered with a cannabinoid pathway comprising the AAE enzyme from Humulus lupulus, CCL3 (SEQ ID NO: 24) to convert hexanoic acid (HA) as feedstock to the cannabinoid precursor, OA.

Materials and Methods

Strain Build: The gene encoding the AAE enzyme from Humulus lupulus, CCL3 (SEQ ID NO: 24) was amplified from Humulus lupulus cDNA and integrated into a Saccharomyces cerevisiae CEN.PK2-1 D strain, as a three-gene cassette along with genes encoding the cannabinoid pathway enzymes OLS (SEQ ID NO: 4) and OAC (SEQ ID NO: 6). The CCL3-OLS-OAC cassette was integrated into the X4 locus via homologous recombination and the strain named “MV505”. Proper integration of the CCL3-OLS-OAC cassette was characterized by direct colony PCR using promoter and terminator sequences as template for oligo design and two additional internal primers for each of the three genes. A strain “MV002-pALD6” comprising a single three-gene cassette with C. sativa AAE1 (SEQ ID NO: 2) under the ALD6 promoter, as well as, OLS (SEQ ID NO: 4) and OAC (SEQ ID NO: 6), was used as a control in screening.

B. Screening for OA biosynthesis:

Individual clones from the MV505 strain, the MV000P parent strain, and MV002-pALD6 were picked and grown in 0.3 mL YPD in 96-well plates. The culture plates were incubated in shaking incubators for 24 h at 30 C, 90% humidity, and 600 rpm (3 mm throw). Cultures were then sub-cultured into 0.27 mL fresh YPD and fed with hexanoic acid (HA) to 3 mM final concentration. Subculture plates were grown in shaking incubators for 72 hours at 30 C, 90% humidity, and 600 rpm (3 mm throw). The whole broth from these sub-culture plates was extracted and analyzed for the presence of the cannabinoid precursor compound, OA, using HPLC, as described below. This was repeated two more times for a total of three separate experiments.

1. LC-MS/MS sample preparation: The whole broth of the culture was extracted in 100% methanol and diluted with 100% methanol for sample preparation. The prepared samples were loaded onto UHPLC coupled to a triple quadrupole mass spectrometry detector. Metabolites OA and CBGA were detected using SRM mode. Calibration curves of OA and CBGA were generated by running serial dilutions of standards, and then used to calculate concentrations of each metabolite.

2. UHPLC MS instrumentation and parameters: UHPLC system: A Thermo Scientific Vanquish™ UHPLC Systems equipped with a pump (VF-P10-A), an autosampler (VF-A10-A), and a column compartment (VH-C10-A) was used for the chromatographic separation. Separation was achieved with a Thermo Accucore™ C18 column, 2.6 μm, 150×2.1 mm (Thermo Scientific) at 40° C., with an injection volume 2 μL. The mobile phase consists of 0.1% formic acid in water (A) and 0.1% formic acid in acetonitrile (B). The flow rate is 0.8 mL/min, and the gradient elution program is as follows: 10-95% B (0-1.0 min), 95% B (1.0-2.5 min), 95-10% B (2.5-2.6 min), and 10% B (2.6-3.5 min). Seal wash 10% acetonitrile in water. Needle wash IPA:water:methanol:acetonitrile (1:1:1:1).

Mass spectrometry measurements were performed on an Thermo Scientific TSQ Altis™ triple quadrupole mass. Samples were introduced to MS via electrospray ionization (ESI) in negative mode with selected reaction monitoring (SRM). Mass spectrometer was operated in the following conditions: sheath gas flow rate, 60 Arb; auxiliary gas, 15 Arb. The ESI voltage 2900 V and the source temperature was 350° C. The parameter of the quantification of SRM transitions for OA are shown below in Table 9.

TABLE 9
Parameters for quantification of SRM transitions for OA.

| | Retention | RT Window | Precursor | Product | Collision |
|---|---|---|---|---|---|
| Compound | Time (min) | (min) | (m/z) | (m/z) | Energy (V) |
| OA | 0.5 | 0.5 | 223 | 137.1 | 21 |
| OA | 0.5 | 0.5 | 223 | 179.1 | 15 |

As shown by the data shown in Table 10, strain MV505, with the three-gene cassette comprising of CCL3-OLS-OAC at the X4 locus, produced an OA titer comparable to the OA titer produced by MV002-pALD6 during three separate HTP assay experiments.

TABLE 10
OA titers produced by strains MV002-pALD6 and MV505

| | Experiment | | |
|---|---|---|---|
| Strain | #1 | #2 | #3 |
| MV002-pALD6 | 133.5 ± 8.1 mg/L OA | 126.6 ± 12.7 mg/L OA | 138.6 ± 3.5 mg/L OA |
| MV505 | 77.0 ± 14.2 mg/L OA | 78.1 ± 15.4 mg/L OA | 105.3 ± 25.6 mg/L OA |

While the foregoing disclosure of the present invention has been described in some detail by way of example and illustration for purposes of clarity and understanding, this disclosure including the examples, descriptions, and embodiments described herein are for illustrative purposes, are intended to be exemplary, and should not be construed as limiting the present disclosure. It will be clear to one skilled in the art that various modifications or changes to the examples, descriptions, and embodiments described herein can be made and are to be included within the spirit and purview of this disclosure and the appended claims. Further, one of skill in the art will recognize a number of equivalent methods and procedure to those described herein. All such equivalents are to be understood to be within the scope of the present disclosure and are covered by the appended claims.

Additional embodiments of the invention are set forth in the following claims.

The disclosures of all publications, patent applications, patents, or other documents mentioned herein are expressly incorporated by reference in their entirety for all purposes to the same extent as if each such individual publication, patent, patent application or other document were individually specifically indicated to be incorporated by reference herein in its entirety for all purposes and were set forth in its entirety herein. In case of conflict, the present specification, including specified terms, will control.

1. A recombinant host cell which produces a cannabinoid precursor and/or a cannabinoid, wherein the cell comprises a pathway of enzymes AAE, OLS, OAC, and optionally, PT4, wherein the AAE has an amino acid sequence of at least 70% identity to a sequence selected from: CCL3 (SEQ ID NO: 24), CH3 (SEQ ID NO: 30), TM4 (SEQ ID NO: 16), CCL2 (SEQ ID NO: 18), CM1 (SEQ ID NO: 20), DA1 (SEQ ID NO: 22), AA1 (SEQ ID NO: 26), WC1 (SEQ ID NO: 28), CH2 (SEQ ID NO: 32), PA1 (SEQ ID NO: 34), and TM5 (SEQ ID NO: 36).

2. The cell of claim 1, wherein:
(a) the pathway catalyzes the reactions (i)(a)-(iii)(a) and/or (i)(b)-(iii)(b): 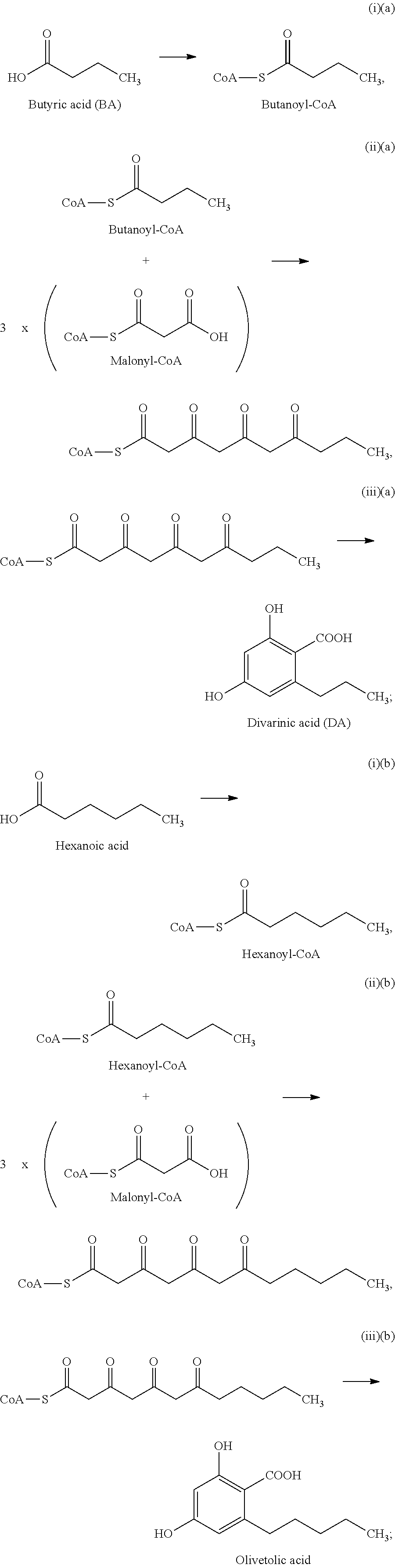
and/or
(b) the pathway enzymes OLS, and OAC have an amino acid sequence of at least 90% identity to SEQ ID NO: 4 (OLS), and SEQ ID NO: 6 (OAC), respectively.

3. The cell of claim 1, wherein:
(a) the pathway catalyzes reaction (iv)(a) and/or (iv)(b):
(iv)(a) 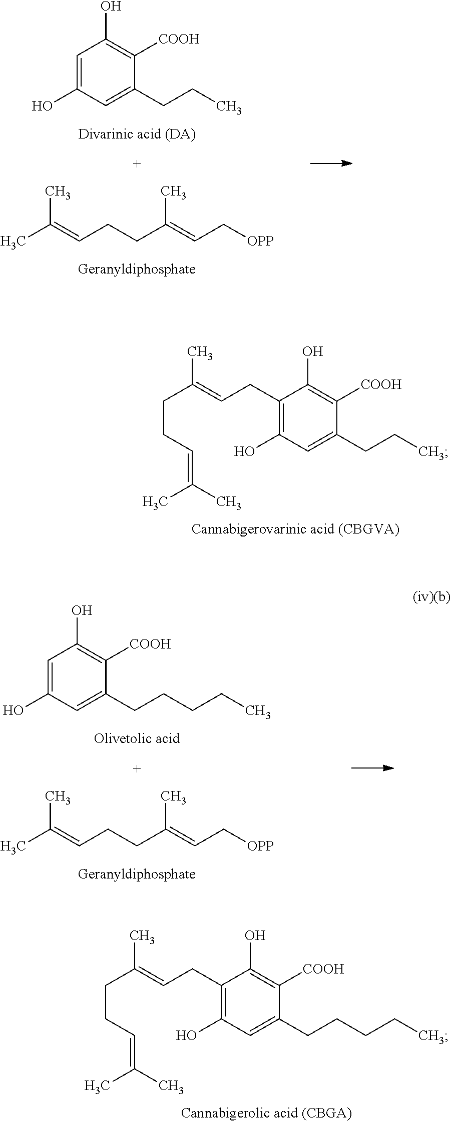
and/or
(b) the pathway comprises the enzyme PT4; optionally, wherein the PT4 has an amino acid sequence of at least 90% identity to SEQ ID NO: 8 or 10 (PT4) respectively.

4. The cell of claim 1, wherein:
(a) the recombinant host cell pathway further comprises an enzyme capable of catalyzing a reaction (v)(a), (vi)(a), (vii)(a), (v)(b), (vi)(b), and/or (vii)(b): 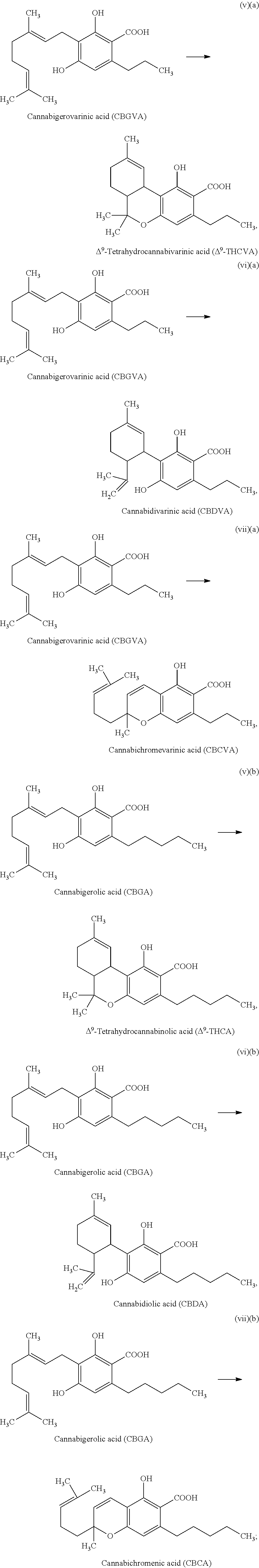
and/or
(b) the pathway comprises an enzyme THCA synthase, CBDA synthase, and/or CBCA synthase; optionally, the enzyme CBDA synthase having an amino acid sequence of at least 90% identity to SEQ ID NO: 12 or 14, and/or the enzyme THCA synthase having at least 90% identity to SEQ ID NO: 102 or 104.

5. The cell of claim 1, wherein:
(a) the cell produces divarinic acid (DA) and/or cannabigerovarinic acid (CBGVA) when cultured in the presence of butyric acid (BA);
(b) the cell produces olivetolic acid (OA) and/or cannabigerolic acid (CBGA) when cultured in the presence of hexanoic acid (HA);
(c) the amount of DA and/or CBGVA the cell produces when cultured in the presence of BA is increased relative to the amount of DA and/or CBGVA produced by a control cell comprising a pathway of enzymes AAE, OLS, and OAC, wherein the control cell AAE is AAE1 from C. sativa comprising the amino acid sequence of SEQ ID NO: 2;
(d) the amount of OA and/or CBGA the cell produces when cultured in the presence of HA is increased relative to the amount of OA and/or CBGA produced by a control cell comprising a pathway of enzymes AAE, OLS, and OAC, wherein the control cell AAE is AAE1 from C. sativa comprising the amino acid sequence of SEQ ID NO: 2; and/or
(e) the amount of DA, CBGVA, OA, and/or CBGA produced by the cell is increased by at least 1.1-fold, at least 1.2-fold, at least 1.5-fold, at least 2-fold, at least 3-fold, at least 5-fold, at least 10-fold, or more relative to the control cell comprising a pathway of enzymes AAE, OLS, and OAC, wherein the control cell AAE is AAE1 from C. sativa comprising the amino acid sequence of SEQ ID NO: 2.

6. The cell of claim 1, wherein the cell produces a cannabinoid selected from cannabigerolic acid (CBGA), cannabigerol (CBG), cannabidiolic acid (CBDA), cannabidiol (CBD), Δ9-tetrahydrocannabinolic acid (Δ9-THCA), Δ9-tetrahydrocannabinol (Δ9-THC), Δ8-tetrahydrocannabinolic acid (Δ8-THCA), Δ8-tetrahydrocannabinol (Δ8-THC), cannabichromenic acid (CBCA), cannabichromene (CBC), cannabinolic acid (CBNA), cannabinol (CBN), cannabidivarinic acid (CBDVA), cannabidivarin (CBDV), Δ9-tetrahydrocannabivarinic acid (Δ9-THCVA), Δ9-tetrahydrocannabivarin (Δ9-THCV), cannabidibutolic acid (CBDBA), cannabidibutol (CBDB), Δ9-tetrahydrocannabutolic acid (Δ9-THCBA), Δ9-tetrahydrocannabutol (Δ9-THCB), cannabidiphorolic acid (CBDPA), cannabidiphorol (CBDP), Δ9-tetrahydrocannabiphorolic acid (Δ9-THCPA), Δ9-tetrahydrocannabiphorol (Δ9-THCP), cannabichromevarinic acid (CBCVA), cannabichromevarin (CBCV), cannabigerovarinic acid (CBGVA), cannabigerovarin (CBGV), cannabicyclolic acid (CBLA), cannabicyclol (CBL), cannabielsoinic acid (CBEA), cannabielsoin (CBE), cannabicitranic acid (CBTA), cannabicitran (CBT), and any combination thereof.

7. The cell of claim 1, wherein the source organism of the recombinant host cell is selected from Saccharomyces cerevisiae, Yarrowia Pichia pastoris, and Escherichia coli.

8. The cell of claim 1, wherein the recombinant host cell is Saccharomyces cerevisiae and the gene encoding the AAE enzyme is under the control of an ALD6 promoter.

9. A method for producing a cannabinoid precursor and/or a cannabinoid comprising:
(a) culturing a recombinant host cell of claim 1 in a suitable medium comprising butyric acid (BA) and/or hexanoic acid (HA); and
(b) recovering the produced divarinic acid (DA), cannabigerovarinic acid (CBGVA), olivetolic acid (OA), and/or cannabigerolic acid (CBGA).

10. A method for producing a cannabinoid precursor and/or a cannabinoid comprising:
(a) culturing in a suitable medium comprising butyric acid (BA) and/or hexanoic acid (HA), a recombinant host cell comprising a pathway of enzymes AAE, OLS, and OAC, wherein the AAE has an amino acid sequence of at least 70% identity to a sequence selected from: CCL3 (SEQ ID NO: 24), CH3 (SEQ ID NO: 30), TM4 (SEQ ID NO: 16), CCL2 (SEQ ID NO: 18), CM1 (SEQ ID NO: 20), DA1 (SEQ ID NO: 22), AA1 (SEQ ID NO: 26), WC1 (SEQ ID NO: 28), CH2 (SEQ ID NO: 32), PA1 (SEQ ID NO: 34), and TM5 (SEQ ID NO: 36); and
(b) recovering the produced cannabinoid precursor and/or a cannabinoid.

11. The method of claim 10, wherein:
(a) the pathway catalyzes the reactions (i)(a)-(iii)(a) and/or (i)(b)-(iii)(b): 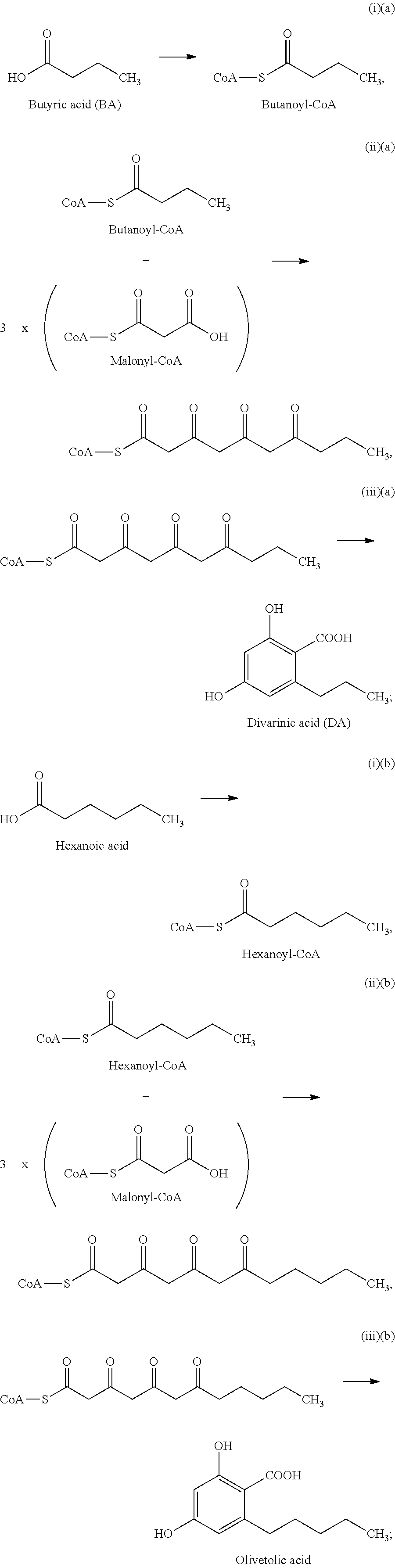
and/or
(b) the pathway enzymes OLS, and OAC have an amino acid sequence of at least 90% identity to SEQ ID NO: 4 (OLS), and SEQ ID NO: 6 (OAC), respectively.

12. The method of claim 10, wherein:
(a) the pathway catalyzes reaction (iv)(a) and/or (iv)(b):
(iv)(a) 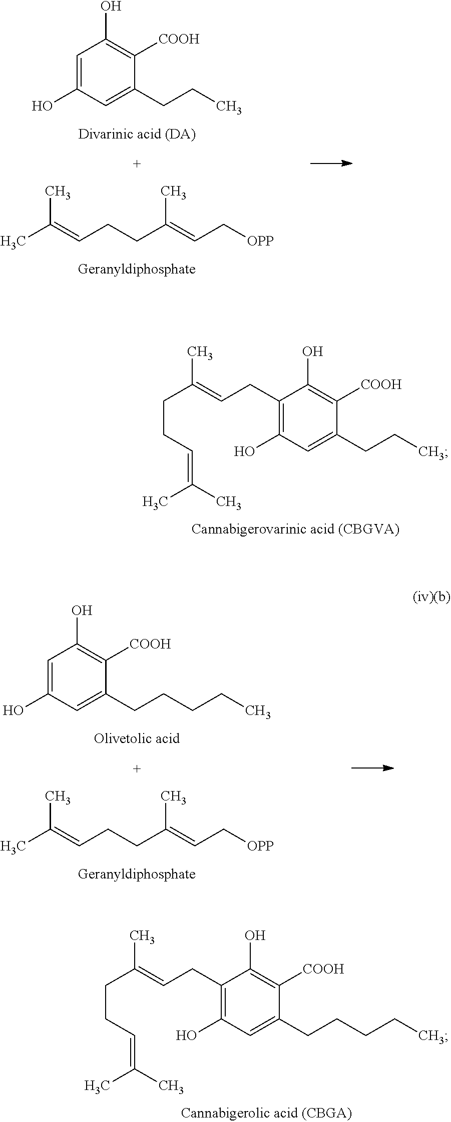
and/or
(b) the pathway comprises the enzyme PT4; optionally, wherein the PT4 has an amino acid sequence of at least 90% identity to SEQ ID NO: 8 or 10 (PT4) respectively.

13. The method of claim 10, wherein:
(a) the recombinant host cell pathway further comprises an enzyme capable of catalyzing a reaction (v)(a), (vi)(a), (vii)(a), (v)(b), (vi)(b), and/or (vii)(b): 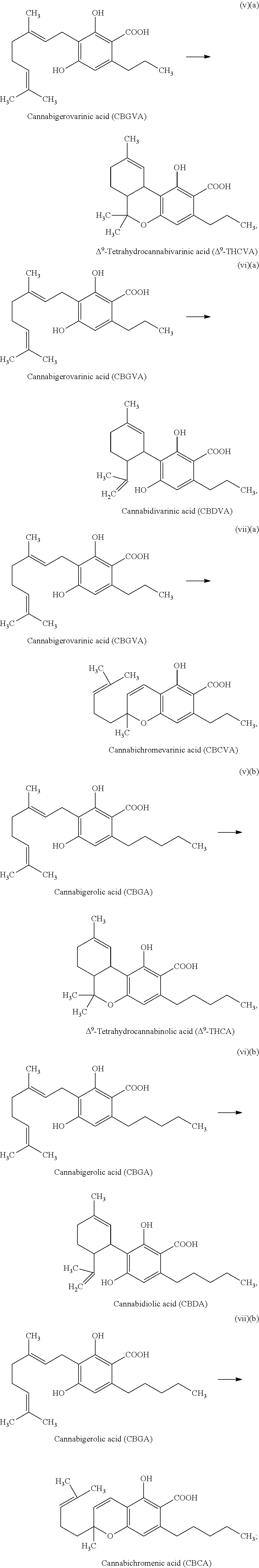
and/or
(b) the pathway comprises an enzyme THCA synthase, CBDA synthase, and/or CBCA synthase; optionally, the enzyme CBDA synthase having an amino acid sequence of at least 90% identity to SEQ ID NO: 12 or 14, and/or the enzyme THCA synthase having at least 90% identity to SEQ ID NO: 102 or 104.

14. The method of claim 10, wherein:
(a) the cell produces divarinic acid (DA) and/or cannabigerovarinic acid (CBGVA) when cultured in the presence of butyric acid (BA);
(b) the cell produces olivetolic acid (OA) and/or cannabigerolic acid (CBGA) when cultured in the presence of hexanoic acid (HA);
(c) the amount of DA and/or CBGVA the cell produces when cultured in the presence of BA is increased relative to the amount of DA and/or CBGVA produced by a control cell comprising a pathway of enzymes AAE, OLS, and OAC, wherein the control cell AAE is AAE1 from C. sativa comprising the amino acid sequence of SEQ ID NO: 2; and/or
(d) the amount of OA and/or CBGA the cell produces when cultured in the presence of HA is increased relative to the amount of OA and/or CBGA produced by a control cell comprising a pathway of enzymes AAE, OLS, and OAC, wherein the control cell AAE is AAE1 from C. sativa comprising the amino acid sequence of SEQ ID NO: 2;
(e) the amount of DA, CBGVA, OA, and/or CBGA produced by the cell is increased by at least 1.1-fold, at least 1.2-fold, at least 1.5-fold, at least 2-fold, at least 3-fold, at least 5-fold, at least 10-fold, or more relative to the control cell comprising a pathway of enzymes AAE, OLS, and OAC, wherein the control cell AAE is AAE1 from C. sativa comprising the amino acid sequence of SEQ ID NO: 2.

15. The method of claim 10, wherein source organism of the recombinant host cell is selected from Saccharomyces cerevisiae, Yarrowia lipolytica, Pichia pastoris, and Escherichia coli.

16. The cell of claim 10, wherein the recombinant host cell is Saccharomyces cerevisiae and the gene encoding the AAE enzyme is under the control of an ALD6 promoter.

17. The method of claim 10, wherein the method further comprises contacting a cell-free extract of the culture with a biocatalytic reagent or chemical reagent.

18. A method for producing a varin cannabinoid comprising:
(a) culturing in a suitable medium comprising butyric acid (BA), a recombinant host cell comprising a pathway of enzymes AAE, OLS, and OAC, wherein the AAE has an amino acid sequence of at least 70% identity to a sequence selected from: CCL3 (SEQ ID NO: 24), CH3 (SEQ ID NO: 30), TM4 (SEQ ID NO: 16), CCL2 (SEQ ID NO: 18), CM1 (SEQ ID NO: 20), DA1 (SEQ ID NO: 22), AA1 (SEQ ID NO: 26), WC1 (SEQ ID NO: 28), CH2 (SEQ ID NO: 32), PA1 (SEQ ID NO: 34), and TM5 (SEQ ID NO: 36), and wherein the host cell produces divarinic acid (DA) when cultured in the presence of butyric acid (BA); and
(b) recovering the produced varin cannabinoid.
